SEQUENCE LISTING (84 sequences)

SEQ ID NO: 1 - protein, 36 aa, Artificial Sequence
  Description of Artificial Sequence Synthetic polypeptide
GSSCEPGRTFEDECNTCRCGADGRSAACTLEACPNQ

SEQ ID NO: 2 - protein, 36 aa, Artificial Sequence
  Description of Artificial Sequence Synthetic polypeptide
GSSCEPGRTFADACNTCRCGADGRSAACTLAACPNQ

SEQ ID NO: 3 - protein, 36 aa, Artificial Sequence
  Description of Artificial Sequence Synthetic polypeptide
GSSCEPGKTFRDRCNTCRCGADGRSAACTLRACPNQ

SEQ ID NO: 4 - protein, 36 aa, Artificial Sequence
  Description of Artificial Sequence Synthetic polypeptide
GSSCEPGRTFKDRCNTCRCGADGRSAACTLRACPNQ

SEQ ID NO: 5 - protein, 36 aa, Artificial Sequence
  Description of Artificial Sequence Synthetic polypeptide
GSSCEPGRTFRDKCNTCRCGADGRSAACTLRACPNQ

SEQ ID NO: 6 - protein, 36 aa, Artificial Sequence
  Description of Artificial Sequence Synthetic polypeptide
GSSCEPGRTFRDRCNTCRCGADGKSAACTLRACPNQ

SEQ ID NO: 7 - protein, 36 aa, Artificial Sequence
  Description of Artificial Sequence Synthetic polypeptide
GSSCEPGRTFRDRCNTCRCGADGRSAACTLKACPNQ

SEQ ID NO: 8 - protein, 35 aa, Artificial Sequence
  Description of Artificial Sequence Synthetic polypeptide
GSSCEPGTTFRDRCNTCRCGSDGRSAACTLRACPQ

SEQ ID NO: 9 - protein, 38 aa, Artificial Sequence
  Description of Artificial Sequence Synthetic polypeptide
GSSCTPGTTFRDRCNTCRCSSNGRSAACTLRACPPGSY

SEQ ID NO: 10 - protein, 38 aa, Artificial Sequence
  Description of Artificial Sequence Synthetic polypeptide
GSSCTPGTTFRNRCNTCRCGSNGRSASCTLMACPPGSY

SEQ ID NO: 11 - protein, 38 aa, Artificial Sequence
  Description of Artificial Sequence Synthetic polypeptide
GSSCTPGATFRNRCNTCRCGSNGRSASCTLMACPPGSY

SEQ ID NO: 12 - protein, 33 aa, Artificial Sequence
  Description of Artificial Sequence Synthetic polypeptide
GSSCQPGTTYQRGCNTCRCLEDGQTEACTLRLC

SEQ ID NO: 13 - protein, 38 aa, Artificial Sequence
  Description of Artificial Sequence Synthetic polypeptide
GSSCTPGATYREGCNICRCRSDGRSGACTRRICPVDSN

SEQ ID NO: 14 - protein, 34 aa, Artificial Sequence
  Description of Artificial Sequence Synthetic polypeptide
GSSCQPGTTFRRDCNTCVCNRDGTNAACTLRACL

SEQ ID NO: 15 - protein, 36 aa, Artificial Sequence
  Description of Artificial Sequence Synthetic polypeptide
GSSCEPGRTFRDRCNTCRCGADGRSAACTLRACPNQ

SEQ ID NO: 16 - protein, 36 aa, Artificial Sequence
  Description of Artificial Sequence Synthetic polypeptide
GSSCRPGRTFRDRCNTCRCGADGRSAACTLRACPNQ

SEQ ID NO: 17 - protein, 36 aa, Artificial Sequence
  Description of Artificial Sequence Synthetic polypeptide
GSSCEPGETFRDRCNTCRCGADGRSAACTLRACPNQ

SEQ ID NO: 18 - protein, 36 aa, Artificial Sequence
  Description of Artificial Sequence Synthetic polypeptide
GSSCEPGRTFEDRCNTCRCGADGRSAACTLRACPNQ

SEQ ID NO: 19 - protein, 36 aa, Artificial Sequence
  Description of Artificial Sequence Synthetic polypeptide
GSSCEPGRTFRRRCNTCRCGADGRSAACTLRACPNQ

SEQ ID NO: 20 - protein, 36 aa, Artificial Sequence
  Description of Artificial Sequence Synthetic polypeptide
GSSCEPGRTFRDECNTCRCGADGRSAACTLRACPNQ

SEQ ID NO: 21 - protein, 36 aa, Artificial Sequence
  Description of Artificial Sequence Synthetic polypeptide
GSSCEPGRTFRDRCDTCRCGADGRSAACTLRACPNQ

SEQ ID NO: 22 - protein, 36 aa, Artificial Sequence
  Description of Artificial Sequence Synthetic polypeptide
GSSCEPGRTFRDRCNTCECGADGRSAACTLRACPNQ

SEQ ID NO: 23 - protein, 36 aa, Artificial Sequence
  Description of Artificial Sequence Synthetic polypeptide
GSSCEPGRTFRDRCNTCRCGARGRSAACTLRACPNQ

SEQ ID NO: 24 - protein, 36 aa, Artificial Sequence
  Description of Artificial Sequence Synthetic polypeptide
GSSCEPGRTFRDRCNTCRCGADGESAACTLRACPNQ

SEQ ID NO: 25 - protein, 36 aa, Artificial Sequence
  Description of Artificial Sequence Synthetic polypeptide
GSSCEPGRTFRDRCNTCRCGADGRSAACTLEACPNQ

SEQ ID NO: 26 - protein, 36 aa, Artificial Sequence
  Description of Artificial Sequence Synthetic polypeptide
GSSCEPGRTFRDRCNTCKCGADGRSAACTLRACPNQ

SEQ ID NO: 27 - protein, 41 aa, Artificial Sequence
  Description of Artificial Sequence Synthetic polypeptide
GSSRRRRRRRRCEPGRTFRDRCNTCRCGADGRSAACTLRAC

SEQ ID NO: 28 - protein, 33 aa, Artificial Sequence
  Description of Artificial Sequence Synthetic polypeptide
GSSCLPNETFRFDCNSCRCNDDGRTAACTLMLC

SEQ ID NO: 29 - protein, 38 aa, Artificial Sequence
  Description of Artificial Sequence Synthetic polypeptide
GSEISCEPGKTFKDKCNTCRCGADGKSAACTLKACPNQ

SEQ ID NO: 30 - protein, 38 aa, Artificial Sequence
  Description of Artificial Sequence Synthetic polypeptide
GSEISCEPGKTFRDRCNTCRCGADGRSAACTLRACPNQ

SEQ ID NO: 31 - protein, 38 aa, Artificial Sequence
  Description of Artificial Sequence Synthetic polypeptide
GSEISCEPGRTFKDRCNTCRCGADGRSAACTLRACPNQ

SEQ ID NO: 32 - protein, 38 aa, Artificial Sequence
  Description of Artificial Sequence Synthetic polypeptide
GSEISCEPGRTFRDKCNTCRCGADGRSAACTLRACPNQ

SEQ ID NO: 33 - protein, 38 aa, Artificial Sequence
  Description of Artificial Sequence Synthetic polypeptide
GSEISCEPGRTFRDRCNTCRCGADGKSAACTLRACPNQ

SEQ ID NO: 34 - protein, 38 aa, Artificial Sequence
  Description of Artificial Sequence Synthetic polypeptide
GSEISCEPGRTFRDRCNTCRCGADGRSAACTLKACPNQ

SEQ ID NO: 35 - protein, 38 aa, Artificial Sequence; identical to SEQ ID NO: 29
  Description of Artificial Sequence Synthetic polypeptide

SEQ ID NO: 36 - protein, 36 aa, Artificial Sequence; identical to SEQ ID NO: 15
  Description of Artificial Sequence Synthetic polypeptide
  MOD_RES (1)..(36): D-amino acid

SEQ ID NO: 37 - protein, 34 aa, Artificial Sequence
  Description of Artificial Sequence Synthetic polypeptide
SCEPGRTFEDECNTCRCGADGRSAACTLEACPNQ

SEQ ID NO: 38 - protein, 34 aa, Artificial Sequence
  Description of Artificial Sequence Synthetic polypeptide
SCEPGRTFADACNTCRCGADGRSAACTLAACPNQ

SEQ ID NO: 39 - protein, 34 aa, Artificial Sequence
  Description of Artificial Sequence Synthetic polypeptide
SCEPGKTFRDRCNTCRCGADGRSAACTLRACPNQ

SEQ ID NO: 40 - protein, 34 aa, Artificial Sequence
  Description of Artificial Sequence Synthetic polypeptide
SCEPGRTFKDRCNTCRCGADGRSAACTLRACPNQ

SEQ ID NO: 41 - protein, 34 aa, Artificial Sequence
  Description of Artificial Sequence Synthetic polypeptide
SCEPGRTFRDKCNTCRCGADGRSAACTLRACPNQ

SEQ ID NO: 42 - protein, 34 aa, Artificial Sequence
  Description of Artificial Sequence Synthetic polypeptide
SCEPGRTFRDRCNTCRCGADGKSAACTLRACPNQ

SEQ ID NO: 43 - protein, 34 aa, Artificial Sequence
  Description of Artificial Sequence Synthetic polypeptide
SCEPGRTFRDRCNTCRCGADGRSAACTLKACPNQ

SEQ ID NO: 44 - protein, 33 aa, Artificial Sequence
  Description of Artificial Sequence Synthetic polypeptide
SCEPGTTFRDRCNTCRCGSDGRSAACTLRACPQ

SEQ ID NO: 45 - protein, 36 aa, Artificial Sequence
  Description of Artificial Sequence Synthetic polypeptide
SCTPGTTFRDRCNTCRCSSNGRSAACTLRACPPGSY

SEQ ID NO: 46 - protein, 36 aa, Artificial Sequence
  Description of Artificial Sequence Synthetic polypeptide
SCTPGTTFRNRCNTCRCGSNGRSASCTLMACPPGSY

SEQ ID NO: 47 - protein, 36 aa, Artificial Sequence
  Description of Artificial Sequence Synthetic polypeptide
SCTPGATFRNRCNTCRCGSNGRSASCTLMACPPGSY

SEQ ID NO: 48 - protein, 31 aa, Artificial Sequence
  Description of Artificial Sequence Synthetic polypeptide
SCQPGTTYQRGCNTCRCLEDGQTEACTLRLC

SEQ ID NO: 49 - protein, 36 aa, Artificial Sequence
  Description of Artificial Sequence Synthetic polypeptide
SCTPGATYREGCNICRCRSDGRSGACTRRICPVDSN

SEQ ID NO: 50 - protein, 32 aa, Artificial Sequence
  Description of Artificial Sequence Synthetic polypeptide
SCQPGTTFRRDCNTCVCNRDGTNAACTLRACL

SEQ ID NO: 51 - protein, 34 aa, Artificial Sequence
  Description of Artificial Sequence Synthetic polypeptide
SCEPGRTFRDRCNTCRCGADGRSAACTLRACPNQ

SEQ ID NO: 52 - protein, 34 aa, Artificial Sequence
  Description of Artificial Sequence Synthetic polypeptide
SCRPGRTFRDRCNTCRCGADGRSAACTLRACPNQ

SEQ ID NO: 53 - protein, 34 aa, Artificial Sequence
  Description of Artificial Sequence Synthetic polypeptide
SCEPGETFRDRCNTCRCGADGRSAACTLRACPNQ

SEQ ID NO: 54 - protein, 34 aa, Artificial Sequence
  Description of Artificial Sequence Synthetic polypeptide
SCEPGRTFEDRCNTCRCGADGRSAACTLRACPNQ

SEQ ID NO: 55 - protein, 34 aa, Artificial Sequence
  Description of Artificial Sequence Synthetic polypeptide
SCEPGRTFRRRCNTCRCGADGRSAACTLRACPNQ

SEQ ID NO: 56 - protein, 34 aa, Artificial Sequence
  Description of Artificial Sequence Synthetic polypeptide
SCEPGRTFRDECNTCRCGADGRSAACTLRACPNQ

SEQ ID NO: 57 - protein, 34 aa, Artificial Sequence
  Description of Artificial Sequence Synthetic polypeptide
SCEPGRTFRDRCDTCRCGADGRSAACTLRACPNQ

SEQ ID NO: 58 - protein, 34 aa, Artificial Sequence
  Description of Artificial Sequence Synthetic polypeptide
SCEPGRTFRDRCNTCECGADGRSAACTLRACPNQ

SEQ ID NO: 59 - protein, 34 aa, Artificial Sequence
  Description of Artificial Sequence Synthetic polypeptide
SCEPGRTFRDRCNTCRCGARGRSAACTLRACPNQ

SEQ ID NO: 60 - protein, 34 aa, Artificial Sequence
  Description of Artificial Sequence Synthetic polypeptide
SCEPGRTFRDRCNTCRCGADGESAACTLRACPNQ

SEQ ID NO: 61 - protein, 34 aa, Artificial Sequence
  Description of Artificial Sequence Synthetic polypeptide
SCEPGRTFRDRCNTCRCGADGRSAACTLEACPNQ

SEQ ID NO: 62 - protein, 34 aa, Artificial Sequence
  Description of Artificial Sequence Synthetic polypeptide
SCEPGRTFRDRCNTCKCGADGRSAACTLRACPNQ

SEQ ID NO: 63 - protein, 39 aa, Artificial Sequence
  Description of Artificial Sequence Synthetic polypeptide
SRRRRRRRRCEPGRTFRDRCNTCRCGADGRSAACTLRAC

SEQ ID NO: 64 - protein, 31 aa, Artificial Sequence
  Description of Artificial Sequence Synthetic polypeptide
SCLPNETFRFDCNSCRCNDDGRTAACTLMLC

SEQ ID NO: 65 - protein, 36 aa, Artificial Sequence
  Description of Artificial Sequence Synthetic polypeptide
EISCEPGKTFKDKCNTCRCGADGKSAACTLKACPNQ

SEQ ID NO: 66 - protein, 36 aa, Artificial Sequence
  Description of Artificial Sequence Synthetic polypeptide
EISCEPGKTFRDRCNTCRCGADGRSAACTLRACPNQ

SEQ ID NO: 67 - protein, 36 aa, Artificial Sequence
  Description of Artificial Sequence Synthetic polypeptide
EISCEPGRTFKDRCNTCRCGADGRSAACTLRACPNQ

SEQ ID NO: 68 - protein, 36 aa, Artificial Sequence
  Description of Artificial Sequence Synthetic polypeptide
EISCEPGRTFRDKCNTCRCGADGRSAACTLRACPNQ

SEQ ID NO: 69 - protein, 36 aa, Artificial Sequence
  Description of Artificial Sequence Synthetic polypeptide
EISCEPGRTFRDRCNTCRCGADGKSAACTLRACPNQ

SEQ ID NO: 70 - protein, 36 aa, Artificial Sequence
  Description of Artificial Sequence Synthetic polypeptide
EISCEPGRTFRDRCNTCRCGADGRSAACTLKACPNQ

SEQ ID NO: 71 - protein, 36 aa, Artificial Sequence; identical to SEQ ID NO: 65
  Description of Artificial Sequence Synthetic polypeptide

SEQ ID NO: 72 - protein, 34 aa, Artificial Sequence; identical to SEQ ID NO: 51
  Description of Artificial Sequence Synthetic polypeptide
  MOD_RES (1)..(34): D-amino acid

SEQ ID NO: 73 - protein, 38 aa, Artificial Sequence
  Description of Artificial Sequence Synthetic polypeptide
  MOD_RES (5)..(5): Any amino acid
  MOD_RES (8)..(8): Any amino acid
  MOD_RES (10)..(13): Any amino acid
  MOD_RES (18)..(18): Any amino acid
  MOD_RES (20)..(21): Any amino acid
  MOD_RES (24)..(27): Any amino acid
  MOD_RES (31)..(32): Any amino acid
  MOD_RES (34)..(38): Any amino acid
  See specification as filed for detailed description of substitutions and preferred embodiments
GSSCXPGXTXXXXCNTCXCXXDGXXXXCTLXXCXXXXX

SEQ ID NO: 74 - protein, 38 aa, Artificial Sequence; identical to SEQ ID NO: 73
  Description of Artificial Sequence Synthetic polypeptide
  MOD_RES (5)..(5): Any amino acid
  MOD_RES (8)..(8): Any amino acid
  MOD_RES (10)..(13): Any amino acid
  MOD_RES (18)..(18): Any amino acid
  MOD_RES (20)..(21): Any amino acid
  MOD_RES (24)..(27): Any amino acid
  MOD_RES (31)..(32): Any amino acid
  MOD_RES (34)..(38): Any amino acid
  See specification as filed for detailed description of substitutions and preferred embodiments

SEQ ID NO: 75 - protein, 38 aa, Artificial Sequence
  Description of Artificial Sequence Synthetic polypeptide
  MOD_RES (5)..(5): Glu or Thr
  MOD_RES (8)..(8): Arg, Thr, or Ala
  MOD_RES (10)..(10): Phe or Tyr
  MOD_RES (11)..(11): Arg or Gln
  MOD_RES (12)..(12): Asp, Arg, or Asn
  MOD_RES (13)..(13): Gly, Arg, or Asp
  MOD_RES (18)..(18): Arg, Val, or Lys
  MOD_RES (20)..(20): Leu, Gly, or Asn
  MOD_RES (21)..(21): Glu, Ser, Arg, or Ala
  MOD_RES (24)..(24): Gln, Arg, or Thr
  MOD_RES (25)..(25): Thr, Ser, or Asn
  MOD_RES (26)..(26): Glu or Ala
  MOD_RES (27)..(27): Ala or Ser
  MOD_RES (31)..(31): Arg or Met
  MOD_RES (32)..(32): Leu or Ala
  MOD_RES (34)..(34): Pro, Leu, Ser, or absent
  MOD_RES (35)..(35): Pro, Ser, or absent
  MISC_FEATURE (36)..(38): May or may not be present
  See specification as filed for detailed description of substitutions and preferred embodiments
GSSCXPGXTXXXXCNTCXCXXDGXXXXCTLXXCXXGSY

SEQ ID NO: 76 - protein, 38 aa, Artificial Sequence; identical to SEQ ID NO: 75
  Description of Artificial Sequence Synthetic polypeptide
  MOD_RES (5)..(5): Glu or Thr
  MOD_RES (8)..(8): Arg, Thr, or Ala
  MOD_RES (10)..(10): Phe or Tyr
  MOD_RES (11)..(11): Arg or Gln
  MOD_RES (12)..(12): Asp, Arg, or Asn
  MOD_RES (13)..(13): Gly, Arg, or Asp
  MOD_RES (18)..(18): Arg, Val, or Lys
  MOD_RES (20)..(20): Leu, Gly, or Asn
  MOD_RES (21)..(21): Glu, Ser, Arg, or Ala
  MOD_RES (24)..(24): Gln, Arg, or Thr
  MOD_RES (25)..(25): Thr, Ser, or Asn
  MOD_RES (26)..(26): Glu or Ala
  MOD_RES (27)..(27): Ala or Ser
  MOD_RES (31)..(31): Arg or Met
  MOD_RES (32)..(32): Leu or Ala
  MOD_RES (34)..(34): Pro, Leu, Ser, or absent
  MOD_RES (35)..(35): Pro, Ser, or absent
  MISC_FEATURE (36)..(38): May or may not be present
  See specification as filed for detailed description of substitutions and preferred embodiments

SEQ ID NO: 77 - protein, 36 aa, Artificial Sequence
  Description of Artificial Sequence Synthetic polypeptide
  MOD_RES (3)..(3): Any amino acid
  MOD_RES (6)..(6): Any amino acid
  MOD_RES (8)..(11): Any amino acid
  MOD_RES (16)..(16): Any amino acid
  MOD_RES (18)..(19): Any amino acid
  MOD_RES (22)..(25): Any amino acid
  MOD_RES (29)..(30): Any amino acid
  MOD_RES (32)..(36): Any amino acid
  See specification as filed for detailed description of substitutions and preferred embodiments
SCXPGXTXXXXCNTCXCXXDGXXXXCTLXXCXXXXX

SEQ ID NO: 78 - protein, 36 aa, Artificial Sequence; identical to SEQ ID NO: 77
  Description of Artificial Sequence Synthetic polypeptide
  MOD_RES (3)..(3): Any amino acid
  MOD_RES (6)..(6): Any amino acid
  MOD_RES (8)..(11): Any amino acid
  MOD_RES (16)..(16): Any amino acid
  MOD_RES (18)..(19): Any amino acid
  MOD_RES (22)..(25): Any amino acid
  MOD_RES (29)..(30): Any amino acid
  MOD_RES (32)..(36): Any amino acid
  See specification as filed for detailed description of substitutions and preferred embodiments

SEQ ID NO: 79 - protein, 38 aa, Artificial Sequence; identical to SEQ ID NO: 75
  Description of Artificial Sequence Synthetic polypeptide
  MOD_RES (5)..(5): Glu or Thr
  MOD_RES (8)..(8): Arg, Thr, or Ala
  MOD_RES (10)..(10): Phe or Tyr
  MOD_RES (11)..(11): Arg or Gln
  MOD_RES (12)..(12): Asp, Arg, or Asn
  MOD_RES (13)..(13): Gly, Arg, or Asp
  MOD_RES (18)..(18): Arg, Val, or Lys
  MOD_RES (20)..(20): Leu, Gly, or Asn
  MOD_RES (21)..(21): Glu, Ser, Arg, or Ala
  MOD_RES (24)..(24): Gln, Arg, or Thr
  MOD_RES (25)..(25): Thr, Ser, or Asn
  MOD_RES (26)..(26): Glu or Ala
  MOD_RES (27)..(27): Ala or Ser
  MOD_RES (31)..(31): Arg or Met
  MOD_RES (32)..(32): Leu or Ala
  MOD_RES (34)..(34): Pro, Leu, Ser, or absent
  MOD_RES (35)..(35): Pro, Ser, or absent
  MISC_FEATURE (36)..(38): May or may not be present
  See specification as filed for detailed description of substitutions and preferred embodiments

SEQ ID NO: 80 - protein, 38 aa, Artificial Sequence; identical to SEQ ID NO: 75
  Description of Artificial Sequence Synthetic polypeptide
  MOD_RES (5)..(5): Glu or Thr
  MOD_RES (8)..(8): Arg, Thr, or Ala
  MOD_RES (10)..(10): Phe or Tyr
  MOD_RES (11)..(11): Arg or Gln
  MOD_RES (12)..(12): Asp, Arg, or Asn
  MOD_RES (13)..(13): Gly, Arg, or Asp
  MOD_RES (18)..(18): Arg, Val, or Lys
  MOD_RES (20)..(20): Leu, Gly, or Asn
  MOD_RES (21)..(21): Glu, Ser, Arg, or Ala
  MOD_RES (24)..(24): Gln, Arg, or Thr
  MOD_RES (25)..(25): Thr, Ser, or Asn
  MOD_RES (26)..(26): Glu or Ala
  MOD_RES (27)..(27): Ala or Ser
  MOD_RES (31)..(31): Arg or Met
  MOD_RES (32)..(32): Leu or Ala
  MOD_RES (34)..(34): Pro, Leu, Ser, or absent
  MOD_RES (35)..(35): Pro, Ser, or absent
  MISC_FEATURE (36)..(38): May or may not be present
  See specification as filed for detailed description of substitutions and preferred embodiments

SEQ ID NO: 81 - protein, 35 aa, Artificial Sequence
  Description of Artificial Sequence Synthetic polypeptide
EVTCEPGTTFKDKCNTCRCGSDGKSAACTLKACPQ

SEQ ID NO: 82 - protein, 36 aa, Artificial Sequence; identical to SEQ ID NO: 65
  Description of Artificial Sequence Synthetic polypeptide

SEQ ID NO: 83 - protein, 35 aa, Artificial Sequence
  Description of Artificial Sequence Synthetic polypeptide
AGECTPGQTKKQDCNTCTCTPTGIWGCTRKACRTT

SEQ ID NO: 84 - protein, 4 aa, Artificial Sequence
  Description of Artificial Sequence Synthetic peptide
GAGA

(19) United States
(12) Patent Application Publication
(10) Pub. No.: US 2019/0292243 A1
(43) Pub. Date: Sep. 26, 2019

(54) PEPTIDES AND METHODS OF USE THEREOF

(71) Applicant: Fred Hutchinson Cancer Research Center, Seattle, WA (US)

(72) Inventors: James OLSON, Seattle, WA (US); Theo SOTTERO, Seattle, WA (US); Emily GIRARD, Renton, WA (US); Andrew MHYRE, Kenmore, WA (US); Colin CORRENTI, Seattle, WA (US); Christopher MEHLIN, Seattle, WA (US); Mesfin Mulugeta GEWE, Bothell, WA (US); Gene Gregory HOPPING, Seattle, WA (US)

(21) Appl. No.: 16/326,154

(22) PCT Filed: Aug. 22, 2017
(86) PCT No.: PCT/US17/48084

§ 371 Date: Feb. 15, 2019

Related U.S. Application Data
(60) Provisional application No. 62/378,172, filed on Aug. 22, 2016.

(51) Int. Cl. C07K 14/81; A61K 47/55; A61K 47/64; A61K 49/00; A61K 51/08; A61P 35/00

(52) U.S. Cl. CPC C07K 14/811; A61K 47/55; A61P 35/00; A61K 49/0056; A61K 51/08; A61K 47/64

(57) ABSTRACT

Peptides that home, migrate to, distribute to, accumulate in, are directed to, and/or bind to tumors, cancerous tissues and cells thereof are disclosed. Pharmaceutical compositions and uses for peptides, peptide-active agent complexes comprising such peptides, or peptide-detectable agent complexes comprising such peptides are additionally disclosed. Such compositions can be formulated for targeted or untargeted delivery of a drug to a target region, tissue, structure or cell. Targeted compositions of the disclosure can deliver peptide, peptide-active agent complexes, or peptide-detectable agent complexes to target regions, tissues, structures or cells targeted by the peptide.

### CROSS REFERENCE

This application claims the benefit of U.S. Provisional Patent Application No. 62/378,172, filed Aug. 22, 2016, the entire contents of which are incorporated by reference.

### STATEMENT AS TO FEDERALLY SPONSORED RESEARCH

This invention was made with government support under CA135491 awarded by the National Institutes for Health. The government has certain rights to the invention.

### SEQUENCE LISTING

The instant application contains a Sequence Listing which has been submitted electronically in ASCII format and is hereby incorporated by reference in its entirety. Said ASCII copy, created on Sep. 5, 2017, is named 44189-714_601_SL.txt and is 46,478 bytes in size.

### BACKGROUND

For many types of cancers, patient prognosis is influenced by drug efficacy and surgical access to the tumor. Cancers such as sarcomas and solid tumors are particularly hard to treat due to the difficulty of delivering efficacious doses of drug to cancerous tissues and cells thereof, while minimizing the level of off-target, negative side effects of the drug in other tissues. Consequently, there is a need for targeting drugs to cancerous tissues and cells thereof to achieve localized delivery of an efficacious dose of drug, while minimizing off-target negative effects in other tissues. Typical cancer drug regimens are often limited by dose-limiting toxicities, and although some antibody-drug conjugates are used to target drugs to specific tumors in order to limit off-target toxicity, such specific therapies are not available for many cancers. Additionally, the precision of tumor resection is dependent on intra-operative imaging to detect tumor margins or small foci of cancer cells, and current methods of intra-operative imaging of cancerous tissues are imprecise. Herein, we provide new peptides that target cancer.

### SUMMARY

In various aspects, the present disclosure provides a peptide conjugate, wherein the peptide comprises a serine protease inhibitor and wherein the serine protease inhibitor is conjugated to an active agent, a detectable agent, or a combination thereof.

In some aspects, the serine protease inhibitor is a pacifastin family member. In some aspects, the pacifastin family member is a Locusta migratoria chymotrypsin inhibitor II (LCMI-II).

In some aspects, the peptide comprises a sequence of any one of SEQ ID NO: 73-SEQ ID NO: 80, or a fragment thereof.

In some aspects, the peptide comprises a sequence that has at least 75%, at least 80%, at least 85%, at least 90%, at least 92%, at least 95%, at least 97%, or at least 99% sequence identity with any one of SEQ ID NO: 1-SEQ ID NO: 35 or a fragment thereof. In some aspects, the sequence is any one of SEQ ID NO: 1-SEQ ID NO: 35 or a fragment thereof.

In some aspects, the peptide comprises a sequence that has at least 75%, at least 80%, at least 85%, at least 90%, at least 92%, at least 95%, at least 97%, or at least 99% sequence identity with any one of SEQ ID NO: 37-SEQ ID NO: 71 or a fragment thereof. In some aspects, the sequence is any one of SEQ ID NO: 37-SEQ ID NO: 71 or a fragment thereof.

In some aspects, the peptide is a knotted peptide. In some aspects, the knotted peptide comprises or is derived from a human protein or peptide.

In some aspects, the peptide comprises at least 6, at least 8, at least 10, at least 12, at least 14, or at least 16 cysteine residues. In some aspects, the peptide comprises three or more disulfide bridges formed between cysteine residues, wherein one of the disulfide bridges passes through a loop formed by two other disulfide bridges. In some aspects, the peptide comprises a plurality of disulfide bridges formed between cysteine residues. In some aspects, at least 5% or more of the residues are cysteines forming intramolecular disulfide bonds. In some aspects, the peptide comprises a disulfide through a disulfide knot.

In some aspects, at least one amino acid residue of the peptide is in an L configuration, or wherein at least one amino acid residue of the peptide is in a D configuration.

In some aspects, the peptide comprises a sequence at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues long.

In some aspects, the peptide is arranged in a multimeric structure with at least one other peptide. In some aspects, the multimeric structure comprises a dimer, trimer, tetramer, pentamer, hexamer, or heptamer.

In some aspects, the peptide has a positive net charge greater than +0.5 at physiological pH. In some aspects, the peptide has a negative net charge lower than −0.5 at physiological pH.

In some aspects, the peptide comprises an isoelectric point less than or equal to about 7.5. In some aspects, the peptide comprises an isoelectric point greater than or equal to about 7.5. In some aspects, the peptide comprises an isoelectric point within a range from about 3.0 to about 10.0.

In some aspects, the peptide comprises a non-uniform charge distribution. In some aspects, the peptide comprises one or more regions of concentrated positive charge. In some aspects, the peptide comprises one or more regions of concentrated negative charge.

In some aspects, the peptide comprises a mass-average molecular weight (Mw) less than or equal to 6 kDa, less than or equal to about 50 kDa, or less than or equal to about 60 kDa. In some aspects, the peptide comprises a mass-average molecular weight (Mw) within a range from about 0.5 kDa to about 50 kDa, or within a range from about 0.5 kDa to about 60 kDa.

In some aspects, the peptide is stable at pH values greater than or equal to about 7.0. In some aspects, the peptide is stable at pH values less than or equal to about 5.0, less than or equal to about 3.0, or within a range from about 3.0 to about 5.0. In some aspects, the peptide is stable at pH values within a range from about 5.0 to about 7.0. In some aspects, the peptide being stable comprises one or more of: the peptide being capable of performing its therapeutic effect, the peptide being soluble, the peptide being resistant to protease degradation, the peptide being resistant to reduction, the peptide being resistant to pepsin degradation, the peptide being resistant to trypsin degradation, the peptide being reduction resistant, or the peptide being resistant to an elevated temperature.

In some aspects, upon administration to a subject, the peptide homes, targets, migrates to, distributes to, accumulates in, or is directed to a specific region, tissue, structure, or cell of the subject.

In some aspects, at least one residue of the peptide comprises a chemical modification. In some aspects, the chemical modification is blocking the N-terminus of the peptide. In some aspects, the chemical modification is methylation, acetylation, or acylation. In some aspects, the chemical modification is: i) methylation of one or more lysine residues or analog thereof; ii) methylation of an N-terminus; or iii) methylation of one or more lysine residue or analog thereof and methylation of the N-terminus. In some aspects, the peptide is linked to an acyl adduct.

In some aspects, the active agent is fused with the peptide at an N-terminus or a C terminus. In some aspects, the active agent is an antibody, antibody fragment, or single chain Fv. In some aspects, the active agent is an Fc domain. In some aspects, the peptide fused with an Fc domain comprises a contiguous sequence. In some aspects, 1, 2, 3, 4, 5, 6, 7, 8, 9, or 10 active agents are linked to the peptide.

In some aspects, the peptide is linked to the active agent via a cleavable linker.

In some aspects, the peptide is linked to the active agent at an N-terminus, at the epsilon amine of an internal lysine residue, at the carboxylic acid of an asparagine or glutamine residue, or a C-terminus of the peptide by a linker. In some aspects, the peptide further comprises a non-natural amino acid, wherein the non-natural amino acid is an insertion, appendage, or substitution for another amino acid. In some aspects, the peptide is linked to the active agent at the non-natural amino acid by a linker. In some aspects, the linker comprises an amide bond, an ester bond, a carbamate bond, a carbonate bond, a hydrazine bond, an oxime bond, a disulfide bond, a thioester bond, a thioether bond or a carbon-nitrogen bond. In some aspects, the cleavable linker comprises a cleavage site for matrix metalloproteinases, thrombin, cathepsins, peptidases, or beta-glucuronidase.

In some aspects, the linker is a hydrolytically labile linker.

In some aspects, the peptide is linked to the active agent via a non-cleavable linker.

In some aspects, the active agent is selected from the group consisting of: a peptide, an oligopeptide, a polypeptide, a polynucleotide, a polyribonucleotide, a DNA, a cDNA, a ssDNA, a RNA, a dsRNA, a micro RNA, an oligonucleotide, an antibody, a single chain variable fragment (scFv), an antibody fragment, a cytokine, a hormone, a growth factor, a checkpoint inhibitor, an immune modulator, a neurotransmitter, a chemical agent, a cytotoxic molecule, a toxin, a radiosensitizer, a radioprotectant, a therapeutic small molecule, a nanoparticle, a liposome, a polymer, a dendrimer, an enzyme, a chemokine, a chemical agent, a fatty acid, a peptidomimetic, a complement fixing peptide or protein, polyethylene glycol, a lipid, an Fc region, a metal, a metal chelate, a steroid, a corticosteroid, an anti-inflammatory agent, an immunosuppressant, a protease inhibitor, an amino sugar, a chemotherapeutic, a cytotoxic chemical, a tyrosine kinase inhibitor, an anti-infective agent, an antibiotic, an anti-viral agent, an anti-fungal agent, an aminoglycoside, a nonsteroidal anti-inflammatory drug (NSAID), a statin, a biopolymer, a polysaccharide, a proteoglycan, an immunomodulatory agent, a T cell activating agent, a macrophage activating agent, a natural killer cell activating agent, or a glycosaminoglycan. In some aspects, the active agent inhibits a protease, has antimicrobial activity, has anticancer activity, has anti-inflammatory activity, or a combination thereof. In some aspects, the active agent is a chemotherapeutic agent. In some aspects, the cytotoxic molecule is an auristatin, a maytansinoid, MMAE, DM1, DM4, doxorubicin, a calicheamicin, a platinum compound, cisplastin, a taxane, paclitaxel, a BACE inhibitor, a Bcl-xL inhibitor, WEHI-539, venetoclax, ABT-199, navitoclax, AT-101, obatoclax, a pyrrolobenzodiazepine, pyrrolobenzodiazepine dimer, or dolastatin. In some aspects, the active agent is a knotted peptide. In some aspects, the active agent is a radiosensitizer or photosensitizer.

In some aspects, the detectable agent is fused with the peptide at an N-terminus or a C-terminus of the peptide. In some aspects, 1, 2, 3, 4, 5, 6, 7, 8, 9, or 10 detectable agents are linked to the peptide.

In some aspects, the peptide is linked to the detectable agent via a cleavable linker.

In some aspects, the peptide is linked to the detectable agent at an N-terminus, at the epsilon amine of an internal lysine residue, at the carboxylic acid of an asparagine or glutamine residue, or a C-terminus of the peptide by a linker. In some aspects, the peptide further comprises a non-natural amino acid, wherein the non-natural amino acid is an insertion, appendage, or substitution for another amino acid. In some aspects, the peptide is linked to the detectable agent at the non-natural amino acid by a linker. In some aspects, the linker comprises an amide bond, an ester bond, a carbamate bond, a hydrazine bond, an oxime bond, a thioether, or a carbon-nitrogen bond. In some aspects, the cleavable linker comprises a cleavage site for matrix metalloproteinases, thrombin, cathepsins, peptidases, or beta-glucuronidase.

In some aspects, the peptide is linked to the detectable agent via a non-cleavable linker.

In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In some aspects, the detectable agent is a fluorescent dye.

In some aspects, the peptide homes, targets, migrates to, distributes to, accumulates in, or is directed to a tumor or cancerous cell. In some aspects, the tumor is a solid tumor. In some aspects, the peptide, active agent, detectable agent, or combination thereof penetrates the solid tumor.

In some aspects, the peptide, active agent, detectable agent, or combination thereof is internalized into or penetrates into a cancer cell. In some aspects, the tumor or cancerous cell is from a sarcoma, cervical cancer, B cell lymphoma, breast cancer, brain cancer, Ewing sarcoma, Burkitt's lymphoma, medulloblastoma, rhabdomyosarcoma, or colorectal cancer.

In some aspects, the peptide conjugate further comprises a half-life modifying agent coupled to the peptide. In some aspects, the half-life modifying agent comprises a polymer, a polyethylene glycol (PEG), a hydroxyl starch, polyvinyl alcohol, a water soluble polymer, a zwitterionic water soluble polymer, a water soluble poly(amino acid), a water soluble polymer of proline, alanine, and serine, a water soluble polymer containing glycine, glutamic acid, and serine, an Fc region, a fatty acid, palmitic acid, or a molecules that binds to albumin.

In some aspects, the peptide is fucosylated. In some aspects, the peptide comprises SEQ ID NO: 1-SEQ ID NO: 26 or SEQ ID NO: 28-SEQ ID NO: 35 and wherein the peptide is fucosylated at Threonine-9. In some aspects, the peptide comprises SEQ ID NO: 37-SEQ ID NO: 62 or SEQ ID NO: 64-SEQ ID NO: 71 and wherein the peptide is fucosylated at Threonine-7.

In various aspects, the present disclosure provides a peptide comprises a sequence of any one of SEQ ID NO: 73-SEQ ID NO: 80, or a fragment thereof.

In various aspects, the present disclosure provides a peptide comprises a sequence that has at least 75%, at least 80%, at least 85%, at least 90%, at least 92%, at least 95%, at least 97%, or at least 99% sequence identity with any one of SEQ ID NO: 1-SEQ ID NO: 34 or a fragment thereof. In some aspects, the sequence is any one of SEQ ID NO: 1-SEQ ID NO: 34 or a fragment thereof.

In various aspects, the present disclosure provides a peptide comprises a sequence that has at least 75%, at least 80%, at least 85%, at least 90%, at least 92%, at least 95%, at least 97%, or at least 99% sequence identity with any one of SEQ ID NO: 37-SEQ ID NO: 70 or a fragment thereof. In some aspects, the sequence is any one of SEQ ID NO: 37-SEQ ID NO: 70 or a fragment thereof.

In some aspects, the peptide is a knotted peptide. In some aspects, the knotted peptide comprises or is derived from a human protein or peptide. In some aspects, the peptide comprises at least 6, at least 8, at least 10, at least 12, at least 14, or at least 16 cysteine residues. In some aspects, the peptide comprises three or more disulfide bridges formed between cysteine residues, wherein one of the disulfide bridges passes through a loop formed by two other disulfide bridges. In some aspects, the peptide comprises a plurality of disulfide bridges formed between cysteine residues. In some aspects, at least 5% or more of the residues are cysteines forming intramolecular disulfide bonds. In some aspects, the peptide comprises a disulfide through a disulfide knot.

In some aspects, at least one amino acid residue of the peptide is in an L configuration, or wherein at least one amino acid residue of the peptide is in a D configuration. In some aspects, the peptide comprises a sequence at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues long.

In some aspects, the peptide is arranged in a multimeric structure with at least one other peptide. In some aspects, the multimeric structure comprises a dimer, trimer, tetramer, pentamer, hexamer, or heptamer.

In some aspects, the peptide has a positive net charge greater than +0.5 at physiological pH. In some aspects, the peptide has a negative net charge lower than −0.5 at physiological pH.

In some aspects, the peptide comprises an isoelectric point less than or equal to about 7.5. In some aspects, the peptide comprises an isoelectric point greater than or equal to about 7.5. In some aspects, the peptide comprises an isoelectric point within a range from about 3.0 to about 10.0.

In some aspects, the peptide comprises a non-uniform charge distribution. In some aspects, the peptide comprises one or more regions of concentrated positive charge. In some aspects, the peptide comprises one or more regions of concentrated negative charge.

In some aspects, the peptide comprises a mass-average molecular weight (Mw) less than or equal to 6 kDa, less than or equal to about 50 kDa, or less than or equal to about 60 kDa. In some aspects, the peptide comprises a mass-average molecular weight (Mw) within a range from about 0.5 kDa to about 50 kDa, or within a range from about 0.5 kDa to about 60 kDa.

In some aspects, the peptide is stable at pH values greater than or equal to about 7.0. In some aspects, the peptide is stable at pH values less than or equal to about 5.0, less than or equal to about 3.0, or within a range from about 3.0 to about 5.0. In some aspects, the peptide is stable at pH values within a range from about 5.0 to about 7.0.

In some aspects, the peptide being stable comprises one or more of: the peptide being capable of performing its therapeutic effect, the peptide being soluble, the peptide being resistant to protease degradation, the peptide being resistant to reduction, the peptide being resistant to pepsin degradation, the peptide being resistant to trypsin degradation, the peptide being reduction resistant, or the peptide being resistant to an elevated temperature.

In some aspects, upon administration to a subject, the peptide homes, targets, migrates to, distributes to, accumulates in, or is directed to a specific region, tissue, structure, or cell of the subject.

In some aspects, at least one residue of the peptide comprises a chemical modification. In some aspects, the chemical modification is blocking the N-terminus of the peptide. In some aspects, the chemical modification is methylation, acetylation, or acylation. In some aspects, the chemical modification is: i) methylation of one or more lysine residues or analog thereof; ii) methylation of an N-terminus; or iii) methylation of one or more lysine residue or analog thereof and methylation of the N-terminus. In some aspects, the peptide is linked to an acyl adduct.

In some aspects, the peptide homes, targets, migrates to, distributes to, accumulates in, or is directed to a tumor or cancerous cell. In some aspects, the tumor is a solid tumor. In some aspects, the peptide or active agent penetrates the solid tumor.

In some aspects, the peptide is internalized into or penetrates into a cancer cell.

In some aspects, the tumor or cancerous cell is from a sarcoma, cervical cancer, B cell lymphoma, breast cancer, brain cancer, Ewing sarcoma, Burkitt's lymphoma, medulloblastoma, rhabdomyosarcoma, or colorectal cancer.

In some aspects, the peptide is coupled to a half-life modifying agent. In some aspects, the half-life modifying agent comprises a polymer, a polyethylene glycol (PEG), a hydroxyl starch, polyvinyl alcohol, a water soluble polymer, a zwitterionic water soluble polymer, a water soluble poly(amino acid), a water soluble polymer of proline, alanine, and serine, a water soluble polymer containing glycine, glutamic acid, and serine, an Fc region, a fatty acid, palmitic acid, or a molecules that binds to albumin.

In some aspects, the peptide is fucosylated. In some aspects, the peptide comprises SEQ ID NO: 1-SEQ ID NO: 26 or SEQ ID NO: 28-SEQ ID NO: 35 and wherein the peptide is fucosylated at Threonine-9. In some aspects, the peptide comprises SEQ ID NO: 37-SEQ ID NO: 62 or SEQ ID NO: 64-SEQ ID NO: 71 and wherein the peptide is fucosylated at Threonine-7.

In some aspects, any peptide of any peptide conjugate of this disclosure or any peptide of this disclosure is peptide that is a non-naturally occurring peptide.

In various aspects, the present disclosure provides a pharmaceutical composition comprises any peptide conjugate of this disclosure, any peptide of this disclosure or a salt thereof, and a pharmaceutically acceptable carrier. In some aspects, the pharmaceutical composition is formulated for administration to a subject. In some aspects, the pharmaceutical composition is formulated for inhalation, intranasal administration, oral administration, topical administration, intravenous administration, subcutaneous administration, intra-articular administration, intramuscular administration, intrathecal administration, intraperitoneal administration, or a combination thereof.

In various aspects, the present disclosure provides a method of treating a condition in a subject in need thereof comprises administering to the subject any peptide conjugate of this disclosure, any peptide of this disclosure, or any pharmaceutical composition of this disclosure. In some aspects, the peptide conjugate, the peptide, or pharmaceutical composition is administered by inhalation, intranasally, orally, topically, intravenously, subcutaneously, intraarticularly, intramuscularly, intrathecally, intraperitoneally, or a combination thereof. In some aspects, the peptide conjugate, the peptide, or pharmaceutical composition homes, targets, migrates to, distributes to, accumulates in, or is directed to a cancerous or diseased region, tissue, structure, or cell of the subject following administration.

In some aspects, the condition is a tumor or cancer. In some aspects, the condition is a solid tumor. In some aspects, the condition is a metastatic cancer. In some aspects, the condition is a sarcoma, cervical cancer, B cell lymphoma, breast cancer, brain cancer, Ewing sarcoma, Burkitt's lymphoma, medulloblastoma, rhabdomyosarcoma, or colorectal cancer.

In some aspects, the method is combined with other treatments. In some aspects, the other treatments comprise chemotherapy, radiation therapy, or immunomodulatory therapy.

In various aspects, the present disclosure provides a method of imaging an organ or body region of a subject comprises administering to the subject any peptide conjugate of the disclosure, any peptide of the disclosure, or any pharmaceutical composition of the disclosure; and imaging the organ or body region of the subject.

In some aspects, the method further comprises detecting a cancer or diseased region, tissue, structure or cell of the subject.

In some aspects, the method further comprises performing surgery on the subject.

In some aspects, the method further comprises treating the cancer.

In some aspects, the surgery comprises removing the cancer or the diseased region, tissue, structure or cell of the subject.

In some aspects, the method further comprises imaging the cancer or diseased region, tissue, structure, or cell of the subject after surgical removal.

In various aspects, the present disclosure provides a method of making any peptide of the peptide conjugate of this disclosure or any peptide of this disclosure comprises making the peptide by recombinant expression.

In various aspects, the present disclosure provides a method of making any peptide of the peptide conjugate of this disclosure or any peptide of this disclosure comprises making the peptide by chemical synthesis.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a knottin chymotrypsin inhibitor II, the sequence of chymotrypsin inhibitor II, and the sequences of two variants of chymotrypsin inhibitor II.

FIG. 1A illustrates a knottin chymotrypsin inhibitor II (Boigegrain et al., Biochem Biophys Res Commun., 189(2):790-3 (1992)). C term indicates the C-terminal end of the peptide.

FIG. 1B shows a sequence comparison of sequence of chymotrypsin inhibitor II (CTI) (SEQ ID NO: 71: EISCEPGKTFKDKCNTCRCGADGKSAACTLKACPNQ) with a peptide of SEQ ID NO: 15 and a peptide of SEQ ID NO: 26.

FIG. 2 shows nonreduced and reduced bands of a SEQ ID NO: 15 peptide on SDS-PAGE gels.

FIG. 3 shows HPLC chromatograms and sequence comparisons of SEQ ID NO: 15 peptide variants.

FIG. 3A shows HPLC chromatograms of a peptide of SEQ ID NO: 9, illustrating an example of small-scale expression of the peptide of this disclosure.

FIG. 3B shows HPLC chromatograms of a peptide of SEQ ID NO: 21, illustrating an example of small-scale expression of the peptides of this disclosure.

FIG. 3C shows HPLC chromatograms of a peptide of SEQ ID NO: 28, illustrating an example of small-scale expression of the peptides of this disclosure.

FIG. 3D shows a sequence comparison of SEQ ID NO: 13, SEQ ID NO: 12, SEQ ID NO: 14, SEQ ID NO: 15, SEQ ID NO: 8, SEQ ID NO: 9, SEQ ID NO: 10, and SEQ ID NO: 11, all with the GS amino acids at the N-terminus.

FIG. 4 shows HPLC chromatograms of SEQ ID NO: 15 peptide.

FIG. 4A shows an HPLC chromatogram of a peptide of SEQ ID NO: 15.

FIG. 4B shows an HPLC chromatogram of a peptide of SEQ ID NO: 15 shown in FIG. 4A conjugated to AlexaFluor647 (AF647) (SEQ ID NO: 15-A).

FIG. 5 shows fluorescence of peptide after entering cells HeLa cells under various conditions.

FIG. 5A shows a fluorescence image taken at 40× of SEQ ID NO: 15 peptide conjugated to AlexaFluor647 (SEQ ID NO: 15-A) in red and HIV-Tat-FITC in green, indicating SEQ ID NO: 15-A peptide conjugate and HIV-Tat-FITC enter HeLa cells in different vesicles four hours after treatment.

FIG. 5B illustrates inhibition of uptake of SEQ ID NO: 15 peptide conjugated to AlexaFluor647 (SEQ ID NO: 15-A) following pre-treatment of HeLa cells with endocytosis inhibitors.

FIG. 5C illustrates inhibition of uptake of SEQ ID NO: 15 peptide conjugated to AlexaFluor647 (SEQ ID NO: 15-A) relative to dextran (a fluid-phase marker) and HIV-Tat following pre-treatment of HeLa cells with 2 mM methyl-beta cyclodextrin.

FIG. 6 shows accumulation of SEQ ID NO: 15 peptide conjugated to AlexaFluor647 (SEQ ID NO: 15-A) in Ramos lymphoma tumor tissue.

FIG. 6A shows distribution and accumulation of SEQ ID NO: 15 peptide conjugated to AlexaFluor647 (SEQ ID NO: 15-A) in a dissociated tumor 4 hours following intravenous administration in a Ramos lymphoma tumor-bearing female Harlan athymic nude mouse. Control group (left) shows tumor tissue autofluorescence after administration of sterile water as a negative control and SEQ ID NO: 15-A was administered at 10 nmol (middle) or 53 nmol doses (right).

FIG. 6B shows flow cytometry of single cell suspensions derived from dissociated tumor tissues corresponding to FIG. 6A illustrating fluorescence in the negative control (left peak; dashed line), the 10 nmol of SEQ ID NO: 15 peptide conjugated to AlexaFluor647 (SEQ ID NO: 15-A) (middle peak; dark line) dose, and the 53 nmol of SEQ ID NO: 15-A (right peak; light line) dose.

FIG. 6C shows quantification of the relative mean fluorescence intensity (MFI) from flow cytometry data shown in FIG. 6B in the negative control, the 10 nmol of SEQ ID NO: 15 peptide conjugated to AlexaFluor647 (SEQ ID NO: 15-A) dose, and the 53 nmol of SEQ ID NO: 15-A dose.

FIG. 7 shows fluorescent images of in vivo biodistribution of SEQ ID NO: 15 peptide conjugated to AlexaFluor647 (SEQ ID NO: 15-A) in two different female Harlan athymic mice bearing A673 flank tumor xenograft 4 hours after administering 10 nmol of SEQ ID NO: 15-A.

FIG. 7A shows a fluorescence image illustrating in vivo biodistribution of SEQ ID NO: 15 peptide conjugated to AlexaFluor647 (SEQ ID NO: 15-A) in a female Harlan athymic mouse bearing A673 flank tumor xenografts 4 hours after administering 10 nmol of SEQ ID NO: 15-A. Organs visualized in this image include liver (Lv), tumor (Tm), kidney (Kd), and heart (Ht).

FIG. 7B shows a whole body fluorescence image illustrating in vivo biodistribution of SEQ ID NO: 15 peptide conjugated to AlexaFluor647 (SEQ ID NO: 15-A) in a female Harlan athymic mouse different than the mouse shown in FIG. 7A, bearing A673 flank tumor xenografts 4 hours after administrating 10 nmol of SEQ ID NO: 15-A. Organs visualized in this image include liver (Lv), tumor (Tm), kidney (Kd), bladder (Bl), and heart (Ht).

FIG. 8 shows ex vivo fluorescence images illustrating biodistribution and accumulation of SEQ ID NO: 15 peptide conjugated to AlexaFluor647 (SEQ ID NO: 15-A) 4 hours after administration of 10 nmol SEQ ID NO: 15-A to a female Harlan athymic mouse bearing an A673 Ewing's sarcoma flank tumor.

FIG. 8A shows an ex vivo fluorescence image illustrating biodistribution and accumulation of SEQ ID NO: 15 peptide conjugated to AlexaFluor647 (SEQ ID NO: 15-A), 4 hours after administration of 10 nmol SEQ ID NO: 15-A to a female Harlan athymic mouse bearing an A673 Ewing's sarcoma flank tumor, in ten organs including tumor, kidney, liver, heart, tumor-draining lymph nodes (TDLN) and lumbar lymph nodes (LLN).

FIG. 8B shows an ex vivo fluorescence image illustrating biodistribution and accumulation of SEQ ID NO: 15 peptide conjugated to AlexaFluor647 (SEQ ID NO: 15-A), 4 hours after administration of 10 nmol SEQ ID NO: 15-A to a female Harlan athymic mouse bearing an A673 Ewing's sarcoma flank tumor different from that of FIG. 8A, in various organs including tumor, kidney, liver, heart, tumor-draining lymph nodes (TDLN) and lumbar lymph nodes (LLN).

FIG. 8C shows quantification of the average radiant efficiency in tumor tissues ex vivo after administration of a 0 nmol negative control, 10 nmol AlexaFluor647 (AF647), and 10 nmol SEQ ID NO: 15 peptide conjugated to AlexaFluor647 (SEQ ID NO: 15-A) peptide conjugate 4 hours after administration to a female Harlan athymic mouse bearing an A673 Ewing's sarcoma flank tumors.

FIG. 9 shows ex vivo fluorescence images illustrating biodistribution and accumulation SEQ ID NO: 15 peptide conjugated to AlexaFluor647 (SEQ ID NO: 15-) 4 hours after administration of 10 nmol SEQ ID NO: 15-A to a female Harlan athymic mouse bearing an A673 Ewing's sarcoma flank tumor.

FIG. 9A shows an ex vivo fluorescence image illustrating biodistribution and accumulation SEQ ID NO: 15 peptide conjugated to AlexaFluor647 (SEQ ID NO: 15-A), 4 hours after administration of 10 nmol SEQ ID NO: 15-A to a female Harlan athymic mouse bearing an A673 Ewing's sarcoma flank tumor, in ten organs including tumor, liver, heart, tumor-draining lymph nodes (TDLN) and lumbar lymph nodes (LLN).

FIG. 9B shows an ex vivo fluorescence image illustrating biodistribution and accumulation of SEQ ID NO: 15 peptide conjugated to AlexaFluor647 (SEQ ID NO: 15-A), 4 hours after administration of 10 nmol SEQ ID NO: 15-A to a female Harlan athymic mouse bearing an A673 Ewing's sarcoma flank tumor different from that of FIG. 10A, in various organs including tumor, liver, heart, tumor-draining lymph nodes (TDLN) and lumbar lymph nodes (LLN).

FIG. 10 illustrates SEQ ID NO: 26 peptide distribution in a mouse.

FIG. 10A shows an autoradiographic image in which the ¹⁴C signal identifies the peptide distribution in the tissues, including the RH-28 tumor of a mouse 24 hours after administration of 12 nmol radiolabeled SEQ ID NO: 26 peptide (SEQ ID NO: 26-r).

FIG. 10B shows an autoradiographic image in which the ¹⁴C signal identifies the peptide distribution in the tissues, including the RH-28 tumor of a mouse 24 hours after administration 12 nmol of the radiolabeled SEQ ID NO: 26 peptide conjugated to AF647 (SEQ ID NO: 26-Ar).

FIG. 10C shows quantification of ¹⁴C signal from radiolabeled SEQ ID NO: 26 peptide (SEQ ID NO: 26-r) and radiolabeled SEQ ID NO: 26 conjugated to AlexaFluor647 (SEQ ID NO: 26-Ar) in various tissues including skeletal muscle, tumor, liver, kidney medulla, and kidney cortex, 24 hours after either administration of 12 nmol SEQ ID NO: 26-r or 12 nmol SEQ ID NO: 26-Ar to mice bearing an RH-28 tumor.

FIG. 11 shows the distribution of radiolabeled peptide of SEQ ID NO: 26 conjugated to monomethyl auristatin E (MMAE) by a Val-Cit-PAB linker (SEQ ID NO: 26-B) after administration to a mouse and Caspase-3 staining of tissues after either administration of MMAE or SEQ ID NO: 26-Br peptide conjugate.

FIG. 11A shows a white light image of a frozen section of a mouse bearing an RH-28 tumor 24 hours after administration of 14 nmol of radiolabeled peptide of SEQ ID NO: 26 conjugated to monomethyl auristatin E (MMAE) by a Val-Cit-PAB linker (SEQ ID NO: 26-Br).

FIG. 11B shows an autoradiographic image corresponding to FIG. 11A in which the ¹⁴C signal identifies the peptide distribution in the tissues, including the RH-28 tumor, of a mouse 24 hours after administration of 14 nmol radiolabeled peptide of SEQ ID NO: 26 conjugated to monomethyl auristatin E (MMAE) by a Val-Cit-PAB linker (SEQ ID NO: 26-Br).

FIG. 11C shows Caspase-3 staining in tumor (left column), liver (middle column), and kidney (right column) in which the Caspase-3 staining identifies Caspase-3 activation in tissues, including the Ramos tumor, of a mouse 48 hours after a single systemic administration of 7.5 nmol MMAE (top row) or 7.5 nmol radiolabeled peptide of SEQ ID NO: 26 conjugated to monomethyl auristatin E (MMAE) by a Val-Cit-PAB linker (SEQ ID NO: 26-Br) (bottom row).

FIG. 11D shows a magnification of Caspase-3 staining in Ramos tumor tissues corresponding to FIG. 11C (bottom left) in which the Caspase-3 staining identifies Caspase-3 activation in tumor tissues of a mouse 48 hours after a single systemic administration of 7.5 nmol SEQ ID NO: 26 conjugated to monomethyl auristatin E (MMAE) by a Val-Cit-PAB linker (SEQ ID NO: 26-B).

FIG. 12 illustrates relative viability of HeLa cells treated for 48 hours with various doses of MMAE SEQ ID NO: 26 peptide, and SEQ ID NO: 26 peptide conjugated to MMAE by a Val-Cit-PAB linker (SEQ ID NO: 26-B).

FIG. 13 illustrates relative viability of RH28 or A673 sarcoma cell lines after 72 hours of continuous treatment of various doses of MMAE or SEQ ID NO: 26 peptide conjugated to MMAE by a Val-Cit-PAB linker (SEQ ID NO: 26-B).

FIG. 14 illustrates relative viability of A673, A204, and RH28 sarcoma cell lines 48 hours after administration of various doses of a peptide of SEQ ID NO: 15 conjugated to MMAE by a Val-Cit-PAB linker (SEQ ID NO: 15-B peptide conjugate).

FIG. 15 shows HPLC traces, 3D models, and sequences of SEQ ID NO: 15 peptide, SEQ ID NO: 1 peptide, and SEQ ID NO: 2 peptide.

FIG. 15A shows HPLC traces of a peptide of SEQ ID NO: 15 where solid traces show protein reduced with dithioreitol (DTT) and dashed traces show non-reduced proteins. Below each HPLC trace is the corresponding model of a peptide of SEQ ID NO: 15, where dark gray regions indicate regions of positive charge, medium-colored grey regions indicate regions of negative charge, and light gray regions indicate regions of neutral charge.

FIG. 15B shows HPLC traces of a peptide of SEQ ID NO: 1 where solid traces show protein reduced with dithioreitol (DTT) and dashed traces show non-reduced proteins. Below each HPLC trace is the corresponding model of a peptide of SEQ ID NO: 1, where dark gray regions indicate regions of positive charge, medium-colored gray regions indicate regions of negative charge, and light gray regions indicate regions of neutral charge.

FIG. 15C shows HPLC traces of a peptide of SEQ ID NO: 2 where solid traces show protein reduced with dithioreitol (DTT) and dashed traces show non-reduced proteins. Below each HPLC trace is the corresponding model of a peptide of SEQ ID NO: 2, where dark gray regions indicate regions of positive charge, medium-colored gray regions indicate regions of negative charge, and light gray regions indicate regions of neutral charge

FIG. 15D shows sequences of peptides of SEQ ID NO: 15, SEQ ID NO: 1, and SEQ ID NO: 2.

FIG. 16 illustrates a schematic of a method of manufacturing of a peptide of the disclosure.

FIG. 17 shows liquid scintillation counting and quantification of the concentration of a radiolabeled peptide of SEQ ID NO: 15 recovered in plasma at several time points after administration of 2 μCi/33 nmol of radiolabeled peptides in female Harlan athymic nude mice via intravenous (IV) administration shown in circle data points, intraperitoneal (IP) administration shown in square data points, oral (PO) administration shown in triangle data points, and subcutaneous (SC) administration shown in inverted triangle data points. Each data point shows mean and error bars indicating standard deviation (n=3).

FIG. 18 shows analysis and quantification of signal in plasma by tandem HPLC and liquid scintillation of a radiolabeled peptide of SEQ ID NO: 15 (SEQ ID NO: 15-r) at several time points after administration of 2 μCi/33 nmol of radiolabeled peptides in female Harlan athymic nude mice via different routes. HPLC was used to separate peptide fragments and liquid scintillation counting was used to quantify the radioactive signal of intact peptides or peptide fragment.

FIG. 18A shows the signal in plasma after intravenous (IV) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 18B shows the signal in plasma after intraperitoneal (IP) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 18C shows the signal in plasma after oral (PO) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 18D shows the signal in plasma after subcutaneous (SC) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 19 shows liquid scintillation counting and quantification of the concentration of a radiolabeled peptide of SEQ ID NO: 15 (SEQ ID NO: 15-r) in urine at several time points after administration of 2 μCi/33 nmol of SEQ ID NO: 15-r in female Harlan athymic nude mice via intravenous (IV) administration, intraperitoneal (IP) administration, oral (PO) administration, and subcutaneous (SC) administration. Each data point shows mean and error bars indicating standard deviation (n=3).

FIG. 20 shows analysis and quantification of signal in urine by tandem HPLC and liquid scintillation of a radiolabeled peptide of SEQ ID NO: 15 (SEQ ID NO: 15-r) at several time points after administration of 2 μCi/33 nmol of SEQ ID NO: 15-r in female Harlan athymic nude mice via different routes. HPLC was used to separate peptide fragments and liquid scintillation counting was used to quantify the radioactive signal of intact peptides or peptide fragment.

FIG. 20A shows the signal in urine after intravenous (IV) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 20B shows the signal in urine after intraperitoneal (IP) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 20C shows the signal in urine after oral (PO) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 20D shows the signal in urine after subcutaneous (SC) administration of a radiolabeled peptide of SEQ ID NO: 15 (SEQ ID NO: 15-r). SEQ ID NO: 15-r was spiked in as a positive control for intact peptide and Glycine was spiked in as a negative control for metabolized peptide.

FIG. 21 shows quantification of signal in tissues after administration of a panel of peptides conjugated to AF647 to mice. Peptide-fluorophore conjugates were administered intravenously to mice bearing RH28 flank tumors at 10 nmol per mouse. Mice were euthanized four hours post-administration, organs were necropsied, and tissues were analyzed ex vivo using an IVIS imager. All tested sequences are homologs. P-values were determined using an unpaired Student's t-test.

FIG. 21A shows average radiant efficiency fluorescence from peptide-fluorophore conjugates in RH28 flank tumors.

FIG. 21B shows the tumor to liver ratio of average radiant efficiency fluorescence from peptide-fluorophore conjugates.

FIG. 22 shows quantification of signal in tissues after administration of a peptide of SEQ ID NO: 15 conjugated to AlexaFluor647 (SEQ ID NO: 15-A) and a peptide of SEQ ID NO: 36 (the D-amino acid version of SEQ ID NO: 15) conjugated to AlexaFluor647 (SEQ ID NO: 36-A), and free AlexaFluor647 (AF647) to mice. Peptide-fluorophore conjugates were administered intravenously to mice bearing RH28 flank tumors at 10 nmol per mouse. Mice were euthanized one hour post-administration, organs were necropsied, and tissues were analyzed ex vivo using an IVIS imager. P-values were determined using an unpaired Student's t-test.

FIG. 22A shows average radiant efficiency fluorescence from a peptide of SEQ ID NO: 15 conjugated to AlexaFluor647 (SEQ ID NO: 15-A) and a peptide of SEQ ID NO: 36 (the D-amino acid version of SEQ ID NO: 15) conjugated to AlexaFluor647 (SEQ ID NO: 36-A), and free AlexaFluor647 (AF647) (as a negative control) in RH28 flank tumors.

FIG. 22B shows average radiant efficiency fluorescence from a peptide of SEQ ID NO: 15 conjugated to AlexaFluor647 (SEQ ID NO: 15-A) and a peptide of SEQ ID NO: 36 (the D-amino acid version of SEQ ID NO: 15) conjugated to AlexaFluor647 (SEQ ID NO: 36-A), and free AlexaFluor647 (AF647) (as a negative control) in livers.

FIG. 22C shows average radiant efficiency fluorescence from a peptide of SEQ ID NO: 15 conjugated to AlexaFluor647 (SEQ ID NO: 15-A) and a peptide of SEQ ID NO: 36 (the D-amino acid version of SEQ ID NO: 15) conjugated to AlexaFluor647 (SEQ ID NO: 36-A), and free AlexaFluor647 (AF647) (as a negative control) in kidneys.

FIG. 22D shows fluorescence images of radiant efficiency in necropsied tumor, liver, and kidney taken using an IVIS imager, which correspond to the bar graphs in FIG. 22A, FIG. 22B, and FIG. 22C. A representative tissue is shown from one mouse in each group—a peptide of SEQ ID NO: 15 conjugated to AlexaFluor647 (SEQ ID NO: 15-A) and a peptide of SEQ ID NO: 36 (the D-amino acid version of SEQ ID NO: 15) conjugated to AlexaFluor647 (SEQ ID NO: 36-A), and free AlexaFluor647 (AF647).

FIG. 23 shows quantification of signal in tissues after administration of a peptide of SEQ ID NO: 15 conjugated to AlexaFluor647 (SEQ ID NO: 15-A), SEQ ID NO: 15-A with a 50-fold excess of unlabeled SEQ ID NO: 13, and free AlexaFluor647 (AF647). P-values were determined using an unpaired Student's t-test.

FIG. 23A shows average radiant efficiency fluorescence from a peptide of SEQ ID NO: 15 conjugated to AlexaFluor647 (SEQ ID NO: 15-A), SEQ ID NO: 15-A with a 50-fold excess of unlabeled SEQ ID NO: 13, and free AlexaFluor647 (AF647) (as a negative control) in RH28 flank tumors. Alignment of sequences of SEQ ID NO: 15 and SEQ ID NO: 13 is also shown (* denotes a conserved residue).

FIG. 23B shows average radiant efficiency fluorescence from a peptide of SEQ ID NO: 15 conjugated to AlexaFluor647 (SEQ ID NO: 15-A), SEQ ID NO: 15-A with a 50-fold excess of unlabeled SEQ ID NO: 13, and free AlexaFluor647 (AF647) (as a negative control) in livers.

FIG. 23C shows average radiant efficiency fluorescence from a peptide of SEQ ID NO: 15 conjugated to AlexaFluor647 (SEQ ID NO: 15-A), SEQ ID NO: 15-A with a 50-fold excess of unlabeled SEQ ID NO: 13, and free AlexaFluor647 (AF647) (as a negative control) in kidneys.

FIG. 23D shows fluorescence images of radiant efficiency in necropsied tumor, liver, and kidney taken using an IVIS imager, which correspond to the bar graphs in FIG. 23A, FIG. 23B, and FIG. 23C. A representative tissue is shown from one mouse in each group—administration of peptide of SEQ ID NO: 15 conjugated to AlexaFluor647 (SEQ ID NO: 15-A), SEQ ID NO: 15-A with a 50-fold excess of unlabeled SEQ ID NO: 13, or free AlexaFluor647 (AF647).

FIG. 24 shows cell viability curves after treatment with monomethyl auristatin E (MMAE), MMAE with a Val-Cit-PAB linker (linker-MMAE), and MMAE conjugated to a peptide of SEQ ID NO: 15 via a Val-Cit-PAB linker (SEQ ID NO: 15-B). A673 Ewing's sarcoma cells were treated for two days without or with cathepsins, an enzyme that cleaves the linker, and with increasing concentrations of MMAE, linker-MMAE, or SEQ ID NO: 15-B. Cell viability was assessed using a Cell Titer Glo assay.

FIG. 24A shows cell viability curves for A673 cells incubated with MMAE, Val-Cit-PAB linker-MMAE (linker-MMAE), or MMAE conjugated to a peptide of SEQ ID NO: 15 via a Val-Cit-PAB linker (SEQ ID NO: 15-B).

FIG. 24B shows cell viability curves for A673 cells incubated with cathepsins, MMAE and cathepsin, Val-Cit-PAB linker-MMAE (linker-MMAE) and cathepsin, or MMAE conjugated to a peptide of SEQ ID NO: 15 via a Val-Cit-PAB linker (SEQ ID NO: 15-B) and cathepsin.

FIG. 25 shows that small molecules can modulate the efficacy of a tumor targeting peptide-dye conjugate of this disclosure. The Approved Oncology Drugs Plated Set VII was obtained from NCI and included histone deacetylase (HDAC) inhibitors such as vorinostat, belinostat, panobinostat, and pentostatin. The tested set also included tyrosine kinase inhibitors, protease inhibitors, and anthracyclines. A375 cells and RH28 cells were plated at 10,000 cells per well in 96 well plates and allowed to adhere overnight.

FIG. 25A shows the median fluorescence for each drug tested in A375 cells. Drugs from the 129 compound set were added at 10 μM and allowed to incubate for 16 hours. A peptide of SEQ ID NO: 15 conjugated to AlexaFluor647 (SEQ ID NO: 15-A) was added at 1 μM for four hours. Cells were washed three times with PBS-FBS and once in PBS. Cells were trypsinized, resuspended in PBS-FBS-DAPI, and assessed for average fluorescence as compared to untreated cells by flow cytometry analysis. Each data point represents a drug from Approved Oncology Drugs Plated Set VII incubated with SEQ ID NO: 15-A. Median fluorescence above 1 indicates that the efficacy was enhanced and median fluorescence below 1 indicates that the efficacy was reduced.

FIG. 25B shows the median fluorescence for each drug tested in RH28 cells. Drugs from the 129 compound set were added at 10 μM and allowed to incubate for 16 hours. A peptide of SEQ ID NO: 15 conjugated to AlexaFluor647 (SEQ ID NO: 15-A) was added at 1 μM for four hours. Cells were washed three times with PBS-FBS and once in PBS. Cells were trypsinized, resuspended in PBS-FBS-DAPI, and assessed for average fluorescence as compared to untreated cells by flow cytometry analysis. Each data point represents a drug from the Approved Oncology Drugs Plated Set VII incubated with SEQ ID NO: 15-A. Median fluorescence above 1 indicates that the efficacy was enhanced and median fluorescence below 1 indicates that the efficacy was reduced.

FIG. 25C shows the median fluorescence in A375 cells for five drugs from the Approved Oncology Drugs Plated Set VII including BEZ235, bleomycin, cytarabine, palbociclib, and vorinostat. Each drug was administered at increasing concentrations with 1 μM of peptide of SEQ ID NO: 15 conjugated to AlexaFluor647 (SEQ ID NO: 15-A) and the median fluorescence was plotted. Median fluorescence above 1 indicates that the efficacy was enhanced and median fluorescence below 1 indicates that the efficacy was reduced.

FIG. 25D shows the median fluorescence in RH28 cells for five drugs from the Approved Oncology Drugs Plated Set VII including BEZ235, bleomycin, cytarabine, palbociclib, and vorinostat. Drug were administered at increasing concentrations with 1 μM of peptide of SEQ ID NO: 15 conjugated to AlexaFluor647 (SEQ ID NO: 15-A) and the median fluorescence was plotted. Median fluorescence above 1 indicates that the efficacy was enhanced and median fluorescence below 1 indicates that the efficacy was reduced.

FIG. 26 shows structural analysis of SEQ ID NO: 10 peptide, SEQ ID NO: 11 peptide, SEQ ID NO: 12 peptide, SEQ ID NO: 14 peptide, and SEQ ID NO: 26 peptide.

FIG. 26A shows a cartoon representation of structural alignment of SEQ ID NO: 10 peptide, SEQ ID NO: 11 peptide, SEQ ID NO: 12 peptide, SEQ ID NO: 14 peptide, and SEQ ID NO: 26 peptide with a pacifastin structural fold with a 180° view along the X axis. Ovals indicate molecular surface involved in chymotrypsin binding and inhibition.

FIG. 26B shows a surface representation of SEQ ID NO: 10 peptide, SEQ ID NO: 11 peptide, SEQ ID NO: 12 peptide, SEQ ID NO: 14 peptide, and SEQ ID NO: 26 peptide with chymotrypsin binding site represented by medium-colored gray and dark gray shades with medium-colored gray shades indicating conserved sequences.

FIG. 26C shows a general sequence motif and logo for a peptide that can bind chymotrypsin with chymotrypsin binding sites indicated by arrows (speckled or unfilled) and conserved residues indicated by unfilled arrows (N=8).

FIG. 26D shows a sequence motif for a peptide that exhibits tumor homing propensity (N=35).

FIG. 26E shows a cartoon representation of SEQ ID NO: 10 peptide, SEQ ID NO: 11 peptide, SEQ ID NO: 12 peptide, SEQ ID NO: 14 peptide, and SEQ ID NO: 26 peptide.

FIG. 26F shows an electrostatic surface representation of the cartoon representations of SEQ ID NO: 10 peptide, SEQ ID NO: 11 peptide, SEQ ID NO: 12 peptide, SEQ ID NO: 14 peptide, and SEQ ID NO: 26 peptide as shown in FIG. 26E at 180° along the Y axis.

### DETAILED DESCRIPTION

The present disclosure relates to compositions and methods for treatment of tumors or cancerous cells. In some embodiments, the compositions and methods herein utilize a peptide that homes, migrates to, distributes to, accumulates in, is directed to, and/or binds to cancerous cells following administration to a subject. In some embodiments, the homing peptides of the present disclosure are used to deliver an active agent to a tissue or cell thereof. The active agent can exert a therapeutic effect on the targeted tissue or cell thereof. For example, in certain embodiments, the peptide allows for localized delivery of a cytotoxic drug to a cancerous tissue or cell thereof. In certain embodiments, the homing peptides of the present disclosure are used to image the targeted tissue or cell. For example, the peptide allows for localized delivery of a fluorophore dye, enabling imaging and visualization of a cancerous tissue or cell.

Many types of tumors can be difficult to treat. Often, the prognosis of the patient is directly influenced by the ability of drug therapies to effectively kill the cancerous cells and on the precision with which the cancer cells can be surgically resected. For example, one challenge in treating tumors is that many drug treatments are systemic, and therefore, the efficacy of their use is constrained by the toxicity of systemic use. Another challenge is that the current methods of intra-operative imaging of cancerous tissues can fail to precisely depict tumor margins or small foci of cancerous cells. Instead, resection can be dependent upon the surgeon's ability to visually recognize tumor or physically locate it by touch in a surgical setting, which can be an imprecise method to identify the tumor margins or foci.

The present disclosure describes a class of peptides derived from knottins that can home, distribute to, target, be directed to, accumulate in, migrate to, and/or bind to cancerous or diseased cells, and be used as carriers of active drugs, peptides, or molecules to treat the cancerous or diseased cells. A peptide that homes, distributes to, targets, migrates to, accumulates in, and/or binds one or more specific cancerous or diseased regions, tissues, structures or cells can have fewer off-target and potentially negative effects. The present disclosure also describes a class of peptides that can be used as carriers of detectable agents, such as dyes, metals, or radioisotopes, to visualize cancerous or diseased tissues and cells thereof. As described herein, an active agent or a detectable agent can be linked to a peptide of the disclosure.

The peptides described herein that selectively home, distribute to, migrate to, accumulate in, and/or bind to cancerous or diseased tissues can increase the efficacy of new or existing drugs by targeting them to the cancerous tissues of interest. Targeting drugs to the cancerous tissues of interests can improve the therapeutic index of new or known drugs, and can limit off-target distribution of drugs to other organs such as the liver.

The present disclosure also provides a new class of peptides that can act as carriers to deliver an active agent or a detectable agent to sarcoma, and can be used for either or both therapeutic and imaging purposes. Sarcomas are malignant tumors that can develop from bone or soft tissue. Ewing's sarcoma is a type of sarcoma that develops in bone, and mainly impacts young adolescents. Soft tissue sarcomas form in the arms and legs and can grow large before becoming symptomatic as a result of their location in soft tissues. If detected early, sarcomas generally can have favorable outcomes unless the disease becomes metastatic and spreads to other regions of the body. As described herein, an active agent or a detectable agent can be linked to a peptide of the disclosure and the linked peptide conjugates, peptide-active agent or peptide-detectable agent, can selectively home, distribute to, migrate to, accumulate in, and/or bind to sarcomas.

The present disclosure also provides a new class of peptides that can act as carriers to deliver an active agent or a detectable agent to Burkitt's lymphoma. Burkitt's lymphoma are cancers that primarily affect B lymphocytes and are caused by Epstein-Barr virus. As described herein, an active agent or a detectable agent can be linked to a peptide of the disclosure and the linked peptide conjugates, peptide-active agent or peptide-detectable agent, can selectively home, distribute to, migrate to, accumulate in, and/or bind to sarcomas.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

Peptides

Knottins are a class of peptides, usually ranging from about 11 to about 81 amino acids in length that can be folded into a compact structure. Knottins can be assembled into a complex tertiary structure that is characterized by a number of intramolecular disulfide crosslinks and may contain beta strands and other secondary structures. The presence of the disulfide bonds can give knottins remarkable environmental stability, allowing them to withstand extremes of temperature and pH and to resist the proteolytic enzymes of the blood stream. The rigidity of knottins also can allow them to bind to targets without paying the “entropic penalty” that a floppy peptide accrues upon binding a target. For example, binding can be adversely affected by the loss of entropy that occurs when a peptide binds a target to form a complex. Therefore, “entropic penalty” can be the adverse effect on binding, and the greater the entropic loss that occurs upon this binding, the greater the “entropic penalty.” Furthermore, unbound molecules that are flexible can lose more entropy when forming a complex than molecules that are rigidly structured, because of the loss of flexibility when bound up in a complex. However, rigidity in the unbound molecule also can increase specificity by limiting the number of complexes that molecule can form. The knotted peptides can bind targets with antibody-like affinity. A wider examination of the sequence structure and sequence identity or homology of knottins reveals that they have arisen by convergent evolution in all kinds of animals and plants. In animals, they are typically found in venoms, for example, the venoms of spiders and scorpions and have been implicated in the modulation of ion channels. The knottin proteins of plants can inhibit the proteolytic enzymes of animals or have antimicrobial activity, suggesting that knottins can function in the defense of plants.

The present disclosure provides peptides that can comprise or be derived from these knotted peptides (or knottins). As used herein, the term “knotted peptide” is considered to be interchangeable with the terms “knottin” and “optide.”

The peptides of the present disclosure can comprise cysteine amino acid residues. In some cases, the peptide has at least 6 cysteine amino acid residues. In some cases, the peptide has at least 8 cysteine amino acid residues. In other cases, the peptide has at least 10 cysteine amino acid residues, at least 12 cysteine amino acid residues, at least 14 cysteine amino acid residues, or at least 16 cysteine amino acid residues.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the disulfide bridges form a knot. A disulfide bridge can be formed between cysteine residues, for example, between cysteines 1 and 4, 2 and 5, and 3 and 6. In some cases, one disulfide bridge passes through a loop formed by the other two disulfide bridges, for example, to form the knot. In other cases, the disulfide bridges can be formed between any two cysteine residues.

In certain embodiments, knotted peptides are assembled into a complex tertiary structure that is characterized by a number of intramolecular disulfide crosslinks, and optionally contain beta strands and other secondary structures such as an alpha helix. For example, knotted peptides include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures include peptide having two parallel helices linked by two disulfide bridges without β-sheets (e.g., hefutoxin).

A knotted peptide can comprise at least one amino acid residue in an L configuration. A knotted peptide can comprise at least one amino acid residue in a D configuration. In some embodiments, a knotted peptide is 15-40 amino acid residues long. In other embodiments, a knotted peptide is 11-57 amino acid residues long. In still other embodiments, a knotted peptide is 11-81 amino acid residues long. In further embodiments, a knotted peptide is at least 20 amino acid residues long.

In certain embodiments, these kinds of peptides can be derived from a knottin protein called chymotrypsin inhibitor (CTI), which is also known as Locusta migratoria chymotrypsin inhibitor II (LCMI-II) and Pars intercerebralis major peptide C (PMP-C). These 36 amino acid knotted peptides can be potent protease inhibitors and cationic. The peptides of this disclosure can be derived from the knottin CTI. A peptide of this disclosure can be a serine protease inhibitor. A peptide of this disclosure can serine protease inhibitor, which can be a pacifastin family member. A peptide of this disclosure can be a Locusta migratoria chymotrypsin inhibitor II (LCMI-II). A peptide of this disclosure can be a variant of a serine protease inhibitor, pacifastin family member, and/or a Locusta migratoria chymotrypsin inhibitor II (LCMI-II). A peptide can be a non-naturally occurring peptide. Non-naturally occurring can refer to an article not caused by or existing in nature in its natural form. TABLE 1 shows exemplary peptides of this disclosure. Upper case letters indicate L-amino acids and lower case letters indicate D-amino acids.

TABLE 1
Exemplary Peptides

| SEQ ID NO | Amino Acid Sequence |
|---|---|
| SEQ ID NO: 1 | GSSCEPGRTFEDECNTCRCGADGRSAACTLEACPNQ |
| SEQ ID NO: 2 | GSSCEPGRTFADACNTCRCGADGRSAACTLAACPNQ |
| SEQ ID NO: 3 | GSSCEPGKTFRDRCNTCRCGADGRSAACTLRACPNQ |
| SEQ ID NO: 4 | GSSCEPGRTFKDRCNTCRCGADGRSAACTLRACPNQ |
| SEQ ID NO: 5 | GSSCEPGRTFRDKCNTCRCGADGRSAACTLRACPNQ |
| SEQ ID NO: 6 | GSSCEPGRTFRDRCNTCRCGADGKSAACTLRACPNQ |
| SEQ ID NO: 7 | GSSCEPGRTFRDRCNTCRCGADGRSAACTLKACPNQ |
| SEQ ID NO: 8 | GSSCEPGTTFRDRCNTCRCGSDGRSAACTLRACPQ |
| SEQ ID NO: 9 | GSSCTPGTTFRDRCNTCRCSSNGRSAACTLRACPP |
| | GSY |
| SEQ ID NO: 10 | GSSCTPGTTFRNRCNTCRCGSNGRSASCTLMACPP |
| | GSY |
| SEQ ID NO: 11 | GSSCTPGATFRNRCNTCRCGSNGRSASCTLMACPP |
| | GSY |
| SEQ ID NO: 12 | GSSCQPGTTYQRGCNTCRCLEDGQTEACTLRLC |
| SEQ ID NO: 13 | GSSCTPGATYREGCNICRCRSDGRSGACTRRICPV |
| | DSN |
| SEQ ID NO: 14 | GSSCQPGTTFRRDCNTCVCNRDGTNAACTLRACL |
| SEQ ID NO: 15 | GSSCEPGRTFRDRCNTCRCGADGRSAACTLRACPNQ |
| SEQ ID NO: 16 | GSSCRPGRTFRDRCNTCRCGADGRSAACTLRACPNQ |
| SEQ ID NO: 17 | GSSCEPGETFRDRCNTCRCGADGRSAACTLRACPNQ |
| SEQ ID NO: 18 | GSSCEPGRTFEDRCNTCRCGADGRSAACTLRACPNQ |
| SEQ ID NO: 19 | GSSCEPGRTFRRRCNTCRCGADGRSAACTLRACPNQ |
| SEQ ID NO: 20 | GSSCEPGRTFRDECNTCRCGADGRSAACTLRACPNQ |
| SEQ ID NO: 21 | GSSCEPGRTFRDRCDTCRCGADGRSAACTLRACPNQ |
| SEQ ID NO: 22 | GSSCEPGRTFRDRCNTCECGADGRSAACTLRACPNQ |
| SEQ ID NO: 23 | GSSCEPGRTFRDRCNTCRCGARGRSAACTLRACPNQ |
| SEQ ID NO: 24 | GSSCEPGRTFRDRCNTCRCGADGESAACTLRACPNQ |
| SEQ ID NO: 25 | GSSCEPGRTFRDRCNTCRCGADGRSAACTLEACPNQ |
| SEQ ID NO: 26 | GSSCEPGRTFRDRCNTCKCGADGRSAACTLRACPNQ |
| SEQ ID NO: 27 | GSSRRRRRRRRCEPGRTFRDRCNTCRCGADGRSAAC |
| | TLRAC |
| SEQ ID NO: 28 | GSSCLPNETFRFDCNSCRCNDDGRTAACTLMLC |
| SEQ ID NO: 29 | GSEISCEPGKTFKDKCNTCRCGADGKSAACTLKACP |
| | NQ |
| SEQ ID NO: 30 | GSEISCEPGKTFRDRCNTCRCGADGRSAACTLRACP |
| | NQ |
| SEQ ID NO: 31 | GSEISCEPGRTFKDRCNTCRCGADGRSAACTLRACP |
| | NQ |
| SEQ ID NO: 32 | GSEISCEPGRTFRDKCNTCRCGADGRSAACTLRACP |
| | NQ |
| SEQ ID NO: 33 | GSEISCEPGRTFRDRCNTCRCGADGKSAACTLRACP |
| | NQ |
| SEQ ID NO: 34 | GSEISCEPGRTFRDRCNTCRCGADGRSAACTLKACP |
| | NQ |
| SEQ ID NO: 35 | GSEISCEPGKTFKDKCNTCRCGADGKSAACTLKACP |
| | NQ |
| SEQ ID NO: 37 | SCEPGRTFEDECNTCRCGADGRSAACTLEACPNQ |
| SEQ ID NO: 38 | SCEPGRTFADACNTCRCGADGRSAACTLAACPNQ |
| SEQ ID NO: 39 | SCEPGKTFRDRCNTCRCGADGRSAACTLRACPNQ |
| SEQ ID NO: 40 | SCEPGRTFKDRCNTCRCGADGRSAACTLRACPNQ |
| SEQ ID NO: 41 | SCEPGRTFRDKCNTCRCGADGRSAACTLRACPNQ |
| SEQ ID NO: 42 | SCEPGRTFRDRCNTCRCGADGKSAACTLRACPNQ |
| SEQ ID NO: 43 | SCEPGRTFRDRCNTCRCGADGRSAACTLKACPNQ |
| SEQ ID NO: 44 | SCEPGTTFRDRCNTCRCGSDGRSAACTLRACPQ |
| SEQ ID NO: 45 | SCTPGTTFRDRCNTCRCSSNGRSAACTLRACPPGSY |
| SEQ ID NO: 46 | SCTPGTTFRNRCNTCRCGSNGRSASCTLMACPPGSY |
| SEQ ID NO: 47 | SCTPGATFRNRCNTCRCGSNGRSASCTLMACPPGSY |
| SEQ ID NO: 48 | SCQPGTTYQRGCNTCRCLEDGQTEACTLRLC |
| SEQ ID NO: 49 | SCTPGATYREGCNICRCRSDGRSGACTRRICPVDSN |
| SEQ ID NO: 50 | SCQPGTTFRRDCNTCVCNRDGTNAACTLRACL |
| SEQ ID NO: 51 | SCEPGRTFRDRCNTCRCGADGRSAACTLRACPNQ |
| SEQ ID NO: 52 | SCRPGRTFRDRCNTCRCGADGRSAACTLRACPNQ |
| SEQ ID NO: 53 | SCEPGETFRDRCNTCRCGADGRSAACTLRACPNQ |
| SEQ ID NO: 54 | SCEPGRTFEDRCNTCRCGADGRSAACTLRACPNQ |
| SEQ ID NO: 55 | SCEPGRTFRRRCNTCRCGADGRSAACTLRACPNQ |
| SEQ ID NO: 56 | SCEPGRTFRDECNTCRCGADGRSAACTLRACPNQ |
| SEQ ID NO: 57 | SCEPGRTFRDRCDTCRCGADGRSAACTLRACPNQ |
| SEQ ID NO: 58 | SCEPGRTFRDRCNTCECGADGRSAACTLRACPNQ |
| SEQ ID NO: 59 | SCEPGRTFRDRCNTCRCGARGRSAACTLRACPNQ |
| SEQ ID NO: 60 | SCEPGRTFRDRCNTCRCGADGESAACTLRACPNQ |
| SEQ ID NO: 61 | SCEPGRTFRDRCNTCRCGADGRSAACTLEACPNQ |
| SEQ ID NO: 62 | SCEPGRTFRDRCNTCKCGADGRSAACTLRACPNQ |
| SEQ ID NO: 63 | SRRRRRRRRCEPGRTFRDRCNTCRCGADGRSAACTL |
| | RAC |
| SEQ ID NO: 64 | SCLPNETFRFDCNSCRCNDDGRTAACTLMLC |
| SEQ ID NO: 65 | EISCEPGKTFKDKCNTCRCGADGKSAACTLKACPNQ |
| SEQ ID NO: 66 | EISCEPGKTFRDRCNTCRCGADGRSAACTLRACPNQ |
| SEQ ID NO: 67 | EISCEPGRTFKDRCNTCRCGADGRSAACTLRACPNQ |
| SEQ ID NO: 68 | EISCEPGRTFRDKCNTCRCGADGRSAACTLRACPNQ |
| SEQ ID NO: 69 | EISCEPGRTFRDRCNTCRCGADGKSAACTLRACPNQ |
| SEQ ID NO: 70 | EISCEPGRTFRDRCNTCRCGADGRSAACTLKACPNQ |

In some embodiments, a peptide of the present disclosure can include a D-amino acid version of any one of SEQ ID NO: 1-SEQ ID NO: 35 or SEQ ID NO: 37-SEQ ID NO: 71. For example, a peptide of this disclosure can include a peptide with the sequence: gsscepgrtfrdrcntcrcgadgrsaactlracpnq (SEQ ID NO: 36), which is the D-amino acid version of SEQ ID NO: 15. In such sequences, D-amino acids are represented by lower case letters. As another example, a peptide of this disclosure can include a peptide with the sequence: scepgrtfrdrcntcrcgadgrsaactlracpnq (SEQ ID NO: 72), which is the D-amino acid version of SEQ ID NO: 51. SEQ ID NO: 72 is also a non-GS version of SEQ ID NO: 36.

In some embodiments, the peptides of the present disclosure comprise a sequence having cysteine residues at one or more of positions 4, 12, 14, 17, 19, 22, 25, 27, 28, 33, 36, and 41. In other embodiments, the peptides of the present disclosure comprise a sequence having cysteine residues at one or more positions 6, 16, 19, 21, 30, and 35. For example, in certain embodiments, a peptide comprises a sequence having a cysteine residue at position 4. For example, in certain embodiments, a peptide comprises a sequence having a cysteine residue at position 6. In certain embodiments, a peptide comprises a sequence having a cysteine residue at position 12. In certain embodiments, a peptide comprises a sequence having a cysteine residue at position 14. For example, in certain embodiments, a peptide comprises a sequence having a cysteine residue at position 16. In certain embodiments, a peptide comprises a sequence having a cysteine residue at position 17. In certain embodiments, a peptide comprises a sequence having a cysteine residue at position 19. For example, in certain embodiments, a peptide comprises a sequence having a cysteine residue at position 21. In certain embodiments, a peptide comprises a sequence having a cysteine residue at position 22. In certain embodiments, a peptide comprises a sequence having a cysteine residue at position 25. In certain embodiments, a peptide comprises a sequence having a cysteine residue at position 27. In certain embodiments, a peptide comprises a sequence having a cysteine residue at position 28. For example, in certain embodiments, a peptide comprises a sequence having a cysteine residue at position 30. In certain embodiments, a peptide comprises a sequence having a cysteine residue at position 33. For example, in certain embodiments, a peptide comprises a sequence having a cysteine residue at position 35. In certain embodiments, a peptide comprises a sequence having a cysteine residue at position 36. In certain embodiments, a peptide comprises a sequence having a cysteine residue at position 41.

In some embodiments, the first cysteine residue in the sequence is disulfide bonded with the 4th cysteine residue in the sequence, the 2^{nd} cysteine residue in the sequence is disulfide bonded to the 5^{th} cysteine residue in the sequence, and the 3^{rd} cysteine residue in the sequence is disulfide bonded to the 6^{th} cysteine residue in the sequence. In some embodiments, the 1^{st} cysteine residue in the sequence is disulfide bonded to the 4^{th} cysteine residue in the sequence, the second cysteine residue in the sequence is disulfide bonded to the 6^{th} cysteine residue in the sequence, the 3^{rd} cysteine residue in the sequence is disulfide bonded to the 7^{th} cysteine residue in the sequence, and the 5^{th} cysteine residue in the sequence is disulfide bonded to the 8^{th} cysteine residue in the sequence.

In some instances, the peptide can contain only one lysine residue, or no lysine residues. In some instances, some or all of the lysine residues in the peptide are replaced with arginine residues. In some instances, some or all of the methionine residues in the peptide are replaced by leucine or isoleucine. In some instances, some or all of the tryptophan residues in the peptide are replaced by phenylalanine or tyrosine. In some instances, some or all of the asparagine residues in the peptide are replaced by glutamine. In some cases, the N-terminus of the peptide is blocked, such as by an acetyl group. Alternatively or in combination, in some instances, the C-terminus of the peptide is blocked, such as by an amide group.

In some cases, the first two N-terminal amino acids shown (GS) in SEQ ID NO: 1-SEQ ID NO: 36, or such N-terminal amino acids (GS) can be absent or substituted by any other one or two amino acids.

Generally, the NMR solution structures of related structural homologs can be used to inform mutational strategies that may improve the folding, stability, manufacturability, while maintaining a particular biological function. They can be used to predict the 3D pharmacophore of a group of structurally homologous scaffolds, as wells as to predict possible graft regions of related proteins to create chimeras with improved properties. The general strategy for producing homologs can include identification of a charged surface patch of a protein, mutation of critical amino acid positions and loops, and testing of sequences. This strategy can be used to design peptides with improved properties or to correct deleterious mutations that complicate folding and manufacturability. For example, a peptide of SEQ ID NO: 1 and a peptide of SEQ ID NO: 2 were manufactured and found to be unable to fold. Accordingly, three amino acid positions (R11, R13, and R31) were chosen to be individually mutated to improve stability, yielding peptides of SEQ ID NO: 18, SEQ ID NO: 20, and SEQ ID NO: 25. Improved folding was observed in SEQ ID NO: 18, SEQ ID NO: 20, and SEQ ID NO: 25 as compared to SEQ ID NO: 1 and SEQ ID NO: 2.

In some instances, the peptide is any one of SEQ ID NO: 1-SEQ ID NO: 35 or a functional fragment thereof. In other embodiments, the peptide of the disclosure further comprises a peptide with 99%, 95%, 90%, 85%, or 80% sequence identity or homology to any one of SEQ ID NO: 1-SEQ ID NO: 35 or fragment thereof. In some instances, the peptide is any one of SEQ ID NO: 37-SEQ ID NO: 71 or a functional fragment thereof. In other embodiments, the peptide of the disclosure further comprises a peptide with 99%, 95%, 90%, 85%, or 80% sequence identity or homology to any one of SEQ ID NO: 37-SEQ ID NO: 71 or fragment thereof.

In other instances, the peptide can be a peptide that is homologous to any one of SEQ ID NO: 1-SEQ ID NO: 35 or a functional fragment thereof. The term “homologous” is used herein to denote peptides having at least 70%, at least 80%, at least 90%, at least 95%, or greater than 95% sequence identity or homology to a sequence of any one of SEQ ID NO: 1-SEQ ID NO: 35, SEQ ID NO: 37-SEQ ID NO: 71, or a functional fragment thereof.

In still other instances, the variant nucleic acid molecules of a peptide of any one of SEQ ID NO: 1-SEQ ID NO: 35 or SEQ ID NO: 37-SEQ ID NO: 71 can be identified by either a determination of the sequence identity or homology of the encoded peptide amino acid sequence with the amino acid sequence of any one of SEQ ID NO: 1-SEQ ID NO: 35 or SEQ ID NO: 37-SEQ ID NO: 71, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 35 or SEQ ID NO: 37-SEQ ID NO: 71 (or its complement) under stringent washing conditions, in which the wash stringency is equivalent to 0.5×−2×SSC with 0.1% SDS at 55-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 35 or SEQ ID NO: 37-SEQ ID NO: 71. Alternatively, peptide variants of any one of SEQ ID NO: 1-SEQ ID NO: 35 or SEQ ID NO: 37-SEQ ID NO: 71 can be characterized as nucleic acid molecules (1) that remain hybridized with a nucleic acid molecule having the nucleotide sequence of any one of SEQ ID NO: 1-SEQ ID NO: 35 or SEQ ID NO: 37-SEQ ID NO: 71 (or its complement) under highly stringent washing conditions, in which the wash stringency is equivalent to 0.1×−0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 35 or SEQ ID NO: 37-SEQ ID NO: 71.

Percent sequence identity or homology is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, determination of structure can typically be accompanied by evaluating the activity of modified molecules.

In further embodiments, the peptide fragment comprises a contiguous fragment of any one of SEQ ID NO: 1-SEQ ID NO: 35 or SEQ ID NO: 37-SEQ ID NO: 71 that is at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, wherein the peptide fragment is selected from any portion of the peptide.

The peptides of the present disclosure can further comprise positively charged amino acid residues. In some cases, the peptide has at least 1 positively charged residue. In some cases, the peptide has at least 2 positively charged residues. In some cases, the peptide has at least 3 positively charged residues. In other cases, the peptide has at least 4 positively charged residues, at least 5 positively charged residues, at least 6 positively charged residues, at least 7 positively charged residues, at least 8 positively charged residues or at least 9 positively charged residues. While the positively charged residues can be selected from any positively charged amino acid residues, in some embodiments, the positively charged residues are either K, or R, or a combination of K and R.

The peptides of the present disclosure can further comprise neutral amino acid residues. In some cases, the peptide has 35 or fewer neutral amino acid residues. In other cases, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some cases the peptide has 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any neutral charged amino acid residues, in some embodiments, the negative amino acid residues are either E, or D, or a combination of both E and D.

At physiological pH, peptides can have a net charge, for example, of −5, −4, −3, −2, −1, 0, +1, +2, +3, +4, or +5. When the net charge is zero, the peptide can be uncharged or zwitterionic. In some embodiments, the peptide contains one or more disulfide bonds and has a positive net charge at physiological pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10. In some cases, the engineering of one or more mutations within a peptide yields a peptide with an altered isoelectric point, charge, surface charge, or rheology at physiological pH. Such engineering of a mutation to a peptide derived from a CTI can change the net charge of the complex, for example, by decreasing the net charge by 1, 2, 3, 4, or 5, or by increasing the net charge by 1, 2, 3, 4, or 5. Suitable amino acid modifications for improving the rheology and potency of a peptide can include conservative or non-conservative mutations. A peptide can comprises at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to a CTI sequence that the peptide is derived from. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to a CTI sequence that the peptide is derived from. In some embodiments, mutations can be engineered within a peptide to provide a peptide that has a desired charge or stability at physiological pH.

The present disclosure can also encompass fucosylated CTI, CTI variants, or any one SEQ ID NO: 1-SEQ ID NO: 26 or SEQ ID NO: 28-SEQ ID NO: 35 or SEQ ID NO: 37-SEQ ID NO: 62 or SEQ ID NO: 64-SEQ ID NO: 71. Fucosylation is a type of glycosylation, and can be implicated in cancers. Glycosylation can be a form of post-translational modification of peptides and proteins, where residues can be modified with glycans. Fucosylation can involve the attachment of a fucose ring to amino acid residues and can be mediated by fucosyltransferases. In some embodiments, a peptide of the disclosure can be fucosylated at the first threonine amino acid residue, Thr-9. Fucosylation at Thr-9 can be stabilized by Thr-16 and Arg-18. Fucosylation can impact the pharmacokinetics, solubility, or immunogenicity of a given peptide or protein. In certain embodiments, CTI and CTI variants that comprise any one of SEQ ID NO: 1-SEQ ID NO: 26 or SEQ ID NO: 28-SEQ ID NO: 35 can be fucosylated. In further embodiments, CTI and the CTI variants that comprise any one of SEQ ID NO: 1-SEQ ID NO: 26 or SEQ ID NO: 28-SEQ ID NO: 35 can be fucosylated at the first threonine amino acid residue. In still further embodiments, CTI and the CTI variants that comprise any one of SEQ ID NO: 1-SEQ ID NO: 26 or SEQ ID NO: 28-SEQ ID NO: 35 can be fucosylated at Thr-9. In some embodiments, a peptide of the disclosure can be fucosylated at the first threonine amino acid residue, Thr-7. Fucosylation at Thr-7 can be stabilized by Thr-14 and Arg-16. Fucosylation can impact the pharmacokinetics, solubility, or immunogenicity of a given peptide or protein. In certain embodiments, CTI and CTI variants that comprise any one of SEQ ID NO: 37-SEQ ID NO: SEQ ID NO: 62 or SEQ ID NO: 64-SEQ ID NO: 71 can be fucosylated. In further embodiments, CTI and the CTI variants that comprise any one of SEQ ID NO: 37-SEQ ID NO: 62 or SEQ ID NO: 64-SEQ ID NO: 71 can be fucosylated at the first threonine amino acid residue. In still further embodiments, CTI and the CTI variants that comprise any one of SEQ ID NO: 37-SEQ ID NO: 62 or SEQ ID NO: 64-SEQ ID NO: 71 can be fucosylated at Thr-7.

The present disclosure also encompasses multimers of the various peptides described herein. Examples of multimers include dimers, trimers, tetramers, pentamers, hexamers, heptamers, and so on. A multimer may be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The present disclosure further includes peptide scaffolds that, e.g., can be used as a starting point for generating additional peptides. In some embodiments, these scaffolds can be derived from a variety of knotted peptides or knottins. A suitable peptide for scaffolds can include, but is not limited to, chymotrypsin inhibitor II (LCMI-II), which can also be referred to as PMP-C.

In some cases the peptide comprises the sequence of any one of SEQ ID NO: 1-SEQ ID NO: 35 or SEQ ID NO: 37-SEQ ID NO: 71. In some embodiments, the peptide sequence is flanked by additional amino acids. One or more additional amino acids can, for example, confer a desired in vivo charge, isoelectric point, chemical conjugation site, stability, or physiologic property to a peptide.

Two or more peptides can share a degree of sequence identity or homology and share similar properties in vivo. For instance, a peptide can share a degree of sequence identity or homology with any one of the peptides of SEQ ID NO: 1-SEQ ID NO: 35 or SEQ ID NO: 37-SEQ ID NO: 71. In some cases, one or more peptides of the disclosure can have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. Various methods and software programs can be used to determine the homology between two or more peptides, such as NCBI BLAST, Clustal W, MAFFT, Clustal Omega, AlignMe, Praline, or another suitable method or algorithm.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

Chemical Modifications

A peptide can be chemically modified one or more of a variety of ways. In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation may be accomplished through the use of reductive methylation with formaldehyde and sodium cyanoborohydride.

A chemical modification can, for instance, extend the half-life of a peptide or change the biodistribution or pharmacokinetic profile. A chemical modification can comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region can be a fusion Fc-peptide. A polyamino acid can include, for example, a poly amino acid sequence with repeated single amino acids (e.g., poly glycine), and a poly amino acid sequence with mixed poly amino acid sequences (e.g., gly-ala-gly-ala (SEQ ID NO: 84)) that may or may not follow a pattern, or any combination of the foregoing.

In some embodiments, the peptides of the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide of the present disclosure. In other embodiments, the peptide of the present disclosure can include post-translational modifications (e.g., methylation and/or amidation), which can affect, e.g., serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof. In other embodiments, the peptides of the present disclosure are coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin.

In some embodiments, the first two N-terminal amino acids (GS) of SEQ ID NO: 1-SEQ ID NO: 35 serve as a spacer or linker in order to facilitate conjugation or fusion to another molecule, as well as to facilitate cleavage of the peptide from such conjugated or fused molecules. In some embodiments, the fusion proteins or peptides of the present disclosure can be conjugated to other moieties that, e.g., can modify or effect changes to the properties of the peptides.

Active Agent Peptide Conjugates

Peptides according to the present disclosure can be conjugated or fused to an agent for use in the treatment of tumors and cancers. For example, in certain embodiments, the peptides described herein are fused to another molecule, such as an active agent that provides a functional capability. A peptide can be fused with an active agent through expression of a vector containing the sequence of the peptide with the sequence of the active agent. In various embodiments, the sequence of the peptide and the sequence of the active agent are expressed from the same Open Reading Frame (ORF). In various embodiments, the sequence of the peptide and the sequence of the active agent can comprise a contiguous sequence. The peptide and the active agent can each retain similar functional capabilities in the fusion peptide compared with their functional capabilities when expressed separately. In certain embodiments, examples of active agents include other peptides.

Furthermore, for example, in certain embodiments, the peptides described herein are attached to another molecule, such as an active agent that provides a functional capability. In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015)). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, an antibody, a single chain variable fragment (scFv, or single chain Fv), an antibody fragment, an aptamer, a cytokine, an interferon, an interleukin, a hormone, an enzyme, a growth factor, a checkpoint inhibitor, a PD-1 inhibitor, a PD-L1 inhibitor, a CTLA4 inhibitor, a CD antigen, a chemokine, a neurotransmitter, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic molecule, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc domain or an Fc region, or an active fragment or a modification thereof. In some embodiments, the peptide is covalently or non-covalently liked to an immunomodulatory agent, a T cell activating agent, a macrophage activating agent, a natural killer cell activating agent, or an agent modulates proteins that provide stimulatory or inhibitory signals to the immune system. In some embodiments, the peptide is covalently or non-covalently linked to an active agent, e.g., directly or via a linker. For example, cytotoxic molecules that can be used include auristatins, MMAE, MMAF, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, and BACE inhibitors. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). Exemplary linkers suitable for use with the embodiments herein are discussed in further detail below.

As compared to antibody-drug conjugates (e.g., Adcetris, Kadcyla, Mylotarg), in some aspects the peptide conjugated to an active agent as described herein may exhibit better penetration of solid tumors due to its smaller size. In certain aspects, the peptide conjugated to an active agent as described herein may be able to carry different or higher doses of active agents as compared to antibody-drug conjugates. In still other aspects, the peptide conjugated to an active agent as described herein may have better site specific delivery of defined drug ratio as compared to antibody-drug conjugates. In other aspects, the peptide may be amenable to solvation in organic solvents (in addition to water), which may allow more synthetic routes for solvation and conjugation of a drug (which often has low aqueous solubility) and higher conjugation yields, higher ratios of drug conjugated to peptide (versus an antibody), and/or reduce aggregate/high molecular weight species formation during conjugation. Additionally, a unique amino acid residue(s) may be introduced into the peptide via a residue that is not otherwise present in the short sequence or via inclusion of a non-natural amino acid, allowing site specific conjugation to the peptide. Changing the formulation of a cytotoxic agent can increase the therapeutic window of the agent, can increase the amount of agent delivered to a tumor, or can improve pharmacokinetics, pharmacodynamics, or efficacy of an agent, which can be demonstrated by binding paclitaxel to albumin in Abraxane (Desai, N., Clin Cancer Res., 12(12): 3869 (2006)). Likewise, linking a cytoxic agent to any peptide of this disclosure can increase the effective tumor delivery of the agent at acceptable tolerability to a patient.

The peptides or fusion peptides of the present disclosure can also be conjugated to other moieties that can serve other roles, such as providing an affinity handle (e.g., biotin) for retrieval of the peptides from tissues or fluids. For example, peptides or fusion peptides of the present disclosure can also be conjugated to biotin. In addition to extension of half-life, biotin could also act as an affinity handle for retrieval of peptides or fusion peptides from tissues or other locations. In some embodiments, fluorescent biotin conjugates that can act both as a detectable label and an affinity handle can be used. Non-limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates could include chemiluminescent compounds, colloidal metals, luminescent compounds, enzymes, radioisotopes, and paramagnetic labels. In some embodiments, the peptide described herein can also be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

Additionally, more than one peptide sequence derived from CTI knottin protein can be present on or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

### Detectable Agent Peptide Conjugates

A peptide can be conjugated to an agent used in imaging, research, therapeutics, theranostics, pharmaceuticals, chemotherapy, chelation therapy, targeted drug delivery, and radiotherapy. In some embodiments, a peptide is conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, or 10 detectable agents can be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Other embodiments of the present disclosure provide peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is fused with, or covalently or non-covalently linked to the agent, e.g., directly or via a linker. Exemplary linkers suitable for use with the embodiments herein are discussed in further detail below.

### Linkers

Peptides according to the present disclosure that home, migrate to, distribute to, accumulate in, are directed to, and/or bind cancerous or diseased cells can be attached to another moiety (e.g., an active agent or an detectable agent), such as a small molecule, a second peptide, a protein, an antibody, an antibody fragment, a single chain Fv, an aptamer, polypeptide, polynucleotide, a fluorophore, a radioisotope, a radionuclide chelator, a polymer, a biopolymer, a fatty acid, an acyl adduct, a chemical linker, or sugar or other active agent or detectable agent described herein through a linker, or directly in the absence of a linker. In the absence of a linker, for example, an active agent or an detectable agent can be fused to the N-terminus or the C-terminus of a peptide to create an active agent or detectable agent fusion peptide. In other embodiments, the link can be made by a peptidic fusion via reductive alkylation.

Direct attachment is possible by covalent attachment of a peptide to a region of the other molecule. For example, an active agent or a detectable agent can be fused to the N-terminus or the C-terminus of a peptide to create an active agent or detectable agent fusion peptide. As another example, the peptide can be attached at the N-terminus, an internal lysine residue, or the C-terminus to a terminus of the amino acid sequence of the other molecule by a linker. If the attachment is at an internal lysine residue, the other molecule can be linked to the peptide at the epsilon amine of the internal lysine residue. In some further examples, the peptide can be attached to the other molecule by a side chain, such as the side chain of a lysine, serine, threonine, cysteine, tyrosine, aspartic acid, a non-natural amino acid residue, or glutamic acid residue. A linker can be an amide bond, an ester bond, an ether bond, a carbamate bond, a carbonate bond, a carbon-nitrogen bond, a triazole, a macrocycle, an oxime bond, a hydrazone bond, a carbon-carbon single, double, or triple bond, a disulfide bond, a two carbon bridge between two cysteines, a three carbon bridge between two cysteines, or a thioether bond. In still other embodiments, the peptide comprises a non-natural amino acid, wherein the non-natural amino acid is an insertion, appendage, or substitution for another amino acid, and the peptide is linked to the active agent at the non-natural amino acid by a linker. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single, double, or triple bond, a disulfide bond, a thioether bond, or other linker as described herein) may be used to link other molecules.

Attachment via a linker involves incorporation of a linker moiety between the other molecule and the peptide. The peptide and the other molecule can both be covalently attached to the linker. The linker can be cleavable, non-cleavable, self-immolating, hydrophilic, or hydrophobic. The linker has at least two functional groups, one bonded to the other molecule, and one bonded to the peptide, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment include functional groups capable of forming, for example, an amide bond, an ester bond, an ether bond, a carbonate bond, a carbamate bond, a carbon-nitrogen bond, a triazole, a macrocycle, an oxime bond, a hydrazone bond, a carbon-carbon single, double, or triple bond, a disulfide bond or a thioether bond. Non-limiting examples of functional groups capable of forming such bonds include amino groups; carboxyl groups; aldehyde groups; azide groups; alkyne and alkene groups; ketones; hydrazides; hydrazines; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; maleimides; linkers containing maleimide groups that are designed to hydrolyze; maleimidocaproyl; MCC ([N-maleimidomethyl]cyclohexane-1-carboxylate); N-ethylmaleimide; maleimide alkane; mc-vc-PABC; DUBA (DuocarmycinhydroxyBenzamide-Azaindole linker); SMCC Succinimidyl-4-(N-maleimidomethyl) cyclohexane-1-carboxylate; SPDP (N-succinimidyl-3-(2-pyridyldithio) propionate); SPDB N-succinimidyl-4-(2-pyridyldithio) butanoate; sulfo-SPDB N-succinimidyl-4-(2-pyridyldithio)-2-sulfo butanoate; SPP N-succinimidyl 4-(2-pyridyldithio)pentanoate; a dithiopyridylmaleimide (DTM); a hydroxylamine, a vinyl-halo group; haloacetamido groups; bromoacetamido; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion include alkylene, alkenylene, alkynylene, polyether, such as polyethylene glycol (PEG), oligoethylene glycol, polyester, polyamide, polyamino acids, polypeptides, cleavable peptides, Val-Cit, Phe-Lys, Val-Lys, Val-Ala, other peptide linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link molecules together include click chemistry, copper-free click chemistry, HIPS ligation, Staudinger ligation, and hydrazine-iso-Pictet-Spengler.

Non-limiting examples of linkers include:

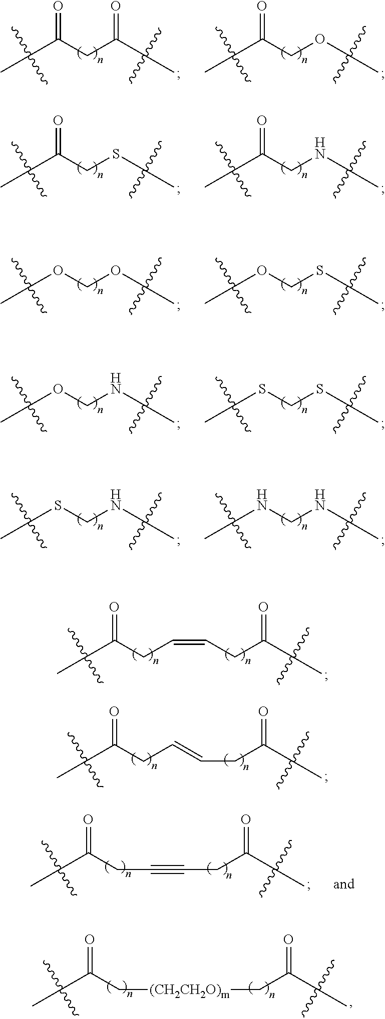

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, the linker can release the active agent in an unmodified form. In other embodiments, the active agent can be released with chemical modification. In still other embodiments, catabolism can release the active agent still linked to parts of the linker and/or peptide.

In some embodiments, the use of a cleavable linker can permit release of the conjugated moiety (e.g., a therapeutic agent) from the peptide, e.g., after targeting to the tumor or cancerous cell. In other embodiments, the use of a cleavable linker can permit the release of the conjugated therapeutic from the peptide after penetrating a tumor, binding a cancer cell, or being internalized be a cancer cell. In some cases the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, cathepsins, peptidases, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters is faster compared to the hydrolysis of linkers with bulky groups next to an ester carbonyl. As an additional example, the rate of disulfide cleavage or exchange with unhindered disulfides is faster compared to the rate of disulfide cleavage or exchange of linkers with bulky groups near disulfide bonds. Protease sites can also affect cleavage rates. A bulky group can be a methyl group, an ethyl group, a phenyl group, a ring, or an isopropyl group, or any group that provides steric bulk. In some cases, the steric bulk can be provided by the drug itself, such as by ketorolac when conjugated via its carboxylic acid. The rate of hydrolysis of the linker can be tuned according to the residency time of the conjugate in the target location. For example, when a peptide is cleared from a tumor, or the brain, relatively quickly, the linker can be tuned to rapidly hydrolyze. When a peptide has a longer residence time in the target location, a slower hydrolysis rate can allow for extended delivery of an active agent. “Programmed hydrolysis in designing paclitaxel prodrug for nanocarrier assembly” Sci Rep 2015, 5, 12-23 Fu et al., provides an example of modified hydrolysis rates.

### Crystal Structure

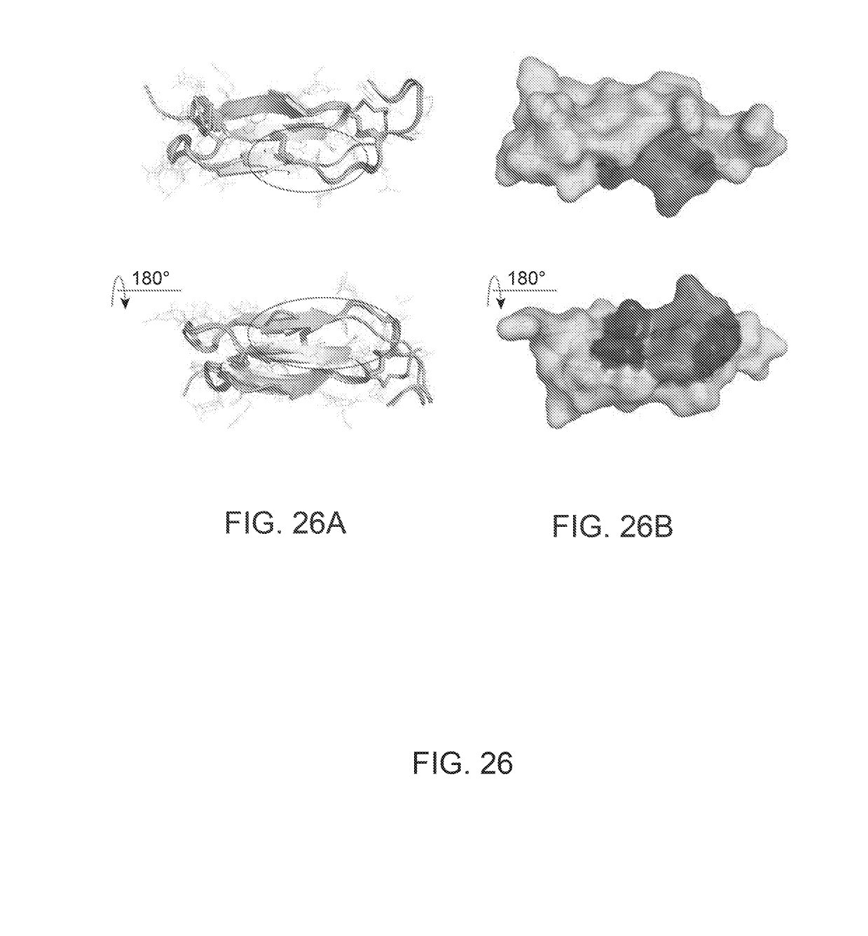

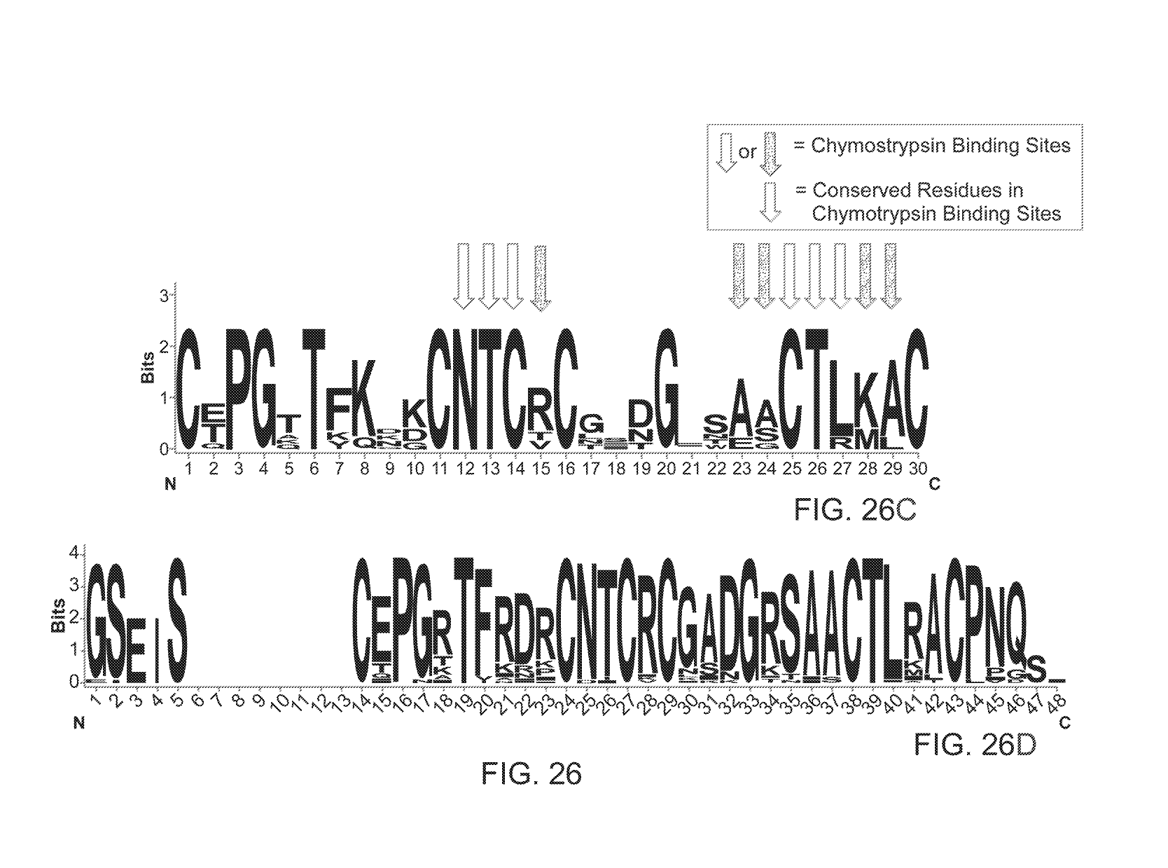

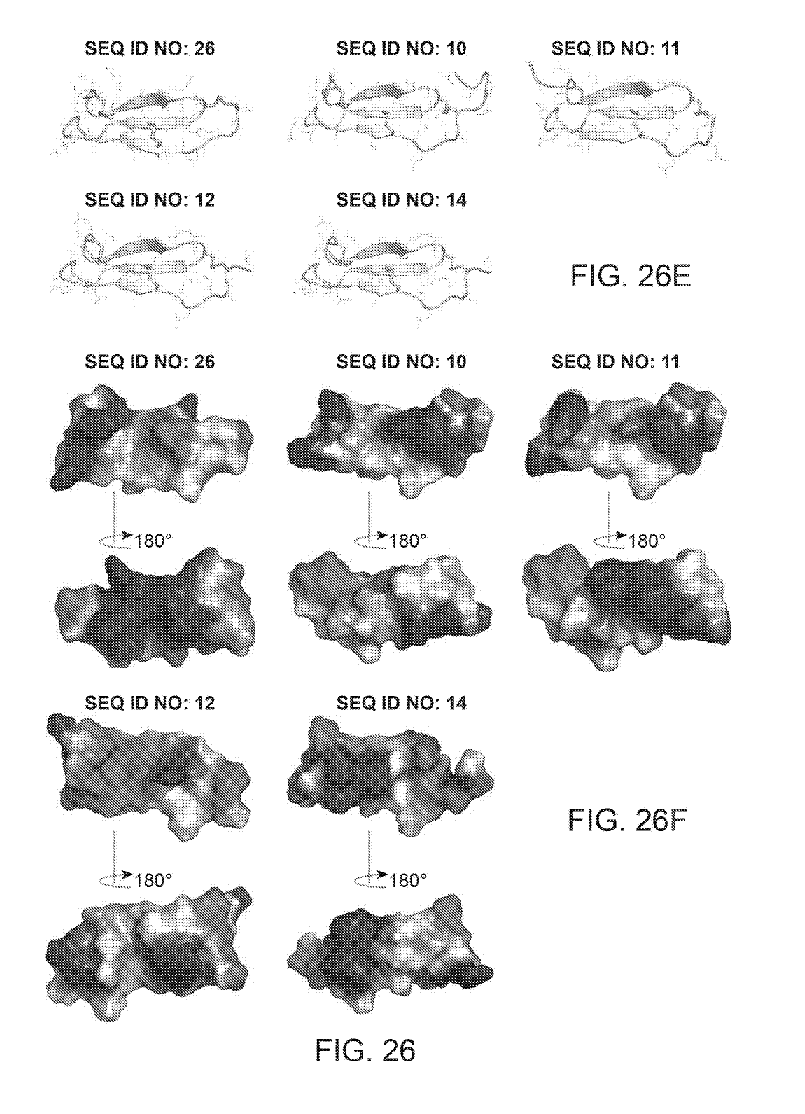

In some embodiments, the crystal structure of any peptide of this disclosure can be solved in order to spatially map each atom in a given peptide. Solving the crystal structure of the peptide can yield information on the spatial orientation, positioning, and interaction of amino acids. Thus, in some embodiments, the crystal structure of a peptide can provide information on conserved structural elements that can play a role in tumor homing and binding function. The crystal structure can also be used to provide guidance on how sequence elements can play a role in folding and determining the structure of the peptide. For example, solving the crystal structure can indicate how conserved amino acids play a role in determining the three dimensional structure of the peptide overall and determining functional elements. For example, the solved crystal structures of peptides, such as peptides of SEQ ID NO: 10-SEQ ID NO: 12 and SEQ ID NO: 14, shows the chymotrypsin binding sites, which can be used to determine a consensus residue for peptides with both a chymotrypsin binding site and the ability to home to a tumor. The consensus residue can be determined by sequence alignment of SEQ ID NO: 10 peptide, SEQ ID NO: 11 peptide, SEQ ID NO: 12 peptide, SEQ ID NO: 14 peptide, and SEQ ID NO: 26 peptide. FIG. 26C shows a general sequence motif and logo for a peptide that can bind chymotrypsin with chymotrypsin binding sites indicated by arrows (speckled or unfilled) and conserved residues indicated by unfilled arrows (N=8), in which a peptide of this disclosure can comprise a sequence with the amino acid residues of the chymotrypsin binding sites. The general sequence shown in this figure is a pacifastin sequence motif that was generated using the structures with the following sequences: SEQ ID NO: 10 peptide, SEQ ID NO: 11 peptide, SEQ ID NO: 12 peptide, SEQ ID NO: 14 peptide, and SEQ ID NO: 26 peptide as well as the structures of 1 kgm (SEQ ID NO: 81: EVTCEPGTTFKDKCNTCRCGSDGKSAACTLKACPQ), 1 pcm (SEQ ID NO: 82: EISCEPGKTFKDKCNTCRCGADGKSAACTLKACPNQ) and 1wo9 (SEQ ID NO: 83: AGECTPGQTKKQDCNTCTCTPTGIW-GCTRKACRTT), which can be downloaded from the protein databank. The structures of 1 kgm, 1 pcm, and 1wo9 can be used as reference structures, and were used here as reference structures in generating the motif of FIG. 26C. FIG. 26D shows a sequence motif for a peptide that exhibits tumor homing propensity (N=35), in which a peptide of this disclosure can comprise a sequence with the conserved amino acid residues. All peptides of SEQ ID NO: 1-SEQ ID NO: 35 were used to generate the motif shown in FIG. 26D.

Based on this analysis, a peptide that homes, migrates to, distributes to, accumulates in, is directed to, and/or binds to cancerous cells can comprise a member of a family with a sequence of GSSCXPGXTXXXXCNTCXCXXDGXXXXCTLXXCXXXXX (SEQ ID NO: 73), wherein X can independently be any number of any amino acid or no amino acid. In some embodiments, the peptide can comprise a sequence of GSSCXPGXTXXXXCNTCXCXXDGXXXXCTLXXCXXXXX (SEQ ID NO: 74), wherein the following residues may be independently interchanged in this sequence: M, I, L, and V; G and A; S and T; Q and N; and X can independently be any number of any amino acid or no amino acid. In some embodiments, the peptide can comprise a sequence of GSSCX¹PGX²TX³X⁴X⁵X⁶CNTCX⁷CX⁸X⁹DGX¹⁰X¹¹X¹²X¹³CTLX¹⁴X¹⁵CX¹⁶X¹⁷X¹⁸X¹⁹X²⁰ (SEQ ID NO: 75), wherein X¹ can be selected from E and T, X² can be selected from R, T, and A, X³ can be selected from F and Y, X⁴ can be selected from R and Q, X⁵ can be selected from D, R, and N, X⁶ can be selected from G, R, and D, X⁷ can be selected from R, V, and K, X⁸ can be selected from L, G, and N, X⁹ can be selected from E, S, R, and A, X¹⁰ can be selected from Q, R, and T, X¹¹ can be selected from T, S, and N, X¹² can be selected from E and A, X¹³ can be selected from A and S, X¹⁴ can be selected from R and M, X¹⁵ can be selected from L and A, X¹⁶ can be selected from P, L, and S, or can be absent, X¹⁷ can be selected from P, and S, or can be absent, X¹⁸ can be G or can be absent, X¹⁹ can be S or can be absent, and X²⁰ can be Y or can be absent. In some embodiments, the peptide can comprise a sequence of GSSCX¹PGX²TX³X⁴X⁵X⁶CNTCX⁷CX⁸X⁹DGX¹⁰X¹¹X¹²X¹³CMX¹⁴X¹⁵CX¹⁶X¹⁷X¹⁸X¹⁹X²⁰ (SEQ ID NO: 76), wherein X¹ can be selected from E and T, X² can be selected from R, T, and A, X³ can be selected from F and Y, X⁴ can be selected from R and Q, X⁵ can be selected from D, R, and N, X⁶ can be selected from G, R, and D, X⁷ can be selected from R, V, and K, X⁸ can be selected from L, G, and N, X⁹ can be selected from E, S, R, and A, X¹⁰ can be selected from Q, R, and T, X¹¹ can be selected from T, S, and N, X¹² can be selected from E and A, X¹³ can be selected from A and S, X¹⁴ can be selected from R and M, X¹⁵ can be selected from L and A, X¹⁶ can be selected from P, L, and S, or can be absent, X¹⁷ can be selected from P, and S, or can be absent, X¹⁸ can be G or can be absent, X¹⁹ can be S or can be absent, and X²⁰ can be Y or can be absent, and wherein the following residues can be independently interchanged in this sequence: K and R; M, I, L, and V; G and A; S and T; and Q and N. In some embodiments, the peptide can comprise a sequence of SCXPGXTXXXXCNTCXCXXDGXXXXCTLXXCXXXXX (SEQ ID NO: 77), wherein X can independently be any number of any amino acid or no amino acid. In some embodiments, the peptide can comprise a sequence of SCXPGXTXXXXCNTCXCXXDGXXXXCTLXXCXXXXX (SEQ ID NO: 78), wherein the following residues may be independently interchanged in this sequence: M, I, L, and V; G and A; S and T; Q and N; and X can independently be any number of any amino acid or no amino acid. In some embodiments, a peptide can comprise a sequence of GSSCX¹PGX²TX³X⁴X⁵X⁶CNTCX⁷CX⁸X⁹DGX¹⁰X¹¹X¹²X¹³CMX¹⁴X¹⁵CX¹⁶X¹⁷X¹⁸X¹⁹X²⁰ (SEQ ID NO: 79), wherein X¹ can be selected from E and T, X² can be selected from R, T, and A, X³ can be selected from F and Y, X⁴ can be selected from R and Q, X⁵ can be selected from D, R, and N, X⁶ can be selected from G, R, and D, X⁷ can be selected from R, V, and K, X⁸ can be selected from L, G, and N, X⁹ can be selected from E, S, R, and A, X¹⁰ can be selected from Q, R, and T, X¹¹ can be selected from T, S, and N, X¹² can be selected from E and A, X¹³ can be selected from A and S, X¹⁴ can be selected from R and M, X¹⁵ can be selected from L and A, X¹⁶ can be selected from P, L, and S, or can be absent, X¹⁷ can be selected from P, and S, or can be absent, X¹⁸ can be G or can be absent, X¹⁹ can be S or can be absent, and X²⁰ can be Y or can be absent. In some embodiments, the peptide can comprise a sequence of GSSCX¹PGX²TX³X⁴X⁵X⁶CNTCX⁷CX⁸X⁹DGX¹⁰X¹¹X¹²X¹³CMX¹⁴X¹⁵CX¹⁶X¹⁷X¹⁸X¹⁹X²⁰ (SEQ ID NO: 80), wherein X¹ can be selected from E and T, X² can be selected from R, T, and A, X³ can be selected from F and Y, X⁴ can be selected from R and Q, X⁵ can be selected from D, R, and N, X⁶ can be selected from G, R, and D, X⁷ can be selected from R, V, and K, X⁸ can be selected from L, G, and N, X⁹ can be selected from E, S, R, and A, X¹⁰ can be selected from Q, R, and T, X¹¹ can be selected from T, S, and N, X¹² can be selected from E and A, X¹³ can be selected from A and S, X¹⁴ can be selected from R and M, X¹⁵ can be selected from L and A, X¹⁶ can be selected from P, L, and S, or can be absent, X¹⁷ can be selected from P, and S, or can be absent, X¹⁸ can be G or can be absent, X¹⁹ can be S or can be absent, and X²⁰ can be Y or can be absent, and wherein the following residues can be independently interchanged in this sequence: K and R; M, I, L, and V; G and A; S and T; and Q and N.

### Peptide Stability

A peptide of the present disclosure can be stable in various biological conditions. For example, any peptide of SEQ ID NO: 1-SEQ ID NO: 35 or SEQ ID NO: 37-SEQ ID NO: 70 can exhibit resistance to reducing agents, proteases, oxidative conditions, or acidic conditions.

In some cases, biologic molecules (such as peptides and proteins) can provide therapeutic functions, but such therapeutic functions are decreased or impeded by instability caused by the in vivo environment (Moroz et al., Adv Drug Deliv Rev, 101:108-21 (2016), Mitragotri et al., Nat Rev Drug Discov, 13(9):655-72 (2014), Bruno et al., Ther Deliv, (11):1443-67 (2013), Sinha et al., Crit Rev Ther Drug Carrier Syst., 24(1):63-92 (2007), Hamman et al., BioDrugs, 19(3):165-77 (2005)). For instance, the GI tract can contain a region of low pH (e.g., pH ˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they may be unable to provide a therapeutic function on intracellular targets. In addition, tumors can be protease-rich environments, thus resistance to proteases can improve the ability of a peptide or peptide-active agent conjugate to have therapeutic antitumor activity. Therefore, peptides that are resistant to reducing agents, proteases, and low pH may be able to provide enhanced therapeutic effects or enhance the therapeutic efficacy of co-formulated or conjugated active agents in vivo.

Additionally, oral delivery of drugs can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function.

Peptide Resistance to Reducing Agents.

In some embodiments, a peptide of the present disclosure can be reduction resistant. Peptides of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body and in cells, and can reduce disulfide bonds. As another example, a peptide can become reduced during trafficking of a peptide across the gastrointestinal epithelium after oral administration. A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to loss of functionality due to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001 M-0.0001 M, 0.0001 M-0.001 M, 0.001 M-0.01 M, 0.01 M-0.05 M, 0.05 M-0.1 M, for 15 minutes or more. In some embodiments, the reducing agent used to determine peptide stability can be dithiothreitol (DTT), Tris(2-carboxyethyl)phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent.

Peptide Resistance to Proteases.

The stability of peptides of this disclosure can be determined by resistance to degradation by proteases. In some embodiments, a peptide of this disclosure can be resistant to protease degradation. Proteases, also referred to as peptidases or proteinases, are enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, esterases, serum proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, peptides of this disclosure can resist degradation by any class of protease. In certain embodiments, peptides of this disclosure resist degradation by pepsin (which can be found in the stomach), trypsin (which can be found in the duodenum), serum proteases, or any combination thereof. In some embodiments, the proteases used to determine peptide stability can be pepsin, trypsin, chymotrypsin, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, and elafin), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a protease.

Peptide Stability in Acidic Conditions.

Peptides of this disclosure can be administered in biological environments that are acidic. For example, after oral administration, peptides can experience acidic environmental conditions in the gastric fluids of the stomach and gastrointestinal (GI) tract. The pH of the stomach can range from ˜1-4 and the pH of the GI tract ranges from acidic to normal physiological pH descending from the upper GI tract to the colon. In addition, the vagina, late endosomes, and lysosomes can also have acidic pH values, such as less than pH 7. These acidic conditions can lead to denaturation of peptides and proteins into unfolded states. Unfolding of peptides and proteins can lead to increased susceptibility to subsequent digestion by other enzymes as well as loss of biological activity of the peptide. In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to simulated gastric fluid. In some embodiments, low pH solutions such as simulated gastric fluid or citrate buffers can be used to determine peptide stability.

Peptide Stability at High Temperatures.

In some embodiments, a peptide of the present disclosure can be resistant to elevated temperature. Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for at least 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

### Methods of Manufacture

Various expression vector/host systems can be utilized for the production of the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system using a variety of known techniques employed in protein and peptide synthesis.

In some cases, a host cell produces a peptide that has an attachment point for a drug. An attachment point could comprise a lysine residue, an N-terminus, a cysteine residue, a cysteine disulfide bond, or a non-natural amino acid. The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

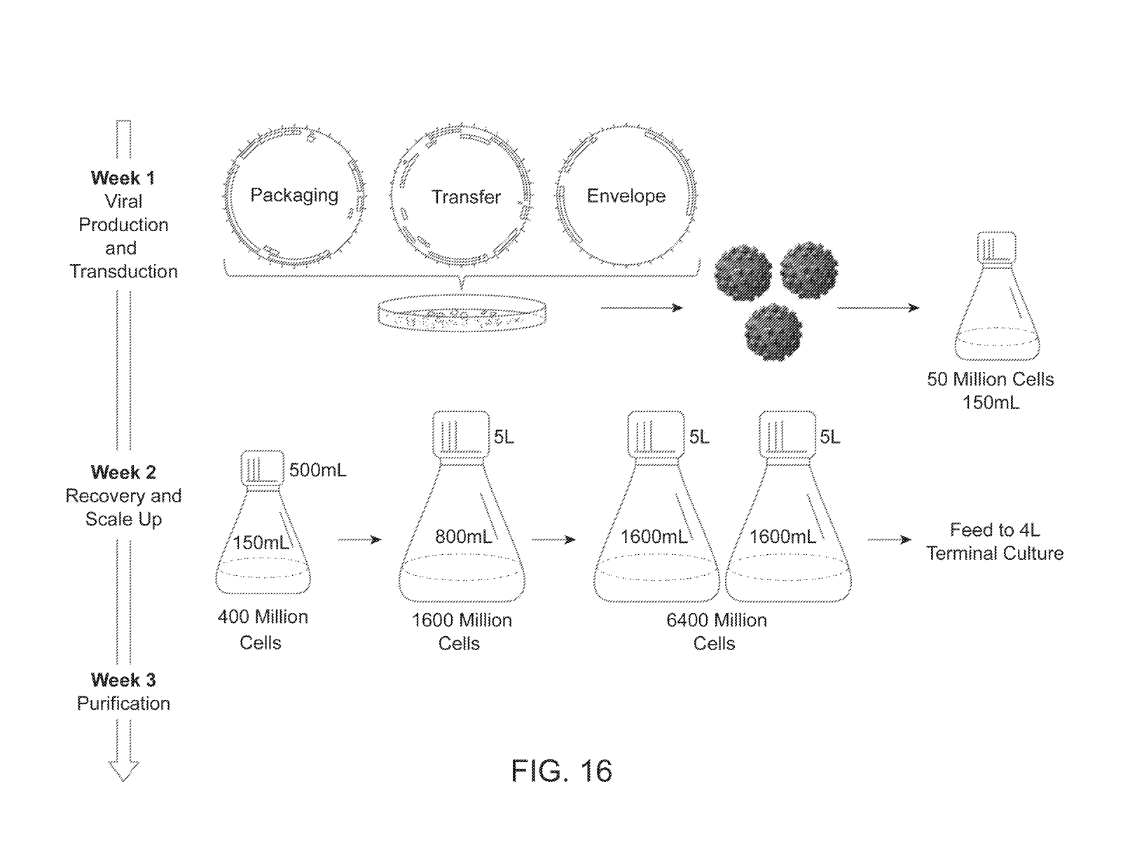

FIG. 16 illustrates a schematic of a method of manufacturing a construct that expresses a peptide of the disclosure or any one of SEQ ID NO: 1-SEQ ID NO: 35 peptides provided herein.

In other aspects, the peptides of the present disclosure can be prepared by conventional solid phase chemical synthesis techniques, for example according to the Fmoc solid phase peptide synthesis method (Fmoc solid phase peptide synthesis, a practical approach, edited by W. C. Chan and P. D. White, Oxford University Press, 2000) or by conventional solution phase peptide synthesis. Refolding and disulfide bond formation can be executed by methods known in the art, such as incubation of the peptide at a mildly basic pH in the presence of a redox pair such as reduced and oxidized cysteine or reduced and oxidized by glutathione or by air, either after cleavage and protecting group removal and purification, or while still on the resin. Peptide fragments can also be made synthetically or recombinantly and the joined together.

### Pharmaceutical Compositions of Peptides

A pharmaceutical composition of the disclosure can be a combination of any peptide described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, and topical administration. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously. A peptide described herein can be administered to a subject and cross the blood brain barrier of a subject.

A peptide of the disclosure can be applied directly to an organ, or an organ tissue or cells, such as cancer cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), each of which is incorporated by reference in its entirety.

### Pharmacokinetics of Peptides

The pharmacokinetics of any of the peptides of this disclosure can be determined after administration of the peptide via different routes of delivery. For example, the pharmacokinetic parameters of a peptide of this disclosure can be quantified intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, or topical administration. Peptides of the present disclosure can be analyzed by using tracking agents such as radiolabels or fluorophores. For example, a radiolabeled peptide of this disclosure can be administered via various routes of administration. Peptide concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein can relate to pharmacokinetics of administration via any route of peptides to a subject. Pharmacokinetics can be described using methods and models, for example, compartmental models or noncompartmental methods.

Compartmental models can include but are not limited to monocompartmental model, the two compartmental model, the multicompartmental model, or the like. Models can be divided into different compartments and described by the corresponding scheme. For example, one scheme can be the absorption, distribution, metabolism and excretion (ADME) scheme. For another example, another scheme can be the liberation, absorption, distribution, metabolism and excretion (LADME) scheme. In some aspects, metabolism and excretion can be grouped into one compartment referred to as the elimination compartment. For example, liberation can include liberation of the active portion of the composition from the delivery system, absorption can include absorption of the active portion of the composition by the subject, distribution can include distribution of the composition through the blood plasma and to different tissues, metabolism, which can include metabolism or inactivation of the composition and finally excretion, which can include excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition can be biphasic, including, for example an alpha phase and a beta phase, or a gamma, delta or other phase can be observed.

Pharmacokinetics can include determining at least one parameter associated with administration of peptides to a subject. In some aspects, parameters can include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

### Use of Peptides as Imaging Agents

The present disclosure relates to peptides that that home, migrate to, distribute to, accumulate in, are directed to, and/or bind cancerous or diseased cells. These abilities make them useful for a variety of applications. For example, the peptides can have applications in site-specific modulation of biomolecules to which the peptides are directed. End uses of such peptides include, for example, imaging, research, therapeutics, pharmaceuticals, chemotherapy, chelation therapy, targeted drug delivery, and radiotherapy. Some uses can include targeted drug delivery and imaging.

In some embodiments, a peptide of the disclosure delivers a metal, a radioisotope, a dye, fluorophore, or another suitable material that can be used in imaging. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, ZW800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAN/IRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177 or another suitable material that can be used in imaging.

The present invention provides methods for intraoperative imaging and resection of a cancer, cancerous tissue, tumor tissue, or diseased or inflamed tissue using a peptide of the present disclosure conjugated with a detectable agent. In some aspects, the cancer, cancerous tissue, tumor tissue, or diseased or inflamed tissue or cells of the foregoing is detectable by fluorescence imaging that allows for intraoperative visualization of the cancer, cancerous tissue, tumor tissue, or diseased or inflamed tissue using a peptide of the present disclosure. In some aspects, the peptide of the present disclosure is conjugated to one or more detectable agents. For example, 1, 2, 3, 4, 5, 6, 7, 8, 9, or 10 detectable agents can be conjugated to a peptide of this disclosure. In a further embodiment, the detectable agent comprises a fluorescent moiety coupled to the peptide. In another embodiment, the detectable agent comprises a radionuclide. In some aspects, imaging is pre-operative imaging. In other aspects, imaging is achieved during open surgery. In further aspects, imaging is accomplished while using endoscopy or other non-invasive surgical techniques. In yet further aspects, imaging is performed after surgical removal of the cancer, cancerous tissue, tumor tissue, or diseased tissue or cells of the foregoing.

In some aspects, the present disclosure provides a method for detecting a cancer, cancerous tissue, tumor tissue or diseased tissue or cells of the foregoing, the method comprising the steps of contacting a tissue of interest with a peptide of the present disclosure, wherein the peptide is conjugated to a detectable agent and measuring the level of binding of the peptide, wherein an elevated level of binding is indicated by an increased detection of the detectable agent relative to normal tissue, which is indicative that the tissue is a cancer, cancerous tissue, tumor tissue or diseased tissue or cells of the foregoing. In some embodiments, the disclosure provides a method of imaging an organ or body region or region, tissue, or structure of a subject, the method comprising administering to the subject the peptide or pharmaceutical composition disclosed herein and imaging the subject. In some embodiments, such as those associated with cancers, the imaging can be associated with surgical removal of the diseased region, tissue, structure, or cell of the subject.

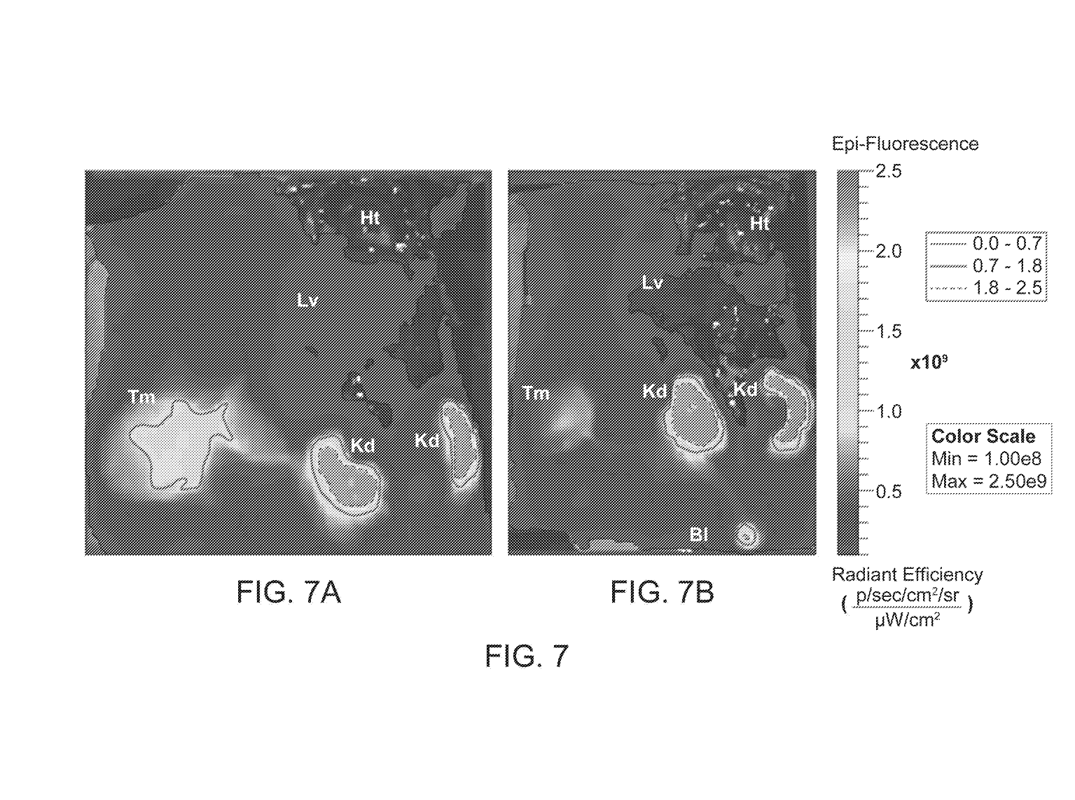

In certain embodiments, a peptide of SEQ ID NO: 1-SEQ ID NO: 35 or SEQ ID NO: 37-SEQ ID NO: 71 is conjugated to a detectable agent. In certain embodiments, a peptide of SEQ ID NO: 1-SEQ ID NO: 35 or SEQ ID NO: 37-SEQ ID NO: 71 is conjugated to a detectable agent and is administered in a subject in order to image the cancerous tissues and cells thereof. For example, a peptide of SEQ ID NO: 15 was conjugated to AlexaFluor647, and the peptide conjugate of SEQ ID NO: 15-A was administered to mice bearing A673 sarcoma flank tumors. After administration of the peptide conjugate, as shown in FIG. 7AB, whole body fluorescence imaging revealed signal corresponding to the peptide conjugate of SEQ ID NO: 15-A in tumor tissues.

### Use of Peptides in Treatment of Cancer

In one embodiment, the method includes administering an effective amount of a peptide as described herein to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide of the disclosure. The disease may be a cancer or tumor. In treating the disease, the peptide may contact the tumor or cancerous cells. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment can be provided to the subject for more than 1 day, 1 week, 1 month, 6 months, 12 months, 2 years or more after clinical onset of disease. Treatment can be provided to the subject for less than 1 day, 1 week, 1 month, 6 months, 12 months, or 2 years or more after clinical onset of the disease. Treatment can be administered daily, weekly, monthly, or yearly. Treatment can also include treating a human in a clinical trial. A treatment can comprise administering to a subject a pharmaceutical composition, such as one or more of the pharmaceutical compositions described throughout the disclosure. A treatment can comprise delivering a peptide of the disclosure to a subject, either intravenously, subcutaneously, intramuscularly, by inhalation, dermally, topically, orally, sublingually, intrathecally, transdermally, intranasally, via a peritoneal route, or directly into the brain, e.g., via and intracerebral ventrical route. A treatment can comprise administering a peptide-active agent complex to a subject, either intravenously, subcutaneously, intramuscularly, by inhalation, dermally, topically, orally, intrathecally, transdermally, intransally, parenterally, orally, via a peritoneal route, nasally, sublingually, or directly onto the cancerous tissues.

In some embodiments, the present disclosure provides a method for treating a cancer or tumor, the method comprising administering to a subject in need thereof an effective amount of a peptide as described herein. One example of cancers or conditions that can be treated with a peptide as described herein is solid tumors. Another example of cancers or conditions that can be treated with a peptide of the disclosure is sarcomas or Ewing sarcoma family of tumors. Further examples of cancers or conditions that can be treated with a peptide of the disclosure includes triple negative breast cancer, colon cancer, colon cancer metastases, acute lymphoblastic leukemia, acute myeloid leukemia, adrenocortical carcinoma, AIDS-related cancers such as Kaposi sarcoma, AIDS-related lymphoma, primary CNS lymphoma, anal cancer, appendix cancer, childhood astrocytomas, astrocytomas, childhood atypical teratoid/rhabdiod tumor, CNS atypical teratoid/rhabdiod tumor, atypical teratoid/rhabdiod tumor, basal cell carcinoma, skin cancer, bile duct cancer, bladder cancer, bone cancer, osteosarcoma, malignant fibrous histiocytoma, childhood brain stem glioma, brain stem glioma, brain tumor, brain and spinal cord tumors, central nervous system embryonal tumors, childhood central nervous system embryonal tumors, central nervous system germ cell tumors, childhood central nervous system germ cell tumors, craniopharyngioma, childhood craniopharyngioma, ependymoma, childhood ependymoma, breast cancer, bronchial tumors, childhood bronchial tumors, burkitt lymphoma, carcinoid tumor, gastrointestinal cancer, carcinoma of unknown primary, cardiac tumors, childhood cardiac tumors, primary lymphoma, cervical cancer, cholangiocarcinoma, chordoma, childhood chordoma, chronic lymphocytic leukemia, chronic myelogenous leukemia, chronic myeloproliferative neoplasms, colon cancer, colorectal cancer, cutaneous T cell lymphoma, ductal carcinoma in situ, endometrial cancer, esophageal cancer, esthesioneuroblastoma, childhood esthesioneuroblastoma, ewing sarcoma, extracranial germ cell tumor, childhood extracranial germ cell tumor, extragonadal germ cell tumor, eye cancer, intraocular melanoma, retinoblastoma, fallopian tube cancer, fibrous histiocytoma of bone, gallbladder cancer, gastric cancer, gastrointestinal carcinoid tumor, gastrointestinal stromal tumors, ovarian cancer, testicular cancer, gestational trophoblastic disease, glioma, hairy cell leukemia, head and neck cancer, hepatocellular cancer, histiocytosis, Langerhans cell histiocytosis, hodgkin lymphoma, hypopharyngeal cancer, intraocular melanoma, melanoma, islet cell tumors, pancreatic neuroendocrine tumors, kidney cancer, renal cell tumors, Wilms tumor, childhood kidney tumors, lip and oral cavity cancer, liver cancer, lung cancer, nonhodgkin lymphoma, macroglodulinemia, Waldenstrom macroglodulinemia, male breast cancer, merkel cell carcinoma, metastatic squamous neck cancer with occult primary, midline tract carcinoma involving NUT gene, mouth cancer, multiple endocrine neoplasia syndromes, childhood multiple endocrine neoplasia syndromes, multiple myeloma/plasma cell neoplasm, mycosis fungoides, myelodysplastic syndromes, myelodysplastic/myeloproliferative neoplasms, multiple myeloma, myloproliferative neoplasms, chronic myeloproliferative neoplasms, nasal cavity and paranasal sinus cancer, nasopharyngeal cancer, neuorblastoma, non-small cell lung cancer, oropharyngeal cancer, low malignant potential tumor, pancreatic cancer, pancreatic neuroendocrine tumors, papillomatosis, childhood papillomatosis, paraganglioma, paranasal sinus and nasal cavity cancer, parathyroid cancer, penile cancer, pheochromocytoma, pharyngeal cancer, pituitary tumor, pleuropulmonary blastoma, childhood pleuropulmonary blastoma, primary peritoneal cancer, prostate cancer, rectal cancer, pregnancy-related cancer, rhabdomyosarcoma, childhood rhabdomyosarcoma, salivary gland cancer, Sezary syndrome, small cell lung cancer, small intestine caner, soft tissue sarcoma, squamous cell carcinoma, testicular cancer, throat cancer, thymoma, thymic carcinoma, thyroid cancer, transitional cell cancer of the renal, pelvis, and ureter, uterine cancer, urethral cancer, endometrial cancer, uterine sarcoma, vaginal cancer, vascular tumors, and vulvar cancers.

In some embodiments, a peptide of the present disclosure exhibit protease inhibitor activity. For example, a peptide of the present disclosure can be a serine protease inhibitor. In certain embodiments, peptides are used to inhibit proteases of interest, such as coagulation-associated proteases (e.g., thrombin, factor 10a), metabolism-associated proteases (e.g., DPP-IV), cancer-associated proteases (e.g., matrix metalloproteinases, cathepsins), viral infection-associated proteases (e.g., HIV protease), and inflammation-associated proteases (e.g., tryptase, kallikrein).

In some aspects, the peptides of the present disclosure are conjugated to one or more therapeutic agents. In certain aspects, the therapeutic agent is a chemotherapeutic, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. In certain embodiments, a peptide of the present disclosure is conjugated to monomethyl auristatine E (MMAE), MMAF, auristatin, dolostatin, auristatin F, monomethylauristatin D, maytansinoid (e.g., DM-1, DM4, maytansine), pyrrolobenzodiazapine dimer, calicheamicin, N-acetyl-γ-calicheamicin, duocarmycin, anthracycline, a microtubule inhibitor, or a DNA damaging agent. For example, in certain embodiments, a peptide of the present disclosure is conjugated to MMAE, a non-specific cytotoxic drug, to direct drugs to cancerous cells.

Optionally, certain embodiments of the present disclosure provide peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cancer cells using both a therapeutic agent (e.g., drug) and electromagnetic energy (e.g., radiation or light) concurrently.

In certain embodiments, the peptide of the disclosure is mutated to home, distribute to, target, migrate to, accumulate in, or is directed to certain tissues but not to others, to change the strength or specificity of its function, or to gain or lose function, such as inhibiting a protease.

The present disclosure also encompasses the use of “tandem” peptides in which two or more peptides are conjugated or fused together. In certain embodiments, a tandem peptide comprises two or more knotted peptides conjugated or fused together, where at least one knotted peptide is capable of targeting to a specific region, while at least one other knotted peptide provides a specific therapeutic activity as discussed above and herein.

In some embodiments, the present disclosure provides a method for treating a cancer, the method comprising administering to a subject in need thereof an effective amount of a peptide of the present disclosure.

In some embodiments, the present disclosure provides a method for treating a cancer, the method comprising administering to a patient in need thereof an effective amount of a pharmaceutical composition comprising a peptide of the present disclosure and a pharmaceutically acceptable carrier.

In some embodiments, the present disclosure provides a method for inhibiting invasive activity of cells, the method comprising administering an effective amount of a peptide of the present disclosure to a subject.

A peptide comprising the sequence of any of SEQ ID NO: 1-SEQ ID NO: 35, SEQ ID NO: 37-SEQ ID NO: 71, or any peptide derivative or peptide-active agent as described herein, can be used to target sarcomas (e.g., Ewing's sarcoma). A peptide comprising the sequence of any of SEQ ID NO: 1-SEQ ID NO: 35, SEQ ID NO: 37-SEQ ID NO: 71, or any peptide derivative or peptide-active agent as described herein, can be used to target a sarcoma, cervical cancer, B cell lymphoma, breast cancer, brain cancer, Ewing sarcoma, Burkitt's lymphoma, medulloblastoma, rhabdomyosarcoma, or colorectal cancer. A peptide comprising the sequence of any of SEQ ID NO: 1-SEQ ID NO: 35, SEQ ID NO: 37-SEQ ID NO: 71, or any peptide derivative or peptide-active agent as described herein, can be used to target upper GI disease and cancers (e.g., throat, oral, esophageal cancer, salivary glands, tonsils, pharynx, adenosarcomas, oral malignant melanoma head and neck cancer). A peptide comprising the sequence of any of SEQ ID NO: 1-SEQ ID NO: 35, SEQ ID NO: 37-SEQ ID NO: 71, or any peptide derivative or peptide-active agent as described herein, can be used to additionally target gall bladder disease and cancers.

CTI and CTI peptide variants, conjugates, and pharmaceutical compositions described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the judgment of the treating physician.

In some embodiments, the present disclosure provides a method of treating a tumor or cancerous cells of a subject, the method comprising administering to the subject a peptide comprising the sequence of any of SEQ ID NO: 1-SEQ ID NO: 35 or SEQ ID NO: 37-SEQ ID NO: 71, or a functional fragment thereof, conjugated to an active agent. In some embodiments, the present disclosure provides a peptide conjugate comprising a peptide comprising the sequence of any of SEQ ID NO: 1-SEQ ID NO: 35 or SEQ ID NO: 37-SEQ ID NO: 71, or a functional fragment thereof, conjugated to an active agent.

Multiple peptides conjugated to active agents described herein can be administered in any order or simultaneously. In some cases, multiple functional fragments of peptides derived from CTI can be administered in any order or simultaneously. If simultaneously, the multiple peptides described herein can be provided in a single, unified form, such as an intravenous injection, or in multiple forms, such as subsequent intravenous dosages.

Peptide Kit

In one aspect, peptides described herein can be provided as a kit. In another embodiment, peptide conjugates described herein can be provided as a kit. In another embodiment, a kit comprises amino acids encoding a peptide described herein, a vector, a host organism, and an instruction manual. In some embodiments, a kit includes written instructions on the use or administration of the peptides.

### EXAMPLES

The following examples are included to further describe some aspects of the present disclosure, and should not be used to limit the scope of the invention.

### Example 1

### Manufacture of Peptides

This example describes the manufacture of the peptides described herein. Peptides derived from CTI knottin proteins were generated in mammalian cell culture using a published methodology (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques. (M. R. Green, Joseph Sambrook. Molecular Cloning. 2012 Cold Spring Harbor Press.). The resulting construct was packaged into a lentivirus, transfected into HEK293 cells, expanded, isolated by immobilized metal affinity chromatography (IMAC), cleaved with tobacco etch virus protease, and purified to homogeneity by reverse-phase chromatography. Following purification, each peptide was lyophilized and stored frozen.

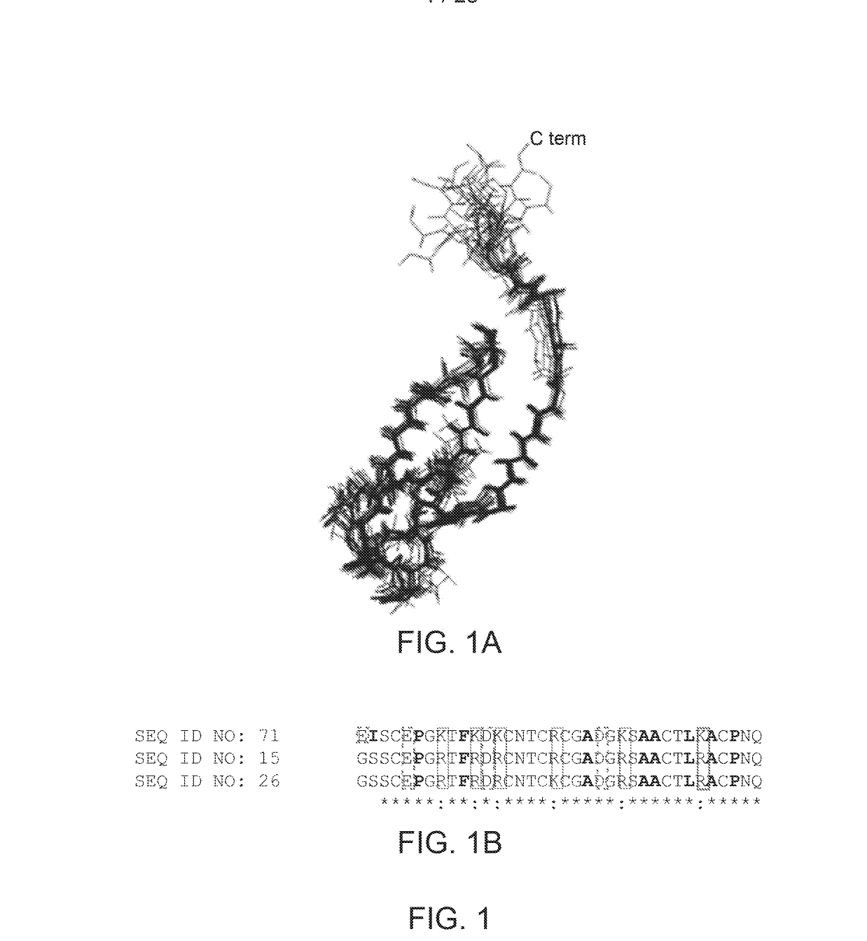

FIG. 1A shows a representation of chymotrypsin inhibitor II (CTI) and FIG. 1B shows the sequence of the CTI peptide of SEQ ID NO: 71 (EISCEPGKTFKDKCNTCRCGADGKSAACTLKACPNQ). FIG. 1B also shows the sequences of two example peptides of the disclosure including a peptide of SEQ ID NO: 15 and a peptide of SEQ ID NO: 26, which are variants of CTI.

### Example 2

### Peptide Expression Using a Mammalian Expression System

This example describes expression of the peptides using a mammalian expression system. Peptides were expressed according to the methods described in in Bandaranayake et al., Nucleic Acids Res. 2011 November; 39 (21): e143. Peptides were cleaved from siderocalin using tobacco etch virus protease and purified by FPLC on a hydrophobic columns using a gradient of acetonitrile and 0.1% TFA. Peptides were then lyophilized and stored frozen.

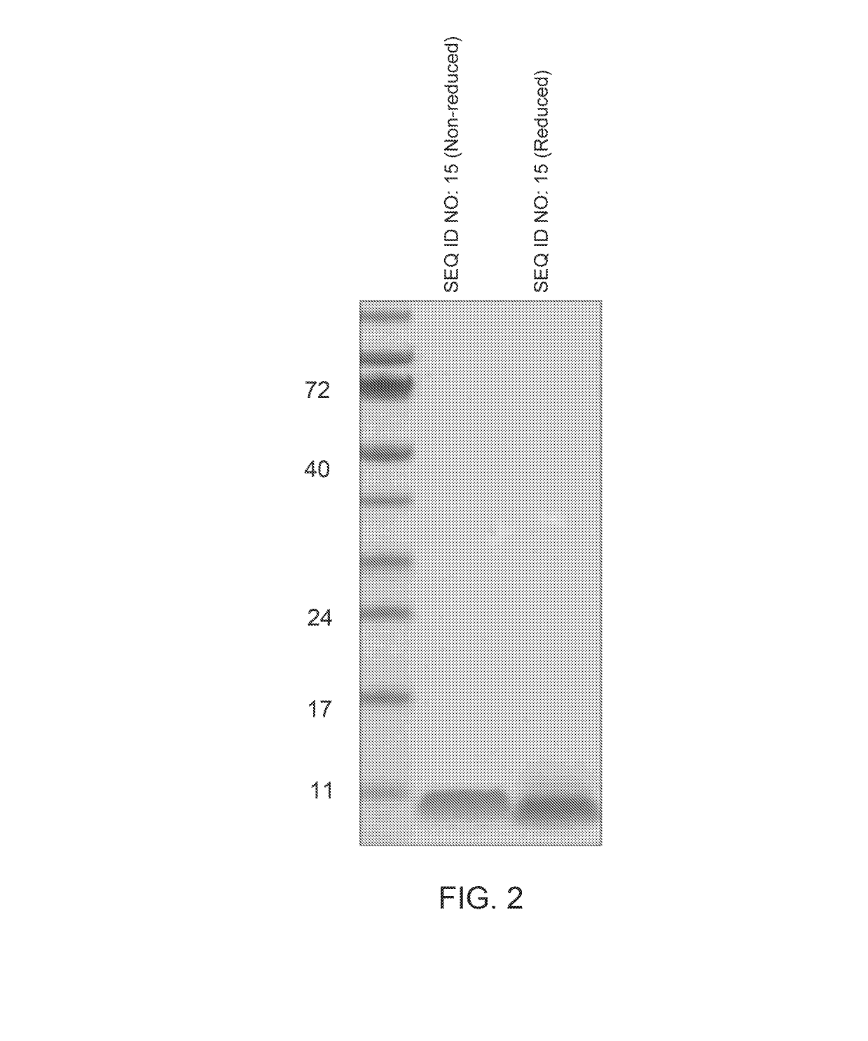

FIG. 2 nonreduced and reduced bands of a SEQ ID NO: 15 peptide on SDS-PAGE gels.

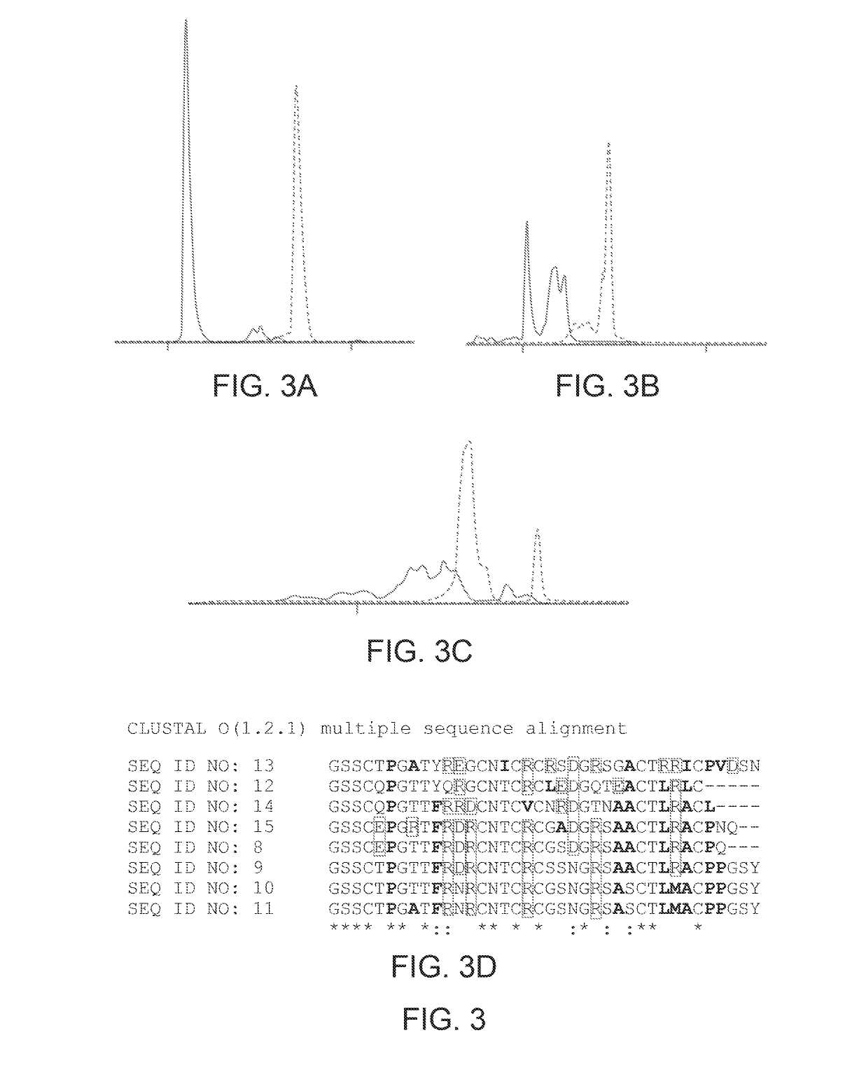

FIGS. 3A, 3B, and 3C show quality control data from small scale (30 mL) mammalian expression studies of the peptides of SEQ ID NO: 9, SEQ ID NO: 21, SEQ ID NO: 28. The graphs illustrate HPLC chromatograms on a hydrophobic column using a gradient of acetonitrile and 0.1% TFA. FIG. 3D illustrates a sequence comparison of SEQ ID NO: 13, SEQ ID NO: 12, SEQ ID NO: 14, SEQ ID NO: 15, SEQ ID NO: 8, SEQ ID NO: 9, SEQ ID NO: 10, and SEQ ID NO: 11. Solid traces show non-reduced proteins and dashed traces show reduced proteins.

### Example 3

### Peptide Dye Labeling

This example describes the dye labeling of peptides. A peptide of SEQ ID NO: 15 was expressed recombinantly and then the N-terminus of the peptide was conjugated to AlexaFluor647 (AF647) using an NHS ester of AF647 to produce a SEQ ID NO: 15-A peptide conjugate (SEQ ID NO: 15-A).

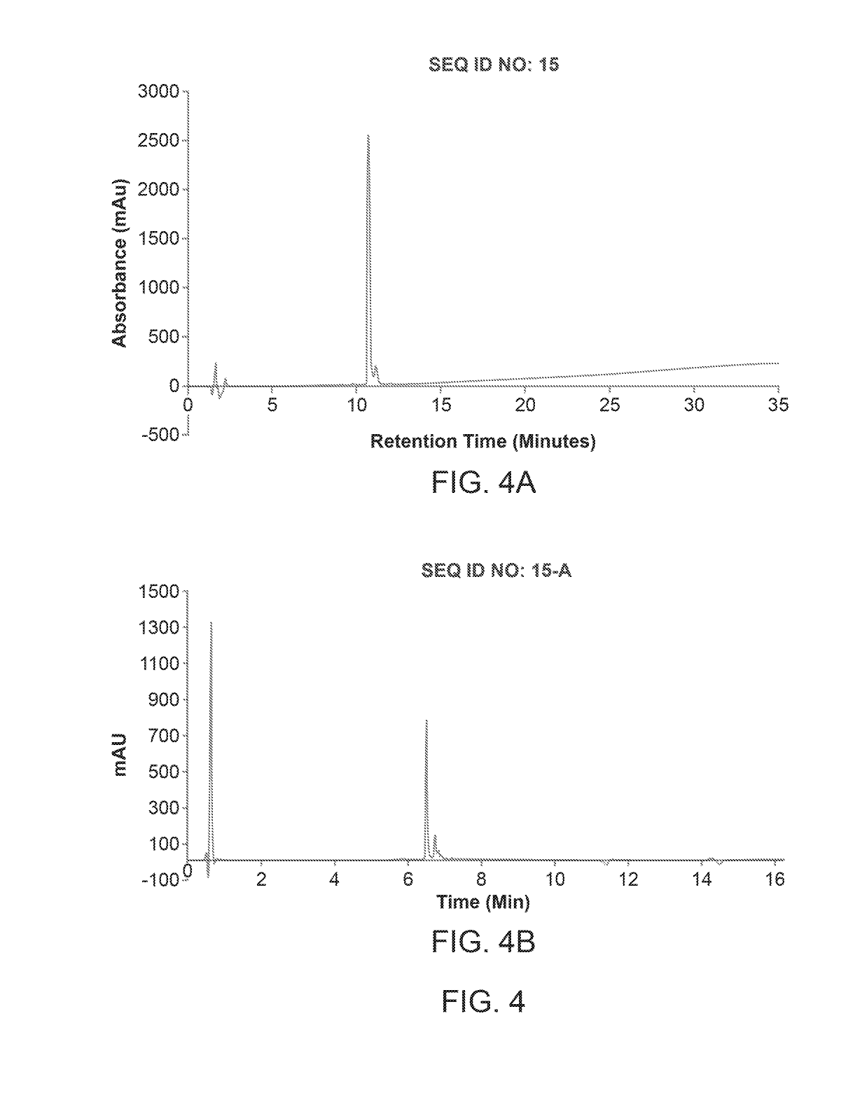

FIG. 4A shows HPLC chromatograms of a peptide of SEQ ID NO: 15 showing a single predominant peak. FIG. 4B shows HPLC chromatograms of SEQ ID NO: 15-A following dye conjugation showing a single predominant peak.

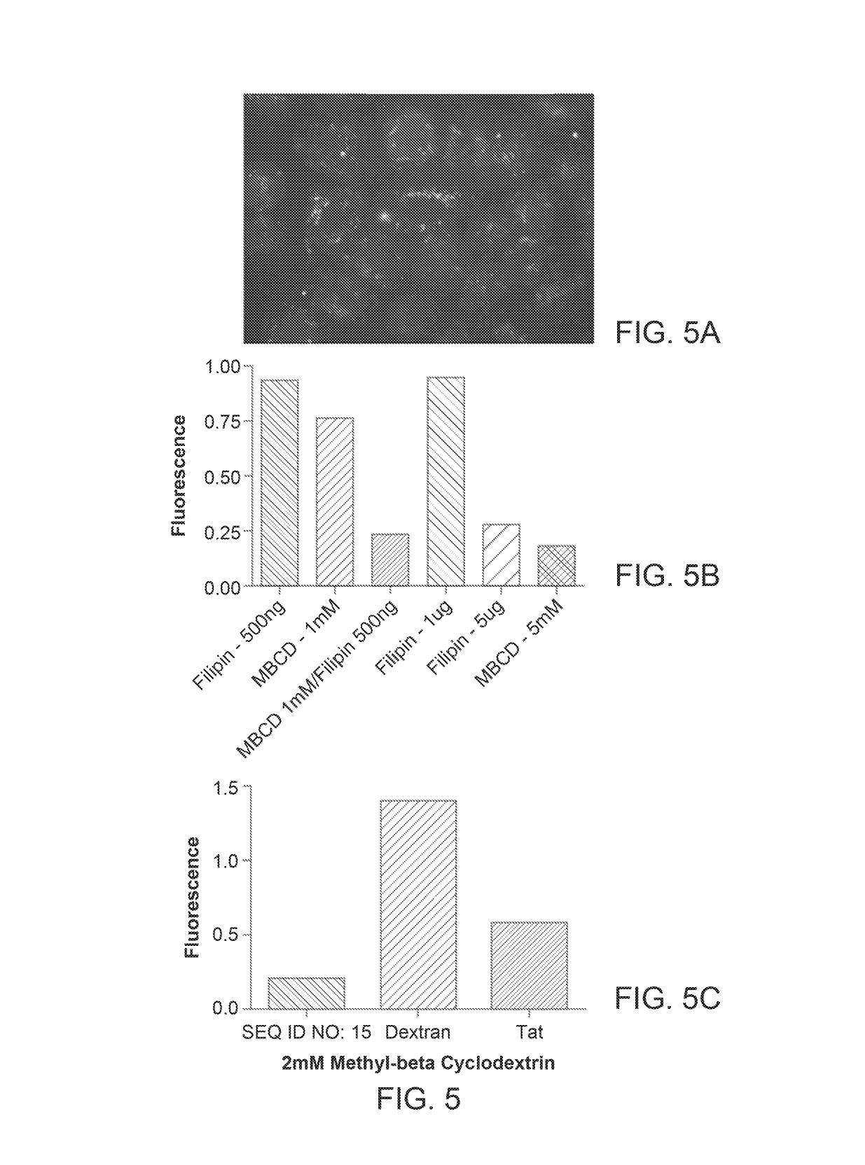

FIG. 5A shows a fluorescent image of SEQ ID NO: 15-A internalized by HeLa cells in different vesicles than the vesicles that internalized HIV-Tat-FITC. SEQ ID NO: 15-A and HIV-Tat-FITC, an HIV-Tat protein conjugated to a FITC fluorophore, were each dosed in HeLa cells at a concentration of 20 μM. The fluorescence image was taken on a Nikon Live microscope at 40× magnification, four hours after cells were dosed with SEQ ID NO: 15-A and HIV-Tat-FITC. The green fluorescence (FITC) and the red fluorescence (AF647) were located in different punctate regions, showing that the two molecules are localized in different vesicles.

FIG. 5B shows that endocytic inhibitors such as filipin and methyl-beta-cyclodextrin (MBCD) inhibit the uptake of SEQ ID NO: 15-A. SEQ ID NO: 15-A was dosed at concentration of 4 μM. Fluorescence was measured via flow cytometry and quantified to determine uptake of the peptide conjugate in cells.

FIG. 5C shows that 2 mM of MBCD inhibits uptake of SEQ ID NO: 15-A to a greater extent than dextran, a fluid-phase marker, or HIV-Tat. SEQ ID NO: 15-A was dosed at a concentration of 533 HIV-Tat-FITC was dosed at a concentration of 1 mM, and Dextran-Texas Red was dosed at a concentration of 25 mg/ml.

### Example 4

### Peptide Radiolabeling

This example describes the radiolabeling of peptides. Several knottins were radiolabeled by reductive methylation with ¹⁴C formaldehyde and sodium cyanoborohydride with standard techniques. The sequences were engineered to have the amino acids, “G”, “S” and “S” at the N terminus. See Methods in Enzymology V91:1983 p.570 and JBC 254(11):1979 p. 4359. An excess of formaldehyde was used to ensure complete methylation (dimethylation of every free amine). The labeled peptides were isolated via solid-phase extraction on Strata-X columns (Phenomenex 8B-S100-AAK), rinsed with water with 5% methanol, and recovered in methanol with 2% formic acid. Solvent was subsequently removed in a blowdown evaporator with gentle heat and a stream of nitrogen gas.

### Example 5

### Peptide Dosing for Tumor Homing

This example illustrates the dosing of peptides for homing to and accumulation in tumors. Two doses, 10 nmol and 53 nmol, of a SEQ ID NO: 15 peptide was conjugated to AF647 (SEQ ID NO: 15-A) were administered in female Harlan athymic nude mice bearing Ramos lymphoma flank tumor. Four hours following intravenous administration, mice were euthanized and tumors were dissociated. Tumor tissues were imaged using an IVIS system and single cell suspensions derived from tumor tissues were analyzed for fluorescence content by flow cytometry. Sterile water was delivered as a negative control to account for tumor tissue autofluorescence

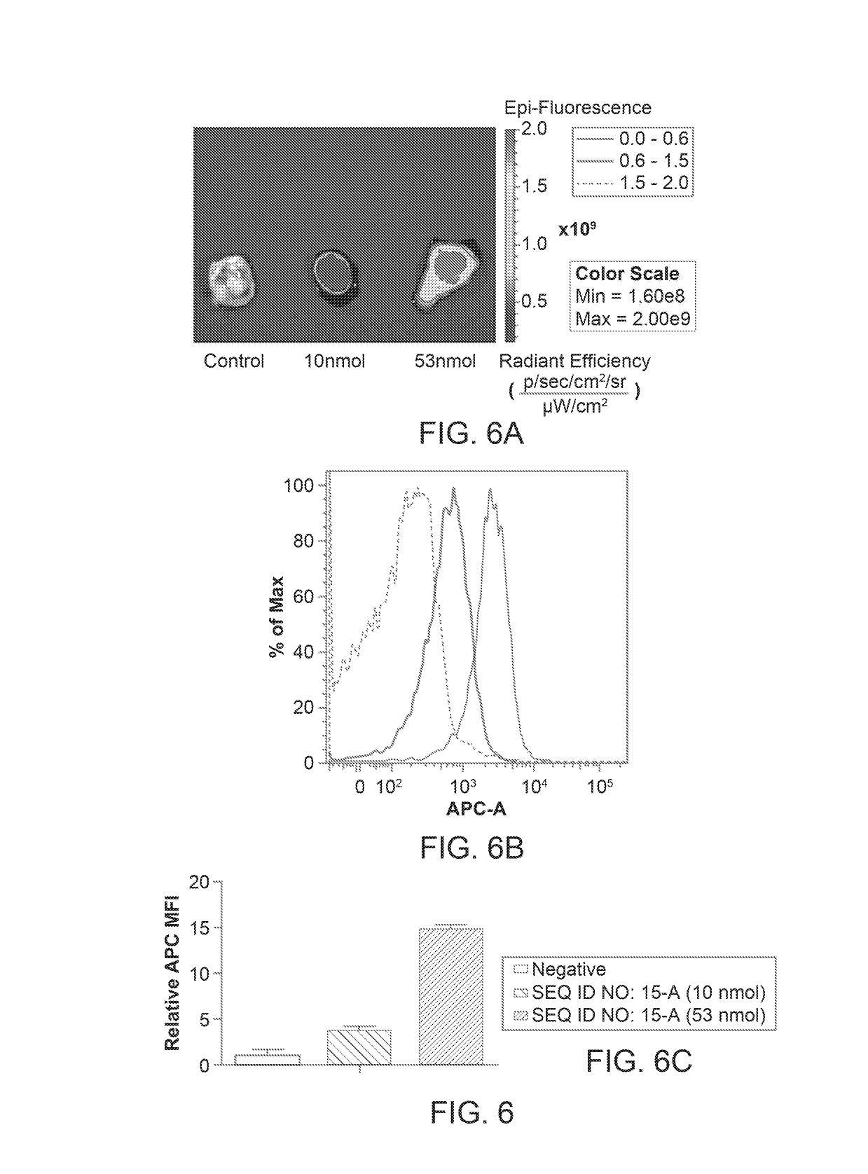

FIG. 6A shows distribution and accumulation of SEQ ID NO: 15-A in a dissociated tumor 4 hours following intravenous administration in a Ramos lymphoma tumor-bearing female Harlan athymic nude mouse. Control group (left) shows tumor tissue autofluorescence after administration of the negative control, and the SEQ ID NO: 15-A was administered at 10 nmol (middle) or 53 nmol doses (right). FIG. 6B shows flow cytometry of single cell suspensions derived from dissociated tumor tissues corresponding to FIG. 6A illustrating fluorescence in the negative control (left peak; dashed line), the 10 nmol of SEQ ID NO: 15-A (middle peak; dark line) dose, and the 53 nmol of SEQ ID NO: 15-A (right peak; light line) dose. FIG. 6C shows quantification of the relative mean fluorescence intensity (MFI) from flow cytometry data shown in FIG. 6B in the negative control, the 10 nmol SEQ ID NO: 15-A peptide conjugate dose, and the 53 nmol SEQ ID NO: 15-A dose.

### Example 6

### Peptide Homing to Tumors

This example describes peptide homing to and accumulation in tumors. A peptide of SEQ ID NO: 15 was expressed recombinantly and then the N-terminus of the peptide was conjugated to AlexaFluor647 (AF647) using an NHS ester AF647, producing SEQ ID NO: 15-A peptide conjugate (SEQ ID NO: 15-A).

A target dosage of 10 nmol SEQ ID NO: 15-A was administered to separate flank A673 Ewing's Sarcoma tumor bearing Female Harlan athymic nude mice while anesthetized. Each peptide-conjugate was allowed to freely circulate within the animal for 4 hours before the animals were euthanized. In vivo fluorescence images were captured using an IVIS Spectrum showing peptide distribution to various organs including the heart, liver, kidneys, bladder, and tumor. The tumors, brain, colon, skin tissue, liver, spleen, muscle tissue, and kidney were excised from each animal, and imaged using an IVIS Spectrum. Tumor fluorescence in dissociated tumors was quantified by defining a region of interest (ROI) and calculating the average radiant efficiency.

FIG. 7A shows a fluorescence image illustrating in vivo biodistribution of SEQ ID NO: 15-A in a female Harlan athymic mouse bearing A673 flank tumor xenografts 4 hours after administering 10 nmol of SEQ ID NO: 15-A peptide conjugate. Organs visualized in this image include liver (Lv), tumor (Tm), kidney (Kd), bladder (Bl), and heart (Ht). FIG. 7B shows a whole body fluorescence image illustrating in vivo biodistribution of SEQ ID NO: 15-A in a female Harlan athymic mouse different than the mouse shown in FIG. 7A, bearing A673 flank tumor xenografts 4 hours after administrating 10 nmol of SEQ ID NO: 15-A. Organs visualized in this image include liver (Lv), tumor (Tm), kidney (Kd), bladder (Bl), and heart (Ht).

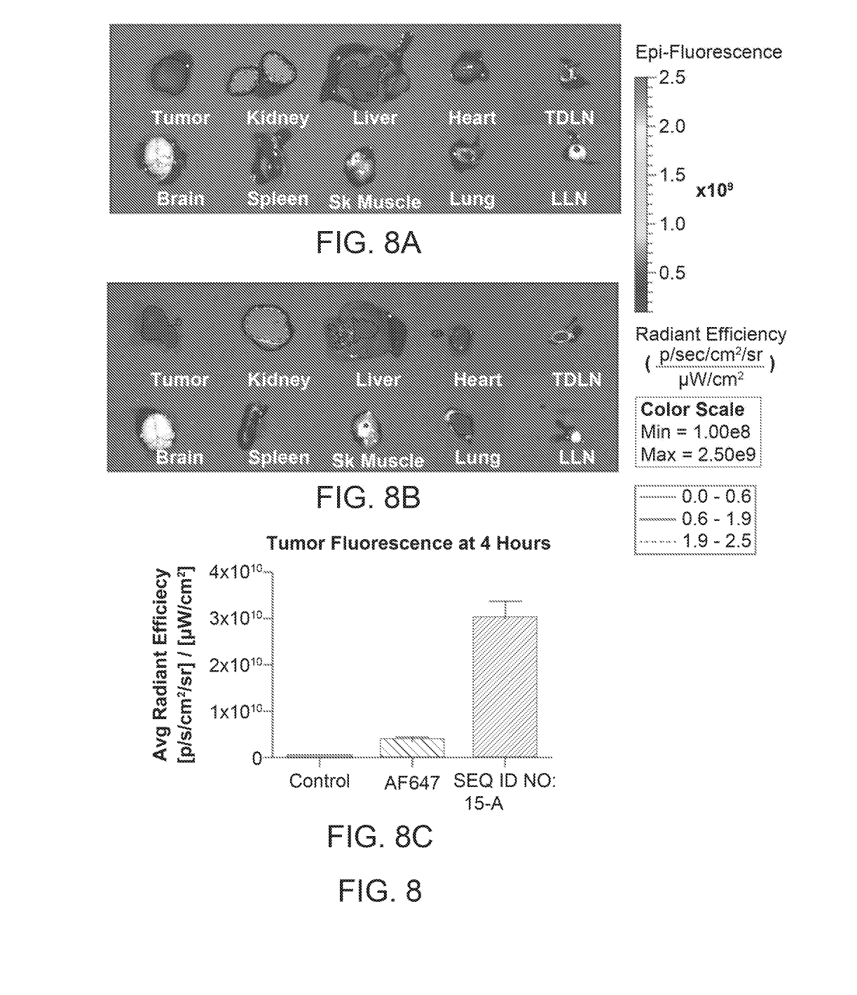

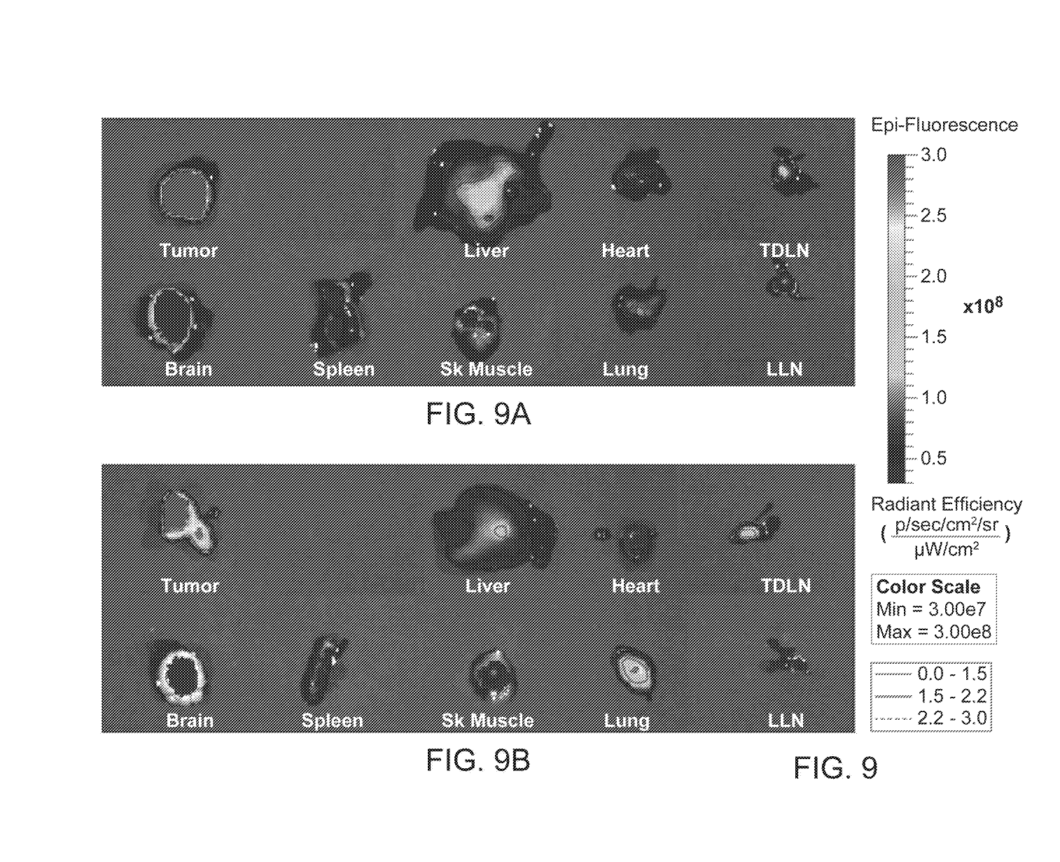

FIGS. 8A & 8B show ex vivo fluorescence images illustrating biodistribution and accumulation of SEQ ID NO: 15-A 4 hours after administration of 10 nmol SEQ ID NO: 15-A to different female Harlan athymic mice bearing an A673 Ewing's sarcoma flank tumor, in ten organs including tumor, kidney, liver, heart, tumor-draining lymph nodes (TDLN), brain, spleen, skeletal muscle, lungs, and lumbar lymph nodes (LLN). Fluorescence signal of SEQ ID NO: 15-A is high in the tumor and kidneys, compared to other organs. FIGS. 9A & 9B show the same tissues corresponding FIGS. 8A & 8B, except the kidneys were removed from the field before imaging.

FIG. 8C shows quantification of the average radiant efficiency in tumor tissues ex vivo after administration of 10 nmol AlexaFluor647 (AF647), and 10 nmol SEQ ID NO: 15-A 4 hours after administration to a female Harlan athymic mouse bearing an A673 Ewing's sarcoma flank tumors (n=3-5 per group). Negative controls show tissues from mice in which nothing was administered. Fluorescence signal of SEQ ID NO: 15-A was high in the tumor compared to negative controls.

### Example 7

### Whole-Body Autoradiography and Biodistribution after Peptide Administration

This example illustrates whole-body autoradiography showing peptide accumulation in tumors after administration of peptides. RH-28 tumor-bearing female Harlan athymic nude mice were given different dosages of the radiolabeled peptides or radiolabeled peptide conjugates. The kidneys of each mouse were either left intact or ligated to prevent renal filtration of the peptides. The peptides were radiolabeled by methylating lysines and the N-terminus so the actual binding agent may contain methyl or dimethyl lysine(s) and a methylated or dimethylated amino terminus. The radiolabeled peptides were radiolabeled SEQ ID NO: 26 peptide (SEQ ID NO: 26-r). The peptides were dye labeled by conjugating AlexaFluor647 (AF647) to a free amine on either the N-terminus or lysine with an NHS ester. The dye labeled peptides were radiolabeled SEQ ID NO: 26-A peptide conjugates (SEQ ID NO: 26-Ar).

A target dosage of 12 nmol SEQ ID NO: 26 peptide or 12 nmol SEQ ID NO: 26-Ar carrying 2 uCi of ¹⁴C was administered to Female Harlan athymic nude mice with intact kidneys and RH-28 tumor while anesthetized. Mice were euthanized 24 hours after administration of the peptide conjugates.

At the end of the dosing period, the mice were frozen in a hexane/dry ice bath and then frozen in a block of carboxymethylcellulose. Whole mouse sagittal slices were prepared that resulted in thin frozen sections being available for imaging. Thin, frozen sections of the mice were obtained with a microtome, allowed to desiccate in a freezer, and exposed to phosphoimager plates for about ten days.

These plates were developed, and the signal (densitometry) from each organ was normalized to the signal found in the heart blood of each mouse. A signal in tissue darker than the signal expected from blood in that tissue indicates peptide accumulation in a region, tissue, structure or cell.

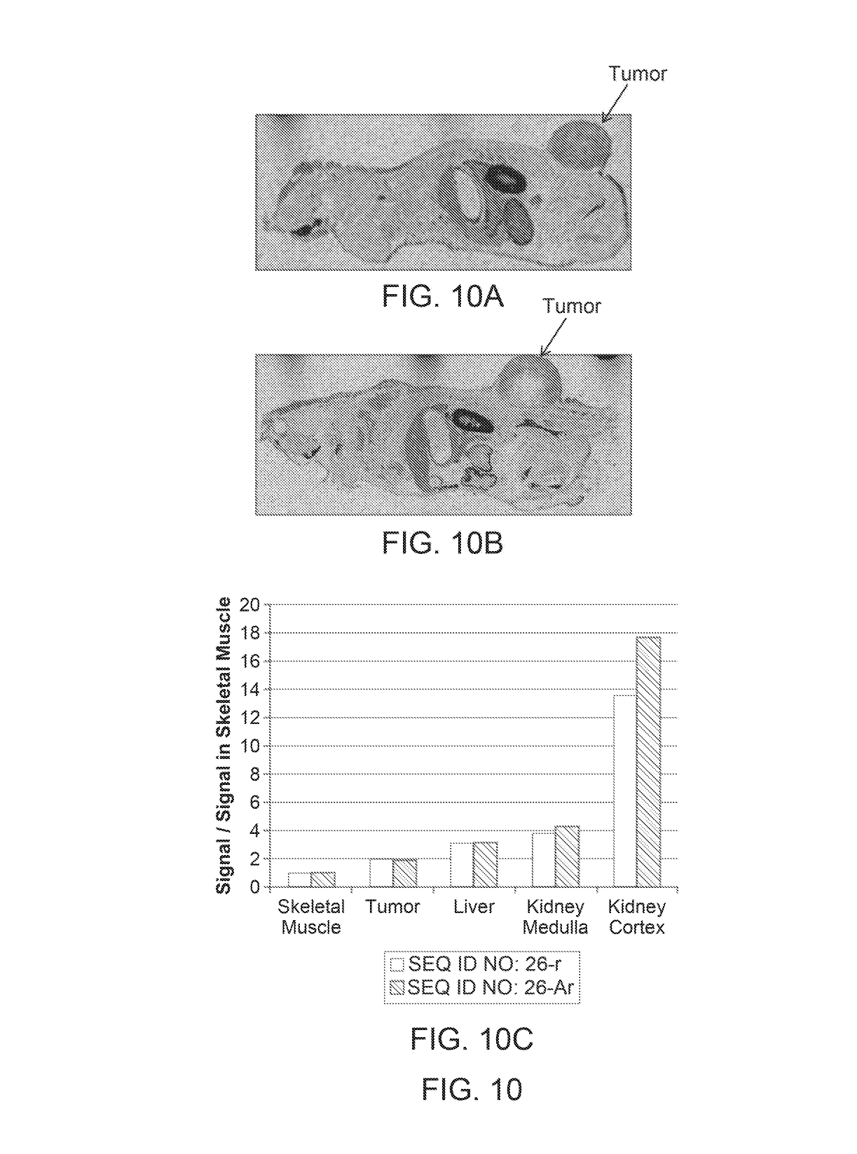

FIG. 10A shows an autoradiographic image in which the ¹⁴C signal identifies the peptide distribution in the tissues, including the RH-28 tumor of a mouse 24 hours after administration of 12 nmol of SEQ ID NO: 26-r. In these autoradiographic images, the ¹⁴C signal identifies the peptide distribution in tissues, including the RH-28 tumor of a mouse. FIG. 10B shows an autoradiographic image in which the ¹⁴C signal identifies the peptide distribution in the tissues, including the RH-28 tumor of a mouse 24 hours after administration 12 nmol of SEQ ID NO: 26-Ar.

FIG. 10C shows quantitation of ¹⁴C labeled peptides of SEQ ID NO: 26 (SEQ ID NO: 26-r) and SEQ ID NO: 26-Ar in various tissues including skeletal muscle, tumor, liver, kidney medulla, and kidney cortex, 24 hours after administration. Signal in each tissue was normalized to the signal in skeletal muscle.

These data indicate that a peptide or a peptide conjugated to an active agent or detectable agent accumulate in tumors.

### Example 8

### Whole-Body Autoradiography and Caspase-3 Staining after Peptide-MMAE Conjugate Administration

This example illustrates whole-body autoradiography and Caspase-3 staining after administration of peptide conjugated to MMAE. MMAE was conjugated to a peptide of SEQ ID NO: 26 with a Val-Cit-PABC linker to produce a SEQ ID NO: 26-B peptide conjugate (SEQ ID NO: 26-B). 7.5 nmol MMAE or 7.5 nmol SEQ ID NO: 26-B were administered to RH-28 tumor-bearing or Ramos lymphoma tumor-bearing female Harlan athymic mice and allowed to freely circulate for 24 or 48 hours.

At the end of the dosing period, mice were frozen in a hexane/dry ice bath and then frozen in a block of carboxymethylcellulose. Whole mouse sagittal slices were prepared that resulted in thin frozen sections being available for imaging. Thin, frozen sections of mice were obtained with a microtome, allowed to desiccate in a freezer, and exposed to phosphoimager plates for about ten days.

These plates were developed, and the signal (densitometry) from each organ was normalized to the signal found in the heart blood of each mouse. A signal in tissue darker than the signal expected from blood in that tissue indicates peptide accumulation in a region, tissue, structure or cell.

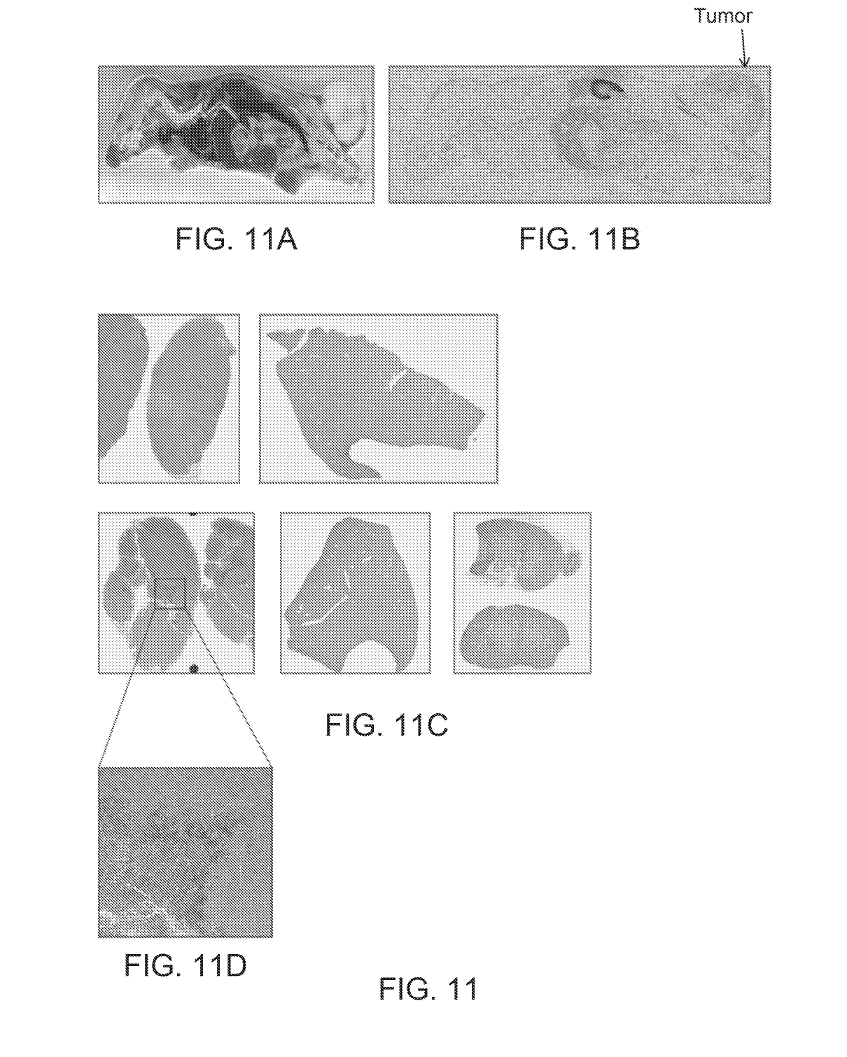

FIG. 11A shows a white light image of a frozen section of a mouse bearing an RH-28 tumor 24 hours after administration of 14 nmol of radiolabeled peptide of SEQ ID NO: 26 conjugated to monomethyl auristatin E (MMAE) (SEQ ID NO: 26-Br peptide conjugate). FIG. 11B shows an autoradiographic image corresponding to FIG. 11A in which the ¹⁴C signal identifies the peptide distribution in the tissues, including accumulation in the RH-28 tumor, of a mouse 24 hours after administration of 14 nmol SEQ ID NO: 26-Br. Separately, Ramos lymphoma tumor tissue from mice were stained for Caspase-3 to identify Caspase-3 activation in tissues. FIG. 11C shows Caspase-3 staining in tumor (left column), liver (middle column), and kidney (right column) of a mouse 48 hours after administration of 7.5 nmol MMAE (top row) or 7.5 nmol SEQ ID NO: 26-Br (bottom row). FIG. 11D shows a magnification of Caspase-3 staining in Ramos lymphoma tumor tissues corresponding to FIG. 11C (bottom left) after administration of 7.5 nmol SEQ ID NO: 26-Br.

The Caspase-3 staining, including the staining in the tumor, indicates induction of apoptosis by MMAE administration to tissues.

### Example 9

### Viability of Cell Lines after Administration of MMAE or MMAE-Peptide Conjugate

This example describes the viability different cell lines to MMAE or MMAE-peptide conjugates and toxicity induced by delivery of MMAE to cells by a peptide conjugate of the disclosure. A peptide of SEQ ID NO: 26 was expressed recombinantly or chemically synthesized and then conjugated to a therapeutic agent, MMAE, by a Val-Cit-PAB linker, producing SEQ ID NO: 26-B peptide conjugate (SEQ ID NO: 26-B). HeLa cells were treated in vitro with MMAE, SEQ ID NO: 26 peptide, or SEQ ID NO: 26-B, and viability was quantified. Ewing's Sarcoma cell lines including the RH28 cell line, A673 cell line, and A204 cell line, were treated in vitro with MMAE or SEQ ID NO: 26-B for 48-72 hours and viability of cells was quantified.

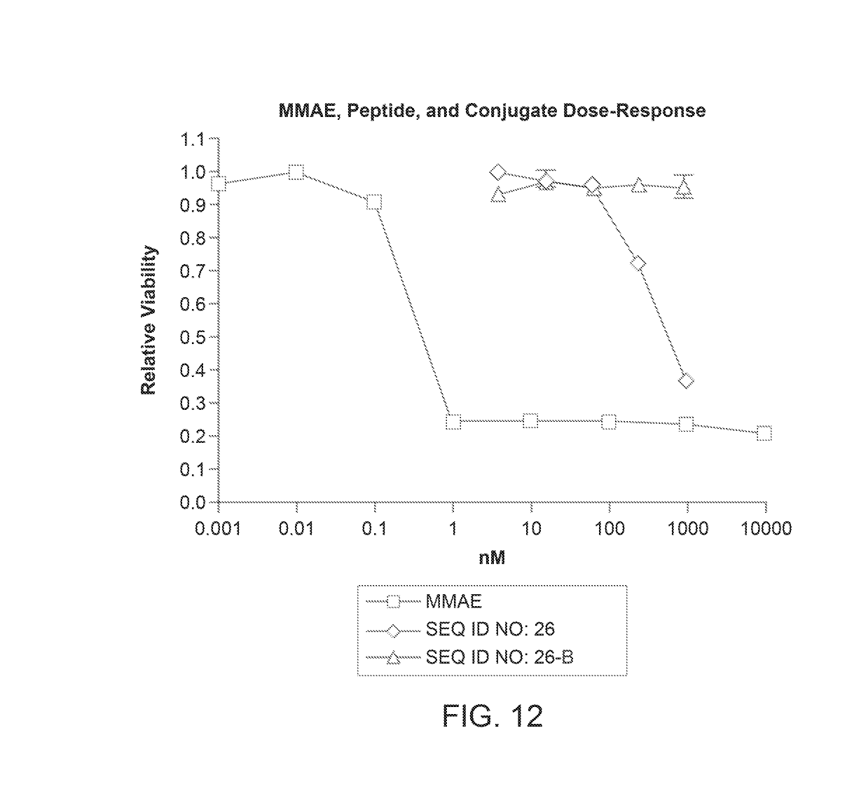

FIG. 12 illustrates relative viability of HeLa cells treated for 48 hours with various doses of MMAE, SEQ ID NO: 26 peptide, or SEQ ID NO: 26-B.

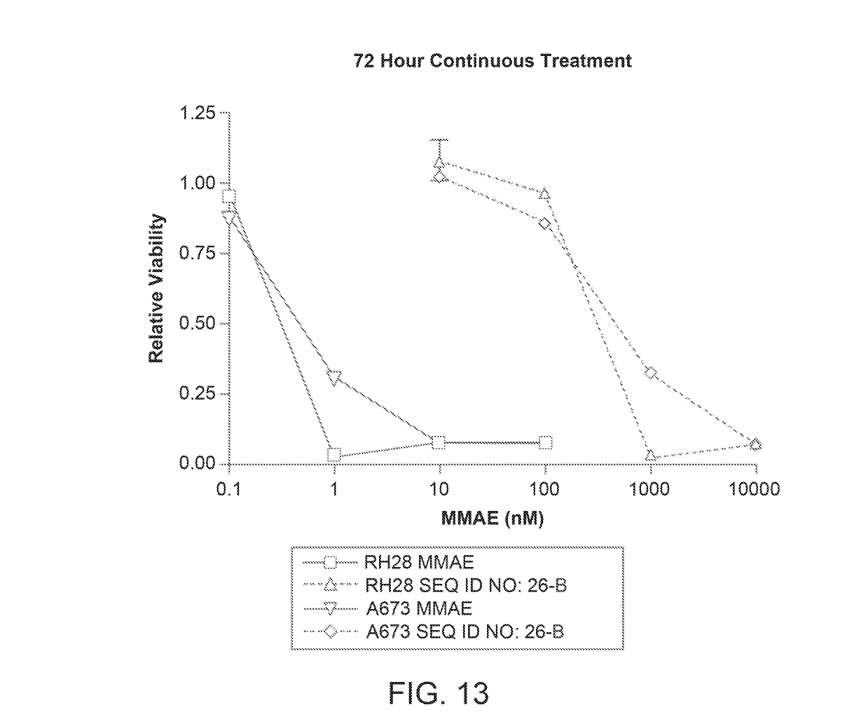

FIG. 13 illustrates relative viability of RH28 or A673 sarcoma cell lines after 72 hours of continuous treatment of various doses of MMAE or SEQ ID NO: 26-B. SEQ ID NO: 26-B inhibited growth of both cell lines.

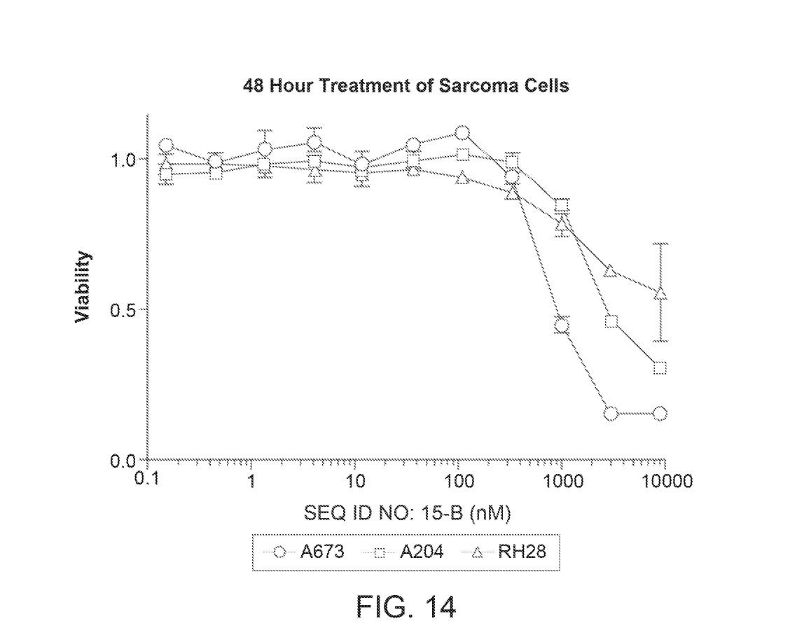

FIG. 14 illustrates relative viability of A673, A204, and RH28 sarcoma cell lines 48 hours after administration of various doses of SEQ ID NO: 15-B. SEQ ID NO: 15-B inhibited growth of all three cell lines.

### Example 10

### Modifying a Function of a Peptide of the Disclosure

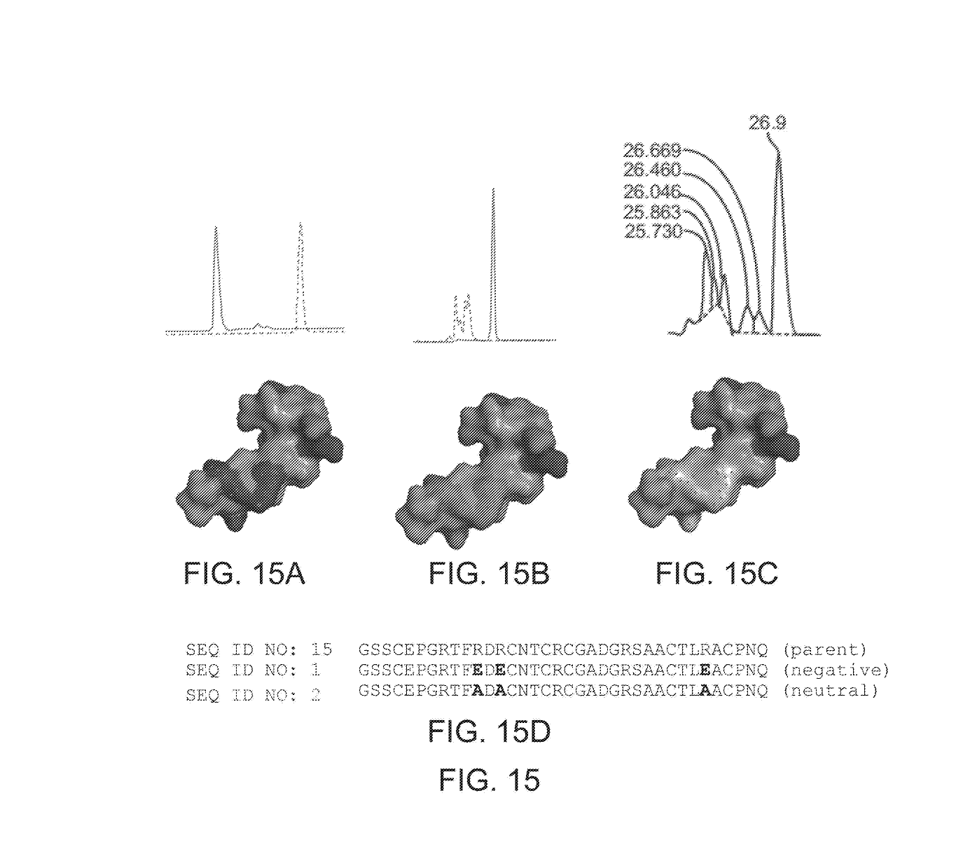

This example illustrates the modification of a function of a peptide. A peptide of SEQ ID NO: 15 was modified at positively charge patches to give a peptide of SEQ ID NO: 1 and a peptide of SEQ ID NO: 2. The modified peptides were expressed recombinantly. Mutations at positively charged patches of a peptide of SEQ ID NO: 15 were not well tolerated as indicated by the lack of folding purity indicated by the HPLC traces in FIG. 15. This indicated that the presence of positive residues in the mutated positions were important for function.

FIG. 15A shows HPLC traces of a peptide of SEQ ID NO: 15 where solid traces show protein reduced with dithiothretiol (DTT) and dashed traces show non-reduced proteins. Below each HPLC trace is the corresponding model of a peptide of SEQ ID NO: 15, where dark gray regions indicate regions of positive charge, medium-colored gray regions indicate regions of negative charge, and light gray regions indicate regions of neutral charge. FIG. 15B shows HPLC traces of a peptide of SEQ ID NO: 1 where solid traces show protein reduced with DTT and dashed traces show non-reduced proteins. Below each HPLC trace is the corresponding model of a peptide of SEQ ID NO: 1, where dark gray regions indicate regions of positive charge, medium-colored gray regions indicate regions of negative charge, and light gray regions indicate regions of neutral charge. FIG. 15C shows HPLC traces of a peptide of SEQ ID NO: 2 where solid traces show protein reduced with DTT and dashed traces show non-reduced proteins. Below each HPLC trace is the corresponding model of a peptide of SEQ ID NO: 2, where dark gray regions indicate regions of positive charge, medium-colored gray regions indicate regions of negative charge, and light gray regions indicate regions of neutral charge. FIG. 15D shows sequences of peptides of SEQ ID NO: 15, SEQ ID NO: 1, and SEQ ID NO: 2.

### Example 11

### Peptide Administration with Detectable Agents

This example describes peptide administration with detectable agents. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. The peptide is then conjugated to a detectable agent, such as a fluorophore, a near-infrared dye, a contrast agent, a nanoparticle, a metal-containing nanoparticle, a metal chelate, an X-ray contrast agent, a PET agent, a radioisotope, a radiosensitizer, or a radionuclide chelator, directly or via a cleavable or noncleavable linker.

One or more detectable agent-peptide conjugates are administered to a subject. The subject is a human or an animal.

### Example 12

### Treatment of a Cancer with a Peptide Conjugate of the Disclosure

This example describes the use of the peptides of the disclosure to treat a cancer. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, capecitabine, fluorouracil, irinotecan, oxaliplatin, a DNA damaging agent, or teniposide.

Peptide conjugation to a chemotherapeutic agent is direct or via a cleavable or noncleavable linker. Coupling of the chemotherapeutic agent to a peptide of the disclosure targets the drug to the cancer.

One or more chemotherapeutic agent-peptide conjugates are administered to a subject in need thereof. The subject is a human or an animal.

### Example 13

Treatment of Ewing's Sarcoma with a Peptide Conjugate of the Disclosure

This example describes the use of the peptides of the disclosure to treat Ewing's Sarcoma. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide

Peptide conjugation to a chemotherapeutic agent is direct or via a cleavable or noncleavable linker. Coupling of the chemotherapeutic agent to a peptide of the disclosure targets the drug to Ewing's Sarcoma.

One or more chemotherapeutic agent-peptide conjugates are administered to a subject in near thereof. The subject is a human or an animal.

### Example 14

Detection of Cancer with a Peptide Conjugate of the Disclosure

This example describes detection of cancer with a peptide conjugate of the disclosure. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some cancer detection methods, the peptide is fucosylated. In some cancer detection methods, the fucosylation is at Thr9. In some cancer detection methods, the fucosylation is at Thr7. In other cancer detection methods, the peptide is not fucosylated. The peptide is then conjugated to a detection agent, such as a fluorophore, a near-infrared dye, a contrast agent, a nanoparticle, a metal-containing nanoparticle, a metal chelate, an X-ray contrast agent, a PET agent, a radioisotope, a radiosensitizer, or a radionuclide chelator, directly or via a cleavable or noncleavable linker.

Coupling of the detection agent to a peptide of the disclosure targets the drug to the tumor.

One or more detectable agent-peptide conjugates are administered to a subject. The subject is a human or an animal. The cancer is detected by the detectable agent-peptide conjugate.

### Example 15

Treatment of Cervical Cancer with a Peptide Conjugate of the Disclosure

This example describes the use of the peptides of the disclosure to treat cervical cancer. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide

Peptide conjugation to a chemotherapeutic agent is direct or via a cleavable or noncleavable linker. Coupling of the chemotherapeutic agent to a peptide of the disclosure targets the drug to cervical cancer.

One or more chemotherapeutic agent-peptide conjugates are administered to a subject in near thereof. The subject is a human or an animal.

### Example 16

### Treatment of B Cell Lymphoma with a Peptide Conjugate of the Disclosure

This example describes the use of the peptides of the disclosure to treat B cell lymphoma. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide

Peptide conjugation to a chemotherapeutic agent is direct or via a cleavable or noncleavable linker. Coupling of the chemotherapeutic agent to a peptide of the disclosure targets the drug to cancerous B cells.

One or more chemotherapeutic agent-peptide conjugates are administered to a subject in near thereof. The subject is a human or an animal.

### Example 17

### Treatment of Medulloblastoma with a Peptide Conjugate of the Disclosure

This example describes the use of the peptides of the disclosure to treat medulloblastoma. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide

Peptide conjugation to a chemotherapeutic agent is direct or via a cleavable or noncleavable linker. Coupling of the chemotherapeutic agent to a peptide of the disclosure targets the drug to cancerous medulloblastoma cells.

One or more chemotherapeutic agent-peptide conjugates are administered to a subject in near thereof. The subject is a human or an animal.

### Example 18

### Treatment of Burkitt's Lymphoma with a Peptide Conjugate of the Disclosure

This example describes the use of the peptides of the disclosure to treat Burkitt's lymphoma. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide

Peptide conjugation to a chemotherapeutic agent is direct or via a cleavable or noncleavable linker. Coupling of the chemotherapeutic agent to a peptide of the disclosure targets the drug to cancerous Burkitt's lymphoma cells.

One or more chemotherapeutic agent-peptide conjugates are administered to a subject in near thereof. The subject is a human or an animal.

### Example 19

### Treatment of Rhabdomyosarcoma with a Peptide Conjugate of the Disclosure

This example describes the use of the peptides of the disclosure to treat rhabdomyosarcoma. A peptide of the disclosure (e.g., any of the peptides of SEQ ID NO: 1-SEQ ID NO: 35 or SEQ ID NO: 37-SEQ ID NO: 71) is expressed recombinantly or chemically synthesized. In some treatments, the peptide is fucosylated. In some treatments, the fucosylation is at Thr9. In some treatments, the fucosylation is at Thr7. In other treatments, the peptide is not fucosylated. The peptide is then conjugated to a chemotherapeutic agent, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide

Peptide conjugation to a chemotherapeutic agent is direct or via a cleavable or noncleavable linker. Coupling of the chemotherapeutic agent to a peptide of the disclosure targets the drug to cancerous rhabdomyosarcoma cells.

One or more chemotherapeutic agent-peptide conjugates are administered to a subject in near thereof. The subject is a human or an animal.

### Example 20

### Pharmacokinetics of a Peptide of the Disclosure

This example describes analysis of the pharmacokinetics of a peptide of the disclosure. A ¹⁴C radiolabeled peptide of SEQ ID NO: 15 (SEQ ID NO: 15-r) was administered to 6-10 week old female Harlan athymic mice at a dose of 2 μCi/33 nmol via intravenous administration, intraperitoneal administration, oral administration or subcutaneous administration. At each time point, urine was collected by massaging the abdomen and mice were subsequently euthanized by CO₂ asphyxiation. Blood was collected by cardiac exsanguination following cessation of respiratory movement. Plasma was separated by centrifugation and frozen until further analysis. Radioactivity in biological samples (plasma or urine) was detected by liquid scintillation counting on a Beckman Scintillation counter at a read time of 10 minutes. For HPLC evaluation, urine and plasma samples were diluted 20 μl in 80 μl and analyzed on an Agilent analytical HPLC equipped with an inline Raytest scintillation detector. Pharmacokinetics analysis was performed using the PKSolver 2 compartment IV or extravascular bolus.

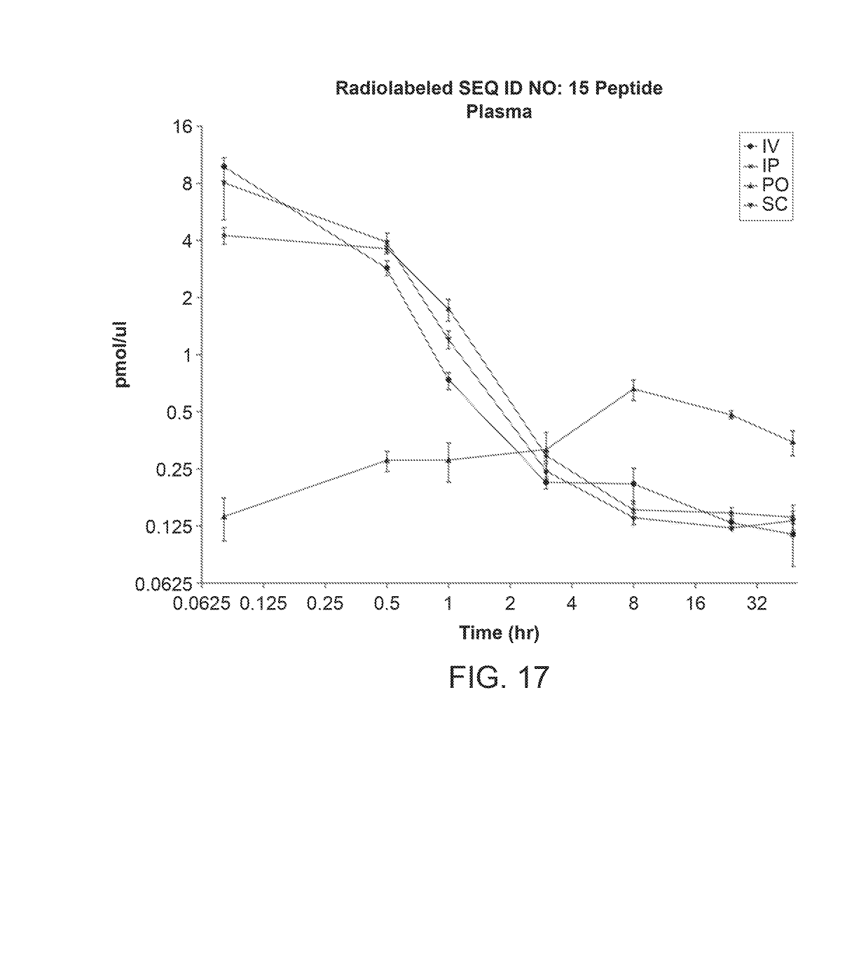

FIG. 17 shows liquid scintillation counting and quantification of the concentration of SEQ ID NO: 15-r recovered in plasma at several time points after administration of 2 μCi/33 nmol of SEQ ID NO: 15-r in female Harlan athymic nude mice via intravenous (IV) administration shown in circle data points, intraperitoneal (IP) administration shown in square data points, oral (PO) administration shown in triangle data points, and subcutaneous (SC) administration shown in inverted triangle data points. Each data point shows mean and error bars indicating standard deviation (n=3).

TABLE 2 shows a summary of pharmacokinetic parameters after administration of 2 μCi/33 nmol of SEQ ID NO: 15-r in female Harlan athymic nude mice via different routes, including T_{α}^{1/2} life (elimination half-life, min), Tₘₐₓ (time to reach Cₘₐₓ, min), Cₘₐₓ (peak drug concentration, pmol/μl), CL/F (drug clearance over time, nmol/(pmol/μl)/h), AUC_{0-inf} (area under the curve, pmol/μl*h²), and R².

TABLE 2
Pharmacokinetic Parameters of IV, IP,
SC, and Oral Routes of Administration

| | IV | IP | SC | Oral | |
|---|---|---|---|---|---|
| T_{α} ½ life (min) | 13.2 | 25.8 | 16.32 | 28 | hr |
| Tₘₐₓ (min) | 0 | 10.9 | 7.38 | 8.5 | hr |

| | | | | |
|---|---|---|---|---|
| Cₘₐₓ (pmol/μl) | 33* | 5.23 | 8.57 | 0.59 |
| CL/F (nmol/(pmol/μl)/h) | 1.73 | 1.28 | 0.144 | 0.53 |
| AUC_{0-inf} (pmol/μl*h²) | 19.08 | 25.59 | 171 | 46 |
| R² | 0.99 | 0.99 | 0.99 | 0.94 |

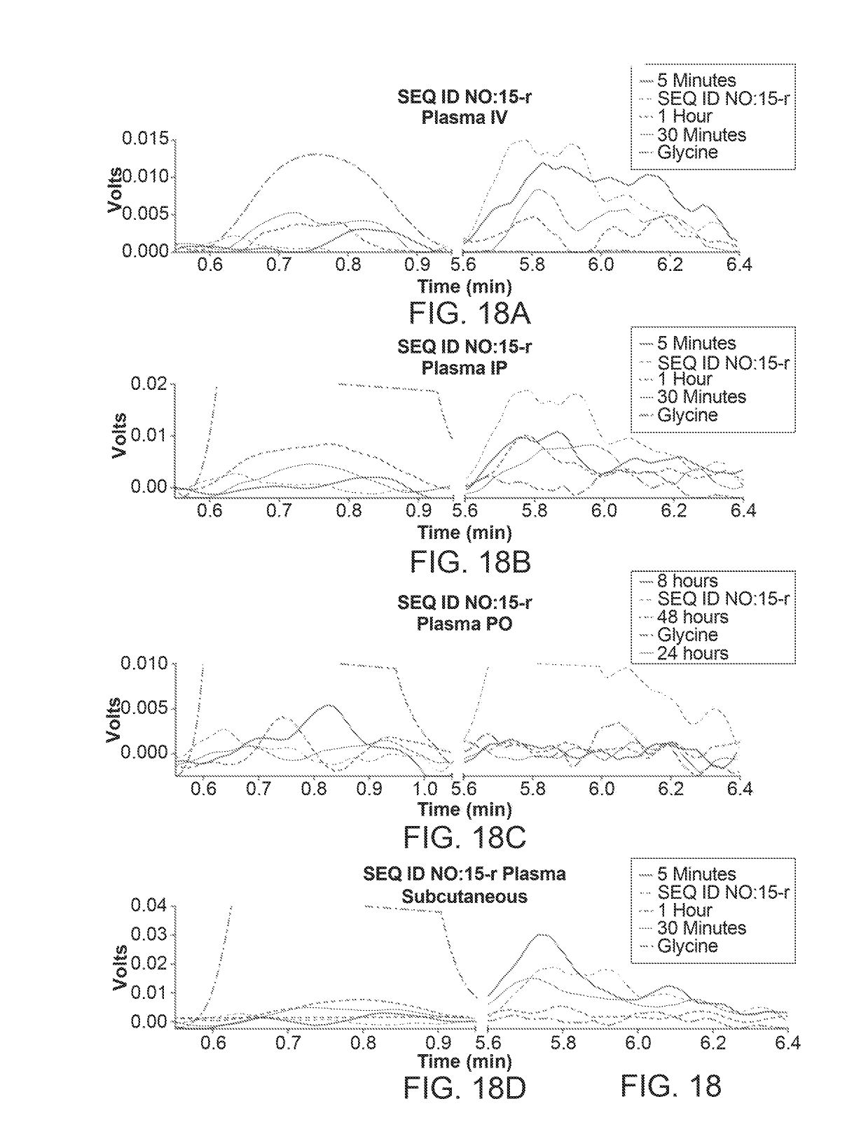

FIG. 18 shows analysis and quantification of signal in plasma by tandem HPLC and liquid scintillation of SEQ ID NO: 15-r at several time points after administration of 2 μCi/33 nmol of SEQ ID NO: 15-r in female Harlan athymic nude mice via different routes. HPLC was used to separate peptide fragments and liquid scintillation counting was used to quantify the radioactive signal of intact peptides or peptide fragment. SEQ ID NO: 15-r was spiked in as a positive control for intact peptide and Glycine was spiked in as a negative control for metabolized peptide. The peak at 0.5-1 min in each figure in FIG. 18 is glycine or breakdown products of the peptide. The peak at 6 min was intact peptide. FIG. 18A shows the signal in plasma after intravenous (IV) administration of SEQ ID NO: 15-r. SEQ ID NO: 15-r was spiked in as a positive control for intact peptide and Glycine was spiked in as a negative control for metabolized peptide. Following intravenous injection, intact peptide in plasma was decreased from 5 minutes to 1 hour and the signal baseline was reached by 3 hours. Breakdown products of the peptide did not increased over time. FIG. 18B shows the signal in plasma after intraperitoneal (IP) administration of SEQ ID NO: 15-r. SEQ ID NO: 15-r was spiked in as a positive control for intact peptide and Glycine was spiked in as a negative control for metabolized peptide. Following intraperitoneal injection, intact peptide in plasma was decreased from 5 minutes to 1 hour and the signal base line was reached by 3 hours. Breakdown products of the peptide were observed at the 1 hour time point. FIG. 18C shows the signal in plasma after oral (PO) administration of SEQ ID NO: 15-r. SEQ ID NO: 15-r was spiked in as a positive control for intact peptide and Glycine was spiked in as a negative control for metabolized peptide. Following oral administration, neither intact peptide or breakdown products of the peptide were observed in plasma. FIG. 18D shows the signal in plasma after subcutaneous (SC) administration of SEQ ID NO: 15-r. Following subcutaneous injection, intact peptide in plasma was decreased from 5 minutes to 1 hour. Some breakdown products of the peptide were observed at 30 minutes and at 1 hour.

TABLE 3 shows the percentage of the area under the curve (AUC) of intact peptide of SEQ ID NO: 15-r of the total radioactive signal (total AUC) in plasma. NP indicates that no radioactive peak was measurable and “0” indicates that no intact peptide was measured.

TABLE 3
AUC of SEQ ID NO: 15-r in Plasma

| | | IV | IP | PO | SC |
|---|---|---|---|---|---|
| 5 | min | 92 | 94 | | 100 |
| 30 | min | 100 | 100 | | 88 |
| 1 | hr | NP | 66 | | 50 |
| 3 | hr | NP | 0 | | |
| 8 | hr | | | 45 | |
| 24 | hr | | | NP | |
| 48 | hr | | | NP | |

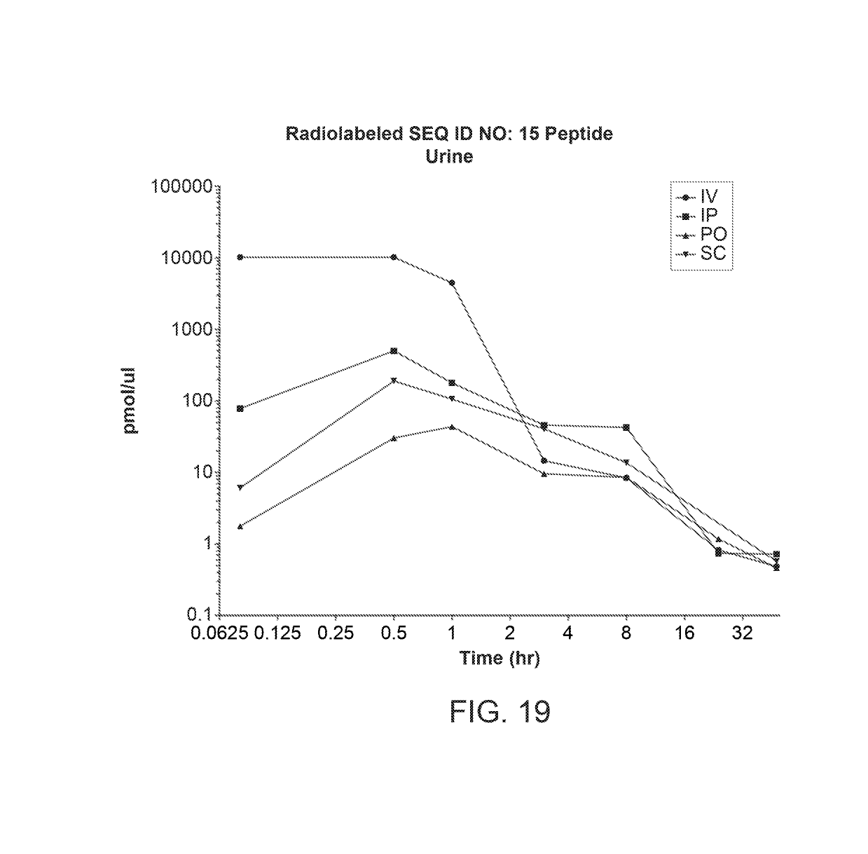

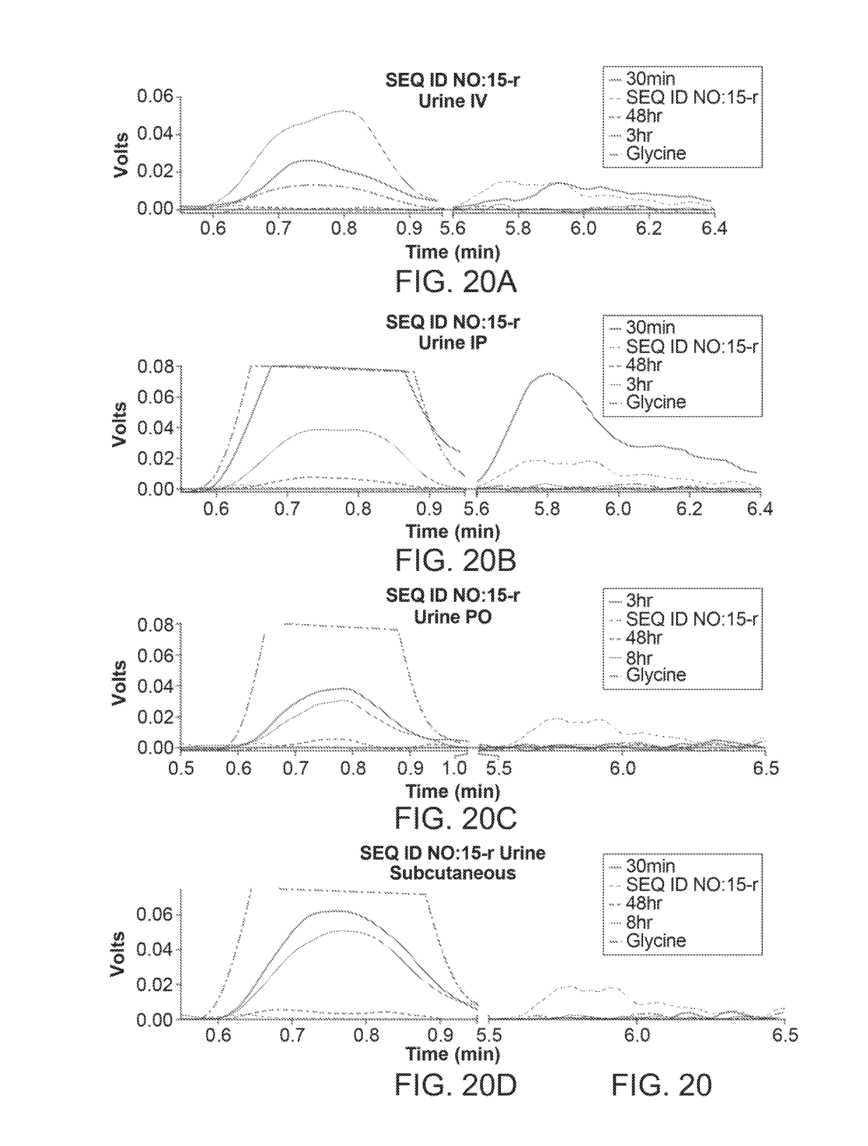

FIG. 19 shows liquid scintillation counting and quantification of the concentration of SEQ ID NO: 15-r in urine at several time points after administration of 2 μCi/33 nmol of SEQ ID NO: 15-r in female Harlan athymic nude mice via intravenous (IV) administration, intraperitoneal (IP) administration, oral (PO) administration, and subcutaneous (SC) administration. Each data point shows mean and error bars indicating standard deviation (n=3). SEQ ID NO: 15-r was spiked in as a positive control for intact peptide and Glycine was spiked in as a negative control for metabolized peptide. The peak at 0.5-1 min in each figure in FIG. 20 is glycine or breakdown products of the peptide. The peak at 6 min was intact peptide. FIG. 20 shows analysis and quantification of signal in urine by tandem HPLC and liquid scintillation of a SEQ ID NO: 15-r at several time points after administration of 2 μCi/33 nmol of SEQ ID NO: 15-r in female Harlan athymic nude mice via different routes. HPLC was used to separate peptide fragments and liquid scintillation counting was used to quantify the radioactive signal of intact peptides or peptide fragment. FIG. 20A shows the signal in urine after intravenous (IV) administration of a radiolabeled SEQ ID NO: 15-r. SEQ ID NO: 15-r was spiked in as a positive control for intact peptide and Glycine was spiked in as a negative control for metabolized peptide. Following intravenous injection, intact peptide in urine was decreased from 5 minutes to 1 hour and the signal baseline was reached by 3 hours. Breakdown products of the peptide were observed to increase from 5 minutes to 3 hours. FIG. 20B shows the signal in urine after intraperitoneal (IP) administration of SEQ ID NO: 15-r. SEQ ID NO: 15-r was spiked in as a positive control for intact peptide and Glycine was spiked in as a negative control for metabolized peptide. Following intraperitoneal injection, intact peptide in urine was increased from 5 minutes to 30 minutes and then dropped to baseline by 3 hours. Breakdown products of the peptide were observed at 30 minutes. FIG. 20C shows the signal in urine after oral (PO) administration of SEQ ID NO: 15-r. SEQ ID NO: 15-r was spiked in as a positive control for intact peptide and Glycine was spiked in as a negative control for metabolized peptide. Following oral administration, intact peptide was not observed in urine. Breakdown products of the peptide were observed in urine at 3 hours and 8 hours. FIG. 20D shows the signal in urine after subcutaneous (SC) administration of SEQ ID NO: 15-r. Following subcutaneous administration, intact peptide was not observed in urine. Breakdown products of the peptide were observed from 5 minutes to 8 hours.

TABLE 4 shows the percentage of the area under the curve (AUC) of intact peptide of SEQ ID NO: 15-r of the total radioactive signal (total AUC) in urine. “0” indicates that no intact peptide was measured.

TABLE 4
AUC of SEQ ID NO: 15-r in Urine

| | | IV | IP | PO | SC |
|---|---|---|---|---|---|
| 5 | min | 77 | 60 | | 0 |
| 30 | min | 47 | 49 | | 0 |
| 1 | hr | 43 | | | 0 |
| 3 | hr | 0 | 0 | 0 | |
| 8 | hr | 0 | | 0 | 0 |
| 24 | hr | 0 | 0 | | |
| 48 | hr | | 0 | 0 | 0 |

### Example 21

### Sensitivity and Specificity of Tumor Homing Peptides

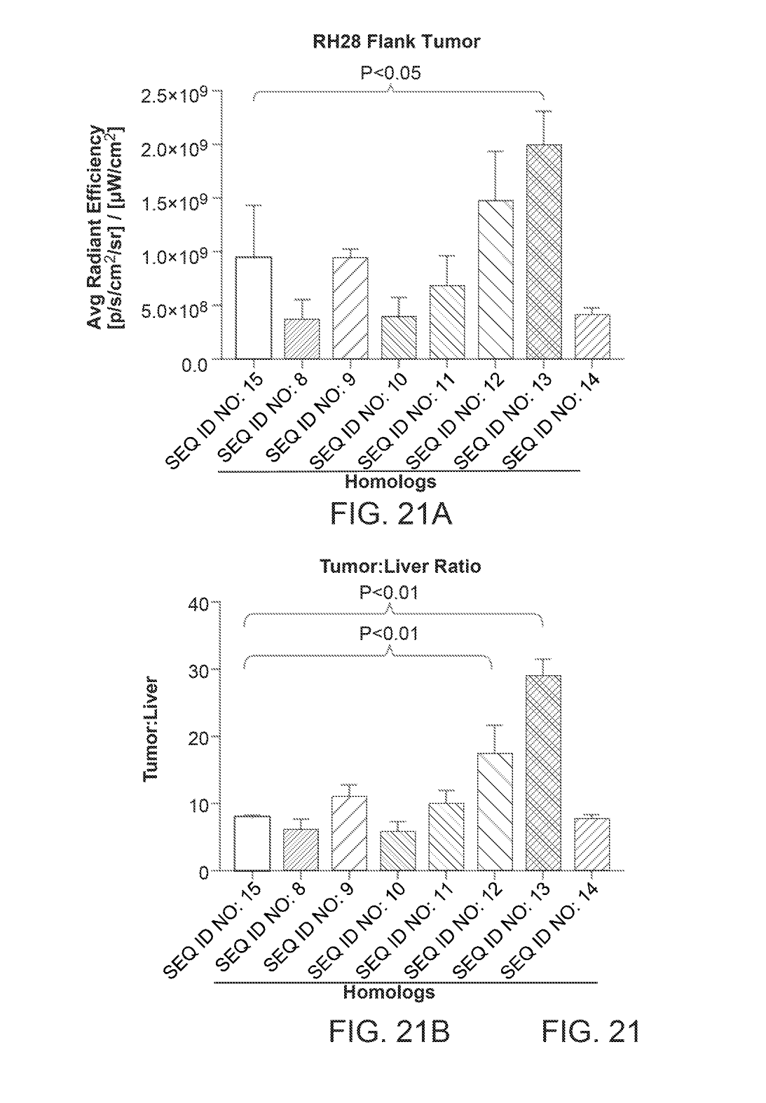

This example illustrates the sensitivity and specificity of tumor homing peptides in a mouse model and how different sequence variations effect tumor homing and peptide accumulation in a tumor. Tumor homing of various peptides of this disclosure was assessed by conjugating peptides to AlexaFluor 647 (AF647) and administering them in mice. Peptide of SEQ ID NO: 15, SEQ ID NO: 8, SEQ ID NO: 9, SEQ ID NO: 10, SEQ ID NO: 11, SEQ ID NO: 12, SEQ ID NO: 13, and SEQ ID NO: 14 were conjugated to AF647 and tested. FIG. 21 shows quantification of signal in tissues after administration of a panel of peptides conjugated to AF647 to mice. Peptide-AF647 conjugates were administered intravenously to mice bearing RH28 flank tumors at 10 nmol per mouse. Mice were euthanized four hours post-administration, organs were necropsied, and tissues were analyzed ex vivo using an IVIS imager. All tested sequences are CTI homologs. P-values were determined using an unpaired Student's t-test. FIG. 21A shows average radiant efficiency fluorescence from peptide-AF647 conjugates in RH28 flank tumors. FIG. 21B shows the tumor to liver ratio of average radiant efficiency fluorescence from peptide-AF647 conjugates. A peptide of SEQ ID NO: 13-AF647 conjugate exhibited the highest fluorescence in tumor tissues and peptides of SEQ ID NO: 12-AF647 conjugates and SEQ ID NO: 13-AF647 conjugates exhibited the highest fluorescence in tumor tissues after normalization to signal in liver. Overall, peptides of this disclosure displayed up to −30-fold higher signal in tumor (sensitivity) as compared to the liver, an off-target organ (specificity) and suggests the importance of peptide sequence for tumor binding. This shows the effect of different sequence variants in increasing (or decreasing) tumor accumulation and selectivity, which indicates preferred sequences or mutations.

### Example 22

### Chirality in Tumor Homing Peptides

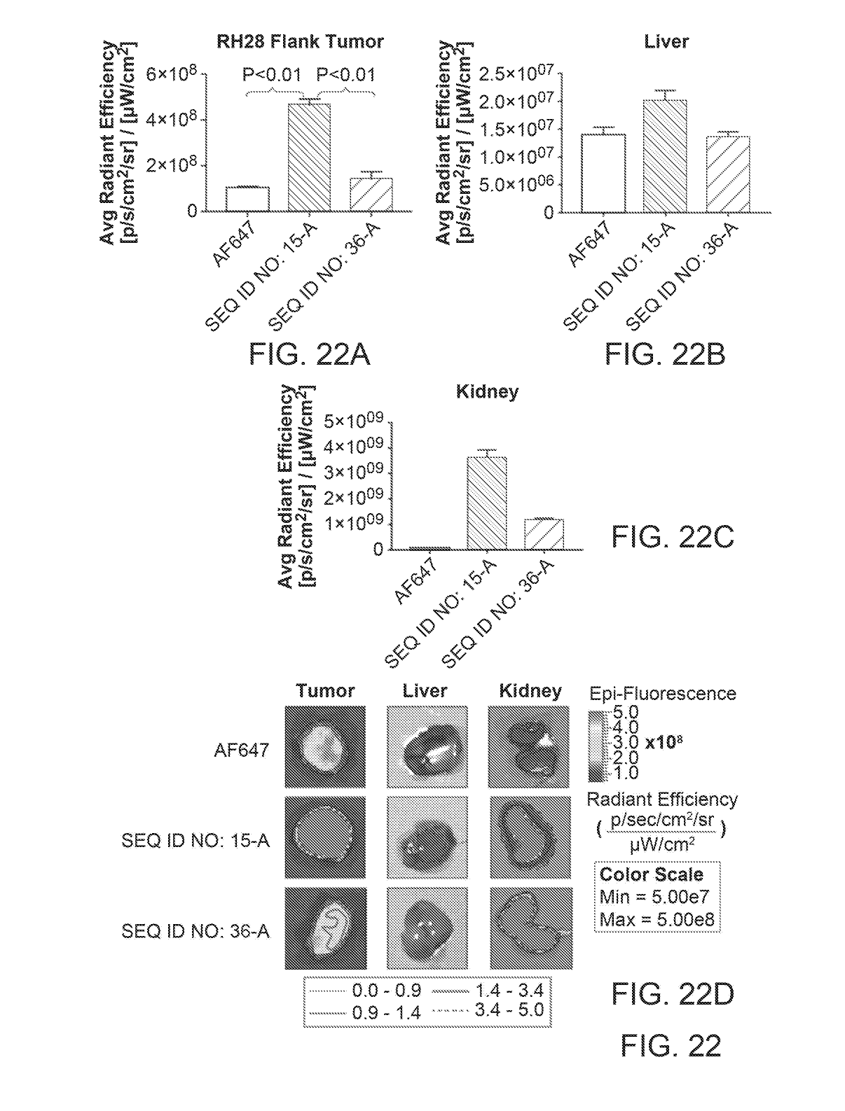

This example illustrates the role of chirality in tumor homing peptides of this disclosure. Each L-enantiomer in a peptide of SEQ ID NO: 15 was replaced with the corresponding D-enantiomer (SEQ ID NO: 36). Both peptides were conjugated to AlexaFluor 647 and tumor homing, and homing to the liver and kidneys was assessed by fluorescence quantification. FIG. 22 shows quantification of signal in tissues after administration of a peptide of SEQ ID NO: 15 conjugated to AF647 (SEQ ID NO: 15-A) and a peptide of SEQ ID NO: 36 (the D-amino acid version of SEQ ID NO: 15) conjugated to AF647 (SEQ ID NO: 36-A), and free AlexaFluor647 (AF647) to mice. Peptide-AF647 conjugates were administered intravenously to mice bearing RH28 flank tumors at 10 nmol per mouse. Mice were euthanized one hour post-administration, organs were necropsied, and tissues were analyzed ex vivo using an IVIS imager. P-values were determined using an unpaired Student's t-test. FIG. 22A shows average radiant efficiency fluorescence from SEQ ID NO: 15-A, SEQ ID NO: 36-A, and AF647 (as a negative control) in RH28 flank tumors. FIG. 22B shows average radiant efficiency fluorescence from SEQ ID NO: 15-A, SEQ ID NO: 36-A, and AF647 (as a negative control) in livers. FIG. 22C shows average radiant efficiency fluorescence from SEQ ID NO: 15-A, SEQ ID NO: 36-A, and AF647 (as a negative control) in kidneys. FIG. 22D shows fluorescence images of radiant efficiency in necropsied tumor, liver, and kidney taken using an IVIS imager, which correspond to the bar graphs in FIG. 22A, FIG. 22B, and FIG. 22C. A representative tissue is shown from one mouse in each group—AF647, SEQ ID NO: 15-A, and SEQ ID NO: 36-A. As shown in FIG. 22A, a peptide of SEQ ID NO: 15 exhibited higher homing to flank tumor than a peptide of SEQ ID NO: 36. These results show that L-amino acids are important for the tumor homing properties of peptides of this disclosure and suggest the importance of the 3D peptides structure, which may be important for binding to a chiral protein receptor displayed on tumor cells as a mechanism for uptake and accumulation of peptides of this disclosure in a tumor.

### Example 23

### Competitive Homing Assay

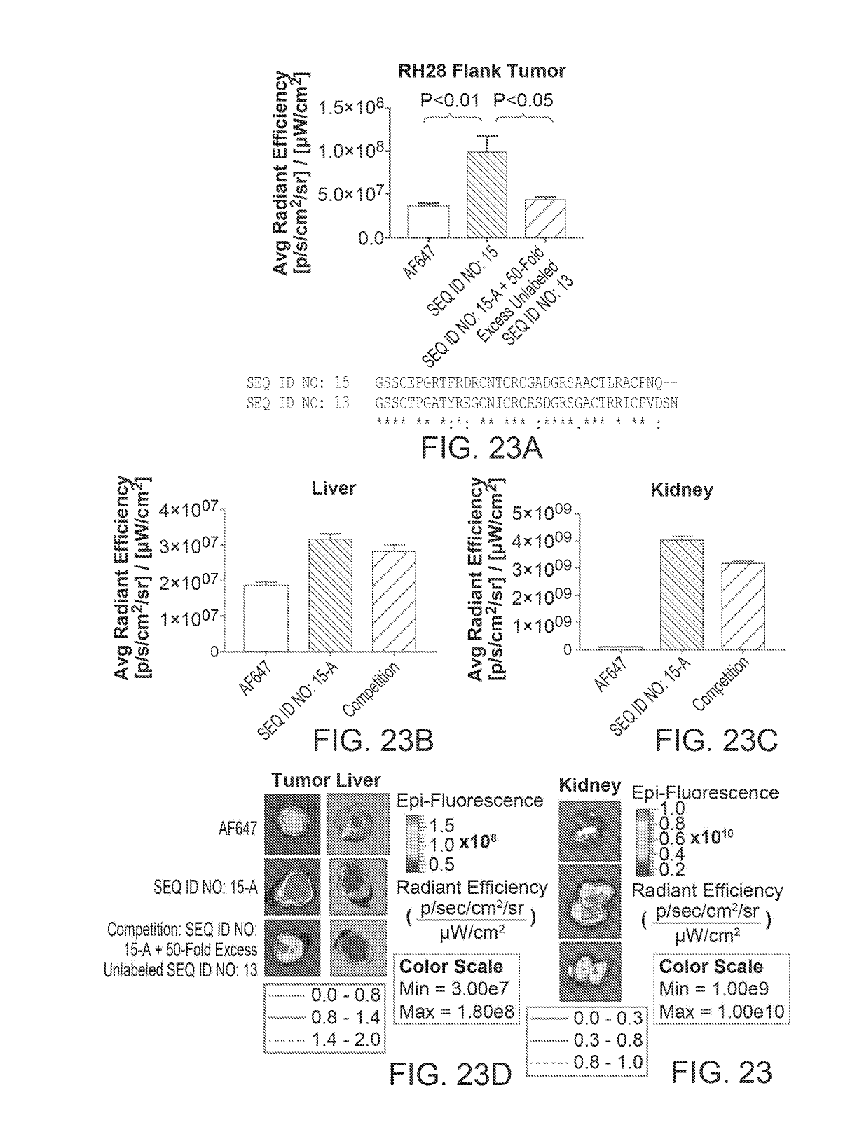

This example illustrates a competitive homing assay to assess the strength and specificity of tumor homing of peptides of this disclosure. FIG. 23 shows quantification of signal in tissues one hour after intravenous administration of a peptide of SEQ ID NO: 15 conjugated to AF647 (SEQ ID NO: 15-A) (2 nmol per mouse), simultaneous intravenous administration of a peptide of SEQ ID NO: 15-A (2 nmol per mouse) with a 50-fold excess of unlabeled SEQ ID NO: 13 (98 nmol per mouse), or intravenous administration of free fluorophore (AF647) (2 nmol). P-values were determined using an unpaired Student's t-test. FIG. 23A shows average radiant efficiency fluorescence from SEQ ID NO: 15-A, SEQ ID NO: 15-A with a 50-fold excess of unlabeled SEQ ID NO: 13, and AF647 (as a negative control) in RH28 flank tumors. FIG. 23B shows average radiant efficiency fluorescence from SEQ ID NO: 15-A, SEQ ID NO: 15-A with a 50-fold excess of unlabeled SEQ ID NO: 13, and AF647 (as a negative control) in livers. FIG. 23C shows average radiant efficiency fluorescence from SEQ ID NO: 15-A, SEQ ID NO: 15-A with a 50-fold excess of unlabeled SEQ ID NO: 13, and AF647 (as a negative control) in kidneys. FIG. 23D shows fluorescence images of radiant efficiency in necropsied tumor, liver, and kidney taken using an IVIS imager, which correspond to the bar graphs in FIG. 23A, FIG. 23B, and FIG. 23C. A representative tissue is shown from one mouse in each group—AF647, SEQ ID NO: 15-A, and SEQ ID NO: 15-A with a 50-fold excess of unlabeled SEQ ID NO: 13. SEQ ID NO: 15-A significantly outcompeted a 50-fold excess of SEQ ID NO: 13, as demonstrated by the high fluorescence signal observed in tumors. These results suggest that binding to tumors is saturable and that may be due to an area of shared sequence or pharmacophore between SEQ ID NO: 13 and SEQ ID NO: 15. The results suggest that SEQ ID NO: 13 and SEQ ID NO: 15 bind to the same receptor and indicate a specific receptor may be important for uptake and accumulation of peptides of this disclosure in a tumor.

### Example 24

### Tumor Cell Viability

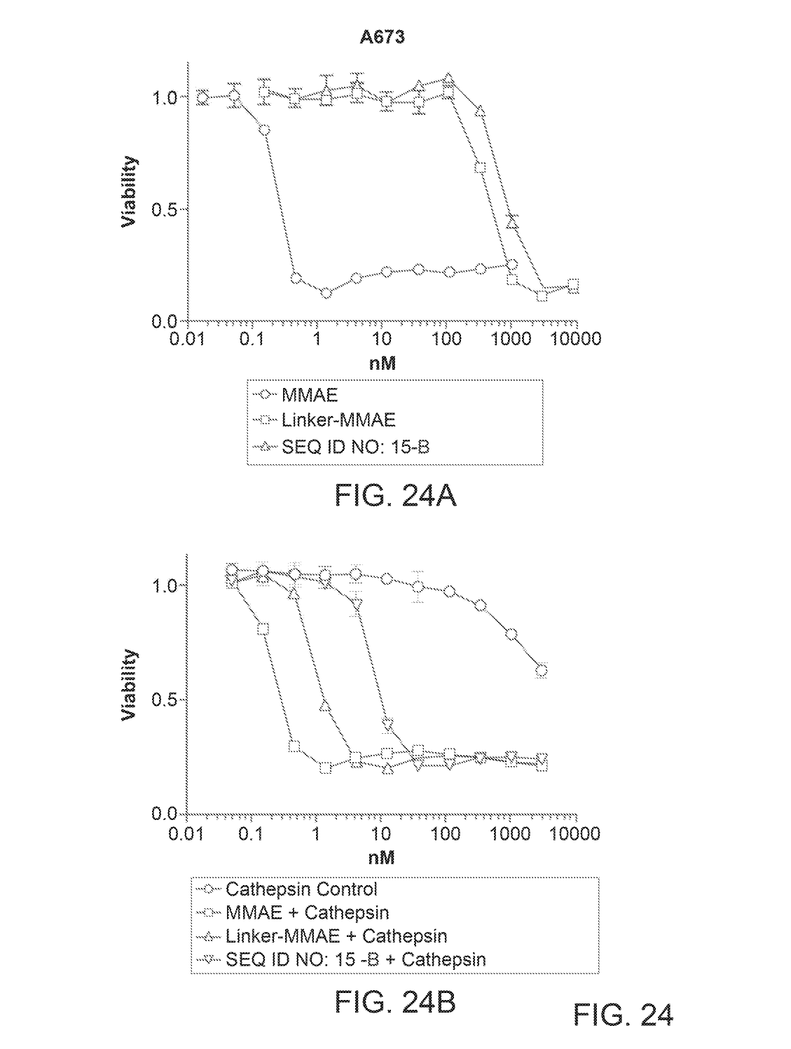

This example illustrates tumor cell viability to peptide-drug conjugates of this disclosure. A peptide of SEQ ID NO: 15 was conjugated to MMAE and evaluated for the ability to induce death in A673 tumor cells. To evaluate the role of the linker used to conjugate peptides of this disclosure to drugs, tumor cell viability was also evaluated after incubation with cathepsins, an enzyme that cleaves the linker. FIG. 24 shows cell viability curves after treatment with monomethyl auristatin E (MMAE), MMAE with a Val-Cit-PAB linker (linker-MMAE), and MMAE conjugated to a peptide of SEQ ID NO: 15 via a Val-Cit-PAB linker (SEQ ID NO: 15-B). A673 Ewing's sarcoma cells were treated for two days without or with cathepsins, an enzyme that cleaves the linker, and with increasing concentrations of MMAE, linker-MMAE, or SEQ ID NO: 15-B. Cell viability was assessed using a Cell Titer Glo assay. FIG. 24A shows cell viability curves for A673 cells incubated with MMAE, linker-MMAE, or SEQ ID NO: 15-B. FIG. 24B shows cell viability curves for A673 cells incubated with cathepsins, MMAE and cathepsin, linker-MMAE and cathepsin, or SEQ ID NO: 15-B and cathepsin.

These results suggest that the Val-Cit-PAB linker may allow for decreased toxicity in off-target tissues. These results also show that Val-Cit-PAB linkers which can be cleaved by naturally occurring enzymes are useful in restoring the potency of peptide-drug conjugates of this disclosure. Other linkers are used in peptide-drug conjugates of the disclosure, which are cleaved more effectively by cancer cells, thereby enhancing the potency of the peptide-drug conjugate. Choosing a linker that is cleaved by enzymes that are present or overexpressed in the tumor environment can allow for increased delivery of an active agent by the peptide and/or increase the therapeutic window of delivery of the active agent.

### Example 25

### Small Molecule Drug Enhancing Properties of Peptides

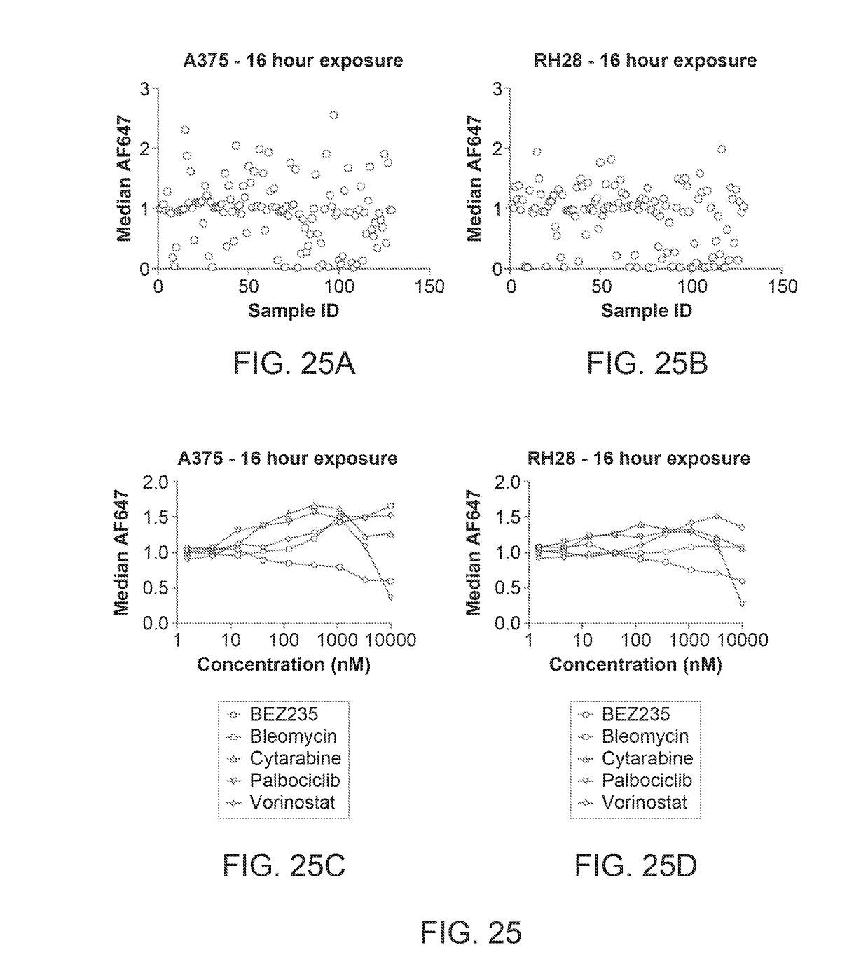

This example illustrates the small molecule drug enhancing properties of peptides of this disclosure. Peptides of this disclosure were useful in enhancing or dampening the potency of several small molecule drugs. FIG. 25 shows that small molecules can modulate the efficacy of a tumor targeting peptide-dye conjugate of this disclosure. The Approved Oncology Drugs Plated Set VII was obtained from NCI and included histone deacetylase (HDAC) inhibitors such as vorinostat, belinostat, panobinostat, and pentostatin. The tested set also included tyrosine kinase inhibitors, protease inhibitors, and anthracyclines. A375 cells and RH28 cells were plated at 10,000 cells per well in 96 well plates and allowed to adhere overnight. FIG. 25A shows the median fluorescence for each drug tested in A375 cells. Drugs from the 129 compound set were added at 10 μM and allowed to incubate for 16 hours. A peptide of SEQ ID NO: 15 conjugated to AlexaFluor647 (SEQ ID NO: 15-A) was added at 1 μM for four hours. Cells were washed three times with PBS-FBS and once in PBS. Cells were trypsinized, resuspended in PBS-FBS-DAPI, and assessed for average fluorescence as compared to untreated cells by flow cytometry analysis. Each data point represents a drug from Approved Oncology Drugs Plated Set VII incubated with SEQ ID NO: 15-A. Median fluorescence above 1 indicates that the efficacy was enhanced and median fluorescence below 1 indicates that the efficacy was reduced. FIG. 25B shows the median fluorescence for each drug tested in RH28 cells. Drugs from the 129 compound set were added at 10 μM and allowed to incubate for 16 hours. A peptide of SEQ ID NO: 15 conjugated to AlexaFluor647 (SEQ ID NO: 15-A) was added at 1 μM for four hours. Cells were washed three times with PBS-FBS and once in PBS. Cells were trypsinized, resuspended in PBS-FBS-DAPI, and assessed for average fluorescence as compared to untreated cells by flow cytometry analysis. Each data point represents a drug from the Approved Oncology Drugs Plated Set VII incubated with SEQ ID NO: 15-A. Median fluorescence above 1 indicates that the efficacy was enhanced and median fluorescence below 1 indicates that the efficacy was reduced. FIG. 25C shows the median fluorescence in A375 cells for five drugs from the Approved Oncology Drugs Plated Set VII including BEZ235, bleomycin, cytarabine, palbociclib, and vorinostat. Each drug was administered at increasing concentrations with 1 μM of peptide of SEQ ID NO: 15 conjugated to AlexaFluor647 (SEQ ID NO: 15-A) and the median fluorescence was plotted. Median fluorescence above 1 indicates that the efficacy was enhanced and median fluorescence below 1 indicates that the efficacy was reduced.

FIG. 25D shows the median fluorescence in RH28 cells for five drugs from the Approved Oncology Drugs Plated Set VII including BEZ235, bleomycin, cytarabine, palbociclib, and vorinostat. Each drug was administered at increasing concentrations with 1 μM of peptide of SEQ ID NO: 15 conjugated to AlexaFluor647 (SEQ ID NO: 15-A) and the median fluorescence was plotted. Median fluorescence above 1 indicates that the efficacy was enhanced and median fluorescence below 1 indicates that the efficacy was reduced.

These results showed that small molecule drugs, including BEZ235, bleomycin, cytarabine, palbociclib, and vorinostat, can modulate the accumulation of peptides of this disclosure to tumor cells. Moreover, enhancement or reduction of accumulation of peptides of this disclosure by small molecule drugs was a concentration-dependent phenomenon.

### Example 26

### Solved Crystal Structures of Peptide Homologs

This example illustrates the solved crystal structures of peptide homologs including peptides of SEQ ID NO: 10-SEQ ID NO: 12, SEQ ID NO: 14 and SEQ ID NO: 26. For each peptide, the peptide was resuspended at a target concentration of 80 mg/mL. Crystallization screening was performed at room temperature by vapor diffusion, with 1:1 protein solution:reservoir solution sitting drops, set up using the Nextal JCSG+, PEGs, and (NH₄)₂SO₄ factorial suites (Qiagen) and sub-microliter robotics (TTP Labtech mosquito). Diffraction data were collected from single crystals using a Rigaku MicroMax-007 HF home source or beamline 5.0.1 at the Advanced Light Source (Lawrence Berkley National Laboratory, Berkeley, Calif.). Initial phases were determined either by molecular replacement (MR), using PHASER (McCoy, A. J., J Appl Crystallogr., 40 (Pt 4): 658-674 (2007)) in the CCP4 program suite (Winn, M. D., Acta Crystallogr D Biol Crystallogr., 67 (Pt 4): 235-42 (2011)) using homologous structures from the RCSB PDB (Berman, H. M., Nucleic Acids Res., 28(1): 235-42 (2000)) as search models, or sulfur single-wavelength anomalous diffraction (sSAD) (Liu, Q., Acta Crystallogr D Biol Crystallogr., 69 (Pt 7): 1314-32 (2013)), using CuKalpha radiation to maximize the anomalous signal, and determining sulfur substructures with SHELX (Sheldrick, G. M., Acta Crystallogr D Biol Crystallogr., 66 (Pt 4): 479-85 (2010)). For sSAD phasing, Bijvoet pair measurement was optimized by collecting data through 5° wedges with alternating phi rotations of 180°, in 1° oscillations. Data were reduced and scaled with HKL2000 (Otwinowski, Z., Methods Enzymol., 276: 307-326 (1997)). Iterative cycles of model building and refinement were performed with COOT (Emsely, P., Acta Crystallogr D Biol Crystallogr., 60 (Pt 12 Pt 1): 2126-32 (2004)) and REFMAC (Murshudov, G. N., Acta Crystallogr D Biol Crystallogr., 53 (Pt 3): 240-55 (1997)). Structure validation was performed with MolProbity (Davis, I. W., Nucleic Acids Res., 35 (Web Server issue): W375-83 (2007)).

Similarly, the crystal structure of any one of peptides of SEQ ID NO: 1-SEQ ID NO: 9, SEQ ID NO: 13, SEQ ID NO: 15-SEQ ID NO: 25, or SEQ ID NO: 27-SEQ ID NO: 72 are solved using the above methods.

FIG. 26 shows structural analysis of SEQ ID NO: 10 peptide, SEQ ID NO: 11 peptide, SEQ ID NO: 12 peptide, SEQ ID NO: 14 peptide, and SEQ ID NO: 26 peptide based on the crystal structures determined as described above. FIG. 26A shows a cartoon representation of structural alignment of SEQ ID NO: 10 peptide, SEQ ID NO: 11 peptide, SEQ ID NO: 12 peptide, SEQ ID NO: 14 peptide, and SEQ ID NO: 26 peptide with a pacifastin structural fold with a 180° view along the X axis. Ovals indicate molecular surface involved in chymotrypsin binding and inhibition. FIG. 26B shows a surface representation of SEQ ID NO: 10 peptide, SEQ ID NO: 11 peptide, SEQ ID NO: 12 peptide, SEQ ID NO: 14 peptide, SEQ ID NO: 26 peptide with chymotrypsin binding site represented by medium-colored gray and dark gray shades with medium-colored gray shades indicating conserved sequences. FIG. 26C shows a general sequence motif and logo for a peptide that can bind chymotrypsin with chymotrypsin binding sites indicated by arrows (speckled and unfilled) and conserved residues indicated by unfilled arrows (N=8; SEQ ID NO: 10-SEQ ID NO: 12, SEQ ID NO: 14, and SEQ ID NO: 81-SEQ ID NO: 83). FIG. 26D shows a sequence motif for a peptide that exhibits tumor homing propensity (N=35; SEQ ID NO: 1-SEQ ID NO: 35). FIG. 26E shows a cartoon representation of SEQ ID NO: 10 peptide, SEQ ID NO: 11 peptide, SEQ ID NO: 12 peptide, SEQ ID NO: 14 peptide, and SEQ ID NO: 26 peptide. FIG. 26F shows an electrostatic surface representation of the cartoon representations of SEQ ID NO: 10 peptide, SEQ ID NO: 11 peptide, SEQ ID NO: 12 peptide, SEQ ID NO: 14 peptide, and SEQ ID NO: 26 peptide as shown in FIG. 26E at 180° along the Y axis. FIG. 26 shows structural analysis of SEQ ID NO: 10 peptide, SEQ ID NO: 11 peptide, SEQ ID NO: 12 peptide, SEQ ID NO: 14 peptide, and SEQ ID NO: 26 peptide.

### Example 27

### Paclitaxel Peptide Conjugates

This example illustrates the synthesis of paclitaxel peptide conjugates. A paclitaxel peptide conjugate was made by conjugating paclitaxel to a peptide of SEQ ID NO: 15 (SEQ ID NO: 15-P). SEQ ID NO: 15-P were made by activating the paclitaxel by using the general dicarboxylic acid method, the general cyclic anhydride method, or with an N-hydroxysuccinimide ester.

General Dicarboxylic Acid Method.

1-Ethyl-3-(3-dimethylaminopropyl)carbodiimide hydrochloride (EDC; 0.64 mmol), 4-dimethylaminopyridine (DMAP; 0.64 mmol), and dicarboxylic acid (0.64 mmol), such as trans-1,4-cyclohexanedicarboxylic acid, were dissolved in anhydrous acetone (5 mL) and were added dropwise to a stirred solution of Paclitaxel (0.59 mmol) in anhydrous acetone (5 mL). The reaction was stirred at room temperature for 2 hours. After this time, the organic solvent was removed under vacuum, and the residue was partitioned between ethyl acetate and 0.1 M hydrochloric acid. The organic layer was further washed with brine, and the organic layer was dried over anhydrous sodium sulfate. The organic solution was filtered and was removed under vacuum. The crude product was dissolved in minimal dimethylsulfoxide (DMSO), and purified by preparative high-performance liquid chromatography (HPLC).

General Cyclic Anhydride Method.

Paclitaxel (0.13 mmol), DMAP (0.15 mmol), and cyclic anhydride (0.15 mmol), such as glutaric anhydride, were dissolved in anhydrous acetone (2 mL). The reaction was stirred overnight at room temperature. After this time the acetone was removed under reduced pressure. The product was partitioned between ethyl acetate and 0.1 M hydrochloric acid. The organic layer was further washed with brine, and the organic layer was dried over anhydrous sodium sulfate. The organic solution was filtered and removed under vacuum to afford the title compound as a white solid. The product was used without further purification.

N-Hydroxysuccinimide Ester of Paclitaxel-Linker-Carboxylic Acid.

Paclitaxel-linker-carboxylic acid (0.45 mmol), EDC (0.55 mmol) and N-hydroxysuccinimide (NHS-OH; 0.55 mmol) were weighed into a reaction vessel and dissolved in anhydrous DMSO (2 mL). The reaction progress was monitored by liquid chromatography-mass spectrometry (LC-MS). Additional EDC and NHS-OH were added if there was any free Paclitaxel-linker-carboxylic acid remaining. Once determined to be complete, the crude reaction mixture was used as is, or the title compound was purified using preparative HPLC.

Conjugation of Paclitaxel to Peptides.

A peptide of SEQ ID NO: 15 (1.2 μmol) was dissolved to 2.5 mg/mL in anhydrous DMSO along with N-methylmorpholine (NMM; 13 μmol) and stirred at room temperature. The paclitaxel-linker-NHS ester (1.3 μmol) was dissolved at 10 mg/mL in anhydrous DMSO and was added in 10 portions over 30 minutes. After the final addition, the reaction was stirred for 30 minutes. The reaction was then monitored by LC-MS, and if unmodified SEQ ID NO: 15 peptide remains, additional NHS ester were added until no more SEQ ID NO: 15 peptide remains. The SEQ ID NO: 15-P product was purified by preparative HPLC. A small sample of each fraction was set aside for LC-MS analysis. The fractions were individually frozen in a dry ice/acetone bath, and lyophilized.

Additional peptide-paclitaxel conjugates are synthesized by conjugating any peptide of this disclosure (e.g., SEQ ID NO: 1-SEQ ID NO: 14, SEQ ID NO: 16-SEQ ID NO: 35, or SEQ ID NO: 37 -SEQ ID NO: 71) to paclitaxel. These paclitaxel peptide conjugates are made by activating the paclitaxel by using the general dicarboxylic acid method, the general cyclic anhydride method, or with an N-hydroxysuccinimide ester and then are conjugated as described above.

### Example 28

### Treatment of Cancer

This example illustrates treatment of cancer with any peptide of this disclosure (e.g., SEQ ID NO: 1-SEQ ID NO: 35 or SEQ ID NO: 37-SEQ ID NO: 71). A peptide of the present disclosure is recombinantly expressed or chemically synthesized and is used directly, after radiolabeling, or after conjugation to a fluorophore or therapeutic compound, such as monomethyl auristatin E, paclitaxel, DM1 (mertansine), pyrrolobenzodiazepine dimer, or calicheamicin. The peptide or peptide conjugate is administered in a pharmaceutical composition to a subject as a therapeutic for cancer. One or more peptides or peptide conjugates of the present disclosure are administered to a subject. A subject can be a human or an animal. The pharmaceutical composition is administered subcutaneously, intravenously, orally, intramuscularly, mucosally, or intraperitoneally. The peptides or peptide conjugates target tumor cells affected by cancer.

While preferred embodiments of the present invention have been shown and described herein, it will be apparent to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-146. (canceled)

147. A peptide comprising:
a sequence that has at least 89% sequence identity with SEQ ID NO: 51 or a functional fragment thereof; or
a sequence that has at least 89% sequence identity with any one of SEQ ID NO: 48, SEQ ID NO: 49, SEQ ID NO: 45, SEQ ID NO: 47, SEQ ID NO: 1, SEQ ID NO: 15, SEQ ID NO: 12, SEQ ID NO: 13, SEQ ID NO: 9, SEQ ID NO: 11, or a functional fragment thereof.

148. The peptide of claim 147, wherein the peptide comprises a knotted peptide.

149. The peptide of claim 147, wherein the peptide comprises at least 6 cysteine residues.

150. The peptide of claim 147, wherein the peptide comprises a plurality of disulfide bridges formed between cysteine residues.

151. The peptide of claim 147, wherein the functional fragment comprises at least 29 residues and has at least 89% sequence identity with SEQ ID NO: 51.

152. The peptide of claim 147, wherein the peptide is conjugated to an active agent or a detectable agent.

153. The peptide of claim 152, wherein the active agent is selected from the group consisting of: a peptide, an oligopeptide, a polypeptide, a polynucleotide, a polyribonucleotide, a DNA, a cDNA, a ssDNA, a RNA, a dsRNA, a micro RNA, an oligonucleotide, an antibody, a single chain variable fragment (scFv), an antibody fragment, a cytokine, a hormone, a growth factor, a checkpoint inhibitor, an immune modulator, a neurotransmitter, a chemical agent, a cytotoxic molecule, a toxin, a radiosensitizer, a radioprotectant, a therapeutic small molecule, a nanoparticle, a liposome, a polymer, a dendrimer, an enzyme, a chemokine, a chemical agent, a fatty acid, a peptidomimetic, a complement fixing peptide or protein, polyethylene glycol, a lipid, an Fc region, a metal, a metal chelate, a steroid, a corticosteroid, an anti-inflammatory agent, an immunosuppressant, a protease inhibitor, an amino sugar, a chemotherapeutic, a cytotoxic chemical, a tyrosine kinase inhibitor, an anti-infective agent, an antibiotic, an anti-viral agent, an anti-fungal agent, an aminoglycoside, a nonsteroidal anti-inflammatory drug (NSAID), a statin, a biopolymer, a polysaccharide, a proteoglycan, an immunomodulatory agent, a T cell activating agent, a macrophage activating agent, a natural killer cell activating agent, and a glycosaminoglycan.

154. The peptide of claim 152, wherein the active agent inhibits a protease, has antimicrobial activity, has anticancer activity, has anti-inflammatory activity, or any combination thereof.

155. The peptide of claim 152, wherein the active agent is a chemotherapeutic agent.

156. The peptide of claim 153, wherein the cytotoxic molecule is an auristatin, a maytansinoid, MMAE, DM1, DM4, doxorubicin, a calicheamicin, a platinum compound, cisplastin, a taxane, paclitaxel, a BACE inhibitor, a Bcl-xL inhibitor, WEHI-539, venetoclax, ABT-199, navitoclax, AT-101, obatoclax, a pyrrolobenzodiazepine, pyrrolobenzodiazepine dimer, or dolastatin.

157. The peptide of claim 152, wherein the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator.

158. The peptide of claim 152, wherein the detectable agent is a fluorescent dye.

159. The peptide of claim 152, and wherein the peptide, active agent, detectable agent, or any combination thereof penetrates a solid tumor.

160. The peptide of claim 147, wherein the peptide homes, targets, migrates to, distributes to, accumulates in, or is directed to a tumor or cancerous cell.

161. The peptide of claim 160, wherein the tumor or cancerous cell is from a sarcoma, cervical cancer, B cell lymphoma, breast cancer, brain cancer, Ewing sarcoma, Burkitt's lymphoma, medulloblastoma, rhabdomyosarcoma, colorectal cancer, or melanoma.

162. A method of treating a condition in a subject in need thereof, the method comprising treating the condition by administering to the subject a peptide conjugate, the peptide conjugate comprising, a peptide and an active agent, the peptide comprising:
a sequence that has at least 89% sequence identity with SEQ ID NO: 51, or a functional fragment thereof; or
a sequence that has at least 89% sequence identity with any one of SEQ ID NO: 48, SEQ ID NO: 49, SEQ ID NO: 45, SEQ ID NO: 47, SEQ ID NO: 1, SEQ ID NO: 15, SEQ ID NO: 12, SEQ ID NO: 13, SEQ ID NO: 9, SEQ ID NO: 11, or a functional fragment thereof.

163. The method of claim 162, wherein the administering comprises administration by inhalation, intranasally, orally, topically, intravenously, subcutaneously, intraarticularly, intramuscularly, intrathecally, intraperitoneally, or any combination thereof.

164. The method of claim 162, wherein the condition is a tumor or cancer.

165. The method of claim 162, wherein the condition is a solid tumor.

166. The method of claim 162, wherein the condition is a sarcoma, cervical cancer, B cell lymphoma, breast cancer, brain cancer, Ewing sarcoma, Burkitt's lymphoma, medulloblastoma, rhabdomyosarcoma, colorectal cancer, or melanoma.

167. The method of claim 162, the method further comprising treating the subject with chemotherapy, radiation therapy, or immunomodulatory therapy.

168. The method of claim 162, wherein the peptide conjugate further comprises a detectable agent.

169. The method of claim 162, wherein the peptide conjugate comprises up to 10 active agents.

170. A method of imaging an organ or body region of a subject, the method comprising, imaging the organ or body region of the subject by administering to the subject a peptide conjugate, the peptide conjugate comprising, a peptide and a detectable agent, the peptide comprising:
a sequence that has at least 89% sequence identity with SEQ ID NO: 51, or a functional fragment thereof; or
a sequence that has at least 89% sequence identity with any one of SEQ ID NO: 48, SEQ ID NO: 49, SEQ ID NO: 45, SEQ ID NO: 47, SEQ ID NO: 1, SEQ ID NO: 15, SEQ ID NO: 12, SEQ ID NO: 13, SEQ ID NO: 9, SEQ ID NO: 11, or a functional fragment thereof, and imaging the organ or body region of the subject.

171. The method of claim 170, the method further comprising detecting a cancer or diseased region, tissue, structure or cell of the subject.

172. The method of claim 170, the method further comprising treating the cancer by performing surgery on the subject to remove the cancer or the diseased region, tissue, structure or cell of the subject.

173. The method of claim 172, the method further comprising imaging the cancer or diseased region, tissue, structure, or cell of the subject after the surgery.

174. The method of claim 170, the peptide conjugate further comprising an active agent.

175. The method of claim 170, the peptide conjugate comprising up to 10 detectable agents.
